(19) United States
(12) Patent Application Publication
(10) Pub. No.: US 2005/0095674 A1
(43) Pub. Date: May 5, 2005

(54) Cannabinoid receptor interacting proteins and methods of use

(76) Inventors: Deborah L. Lewis, Evans, GA (US); Sheela Bhartur, Nashville, TN (US); Kathleen Wallis, Aiken, SC (US); Jason Niehaus, Augusta, GA (US)

Correspondence Address: SUTHERLAND ASBILL & BRENNAN LLP, 999 PEACHTREE STREET, N.E., ATLANTA, GA, 30309, US

(21) Appl. No.: 10/898,406

(22) Filed: Jul. 23, 2004

Related U.S. Application Data
(60) Provisional application No. 60/489,542, filed on Jul. 23, 2003.
(60) Provisional application No. 60/548,697, filed on Feb. 27, 2004.

(51) Int. Cl. A61K 38/17; C07H 21/04; C07K 14/705

(52) U.S. Cl. 435/69.1; 514/12; 530/350; 435/320.1; 435/325; 536/23.5

(57) ABSTRACT

Novel polypeptides capable of interacting with the CB1 cannabinoid receptor. Also provided are genomic and cDNA sequences encoding the CB1 receptor interacting proteins 1a and 1b (CRIP1a and CRIP1b) and antibodies to the CRIP1a and CRIP1b proteins. Also provided are methods of modulating the activity of the CB1 receptor and methods of screening for modulators of CRIP1a and CRIP1b activity on the CB1 receptor.

### CROSS-REFERENCE TO RELATED APPLICATIONS

The present application claims the priority benefit of U.S. Provisional Patent Application Ser. No. 60/489,542 filed Jul. 23, 2003 and U.S. Provisional Patent Application Ser. No. 60/548,697 filed Feb. 27, 2004, the entire contents of both applications are hereby incorporated by reference.

### ACKNOWLEDGMENT OF FEDERAL RESEARCH SUPPORT

This invention was made, at least in part, with funding from the National Institutes of Health (NIH Grant Number DA10350 to DL). Accordingly, the United States Government has certain rights in this invention.

### BACKGROUND OF THE INVENTION

1. Field of the Invention

This invention relates generally to nucleic acid sequences encoding polypeptides that associate with the CB1 cannabinoid receptor. In particular, this invention relates to nucleic acid sequences encoding the CB1 cannabinoid receptor interacting proteins 1a and 1b (CRIP1a and CRIP1b), CRIP1a and CRIP1b polypeptides, and methods of their use to treat patients suffering from various conditions.

2. Background Art

Cannabinoids, including (−)-Δ9-THC, the active principle in marijuana, and anadamide, an endogenous agonist, have a multitude of effects including alterations in cognition and memory, analgesia, anticonvulsion, immunosuppression, and appetite stimulation (Felder et al., 1993, Proc. Natl. Acad. Sci. USA, 90:7656-7660). Cannabinoids are therapeutic in that they decrease intestinal motility in the case of diarrhea, decrease bronchial constriction in asthma, decrease intraocular pressure in glaucoma, contribute to the control of tremor in multiple sclerosis, and are antirheumatic and antipyretic.

Although cannabinoids are lipophilic, most of their biological actions are mediated via G-protein-coupled receptors in the plasma membrane (Howlett, 1995, Ann. Rev. Pharmacol. Toxicol., 35:607-634). Two receptor subtypes, CB1 and CB2, have been cloned, and their localization has been studied using mRNA analysis and immunochemistry of receptors (Matsuda et al., 1990, Nature, 346:561-564; Gerard et al., 1991, Biochem. J., 279:129-134; Munro et al., 1993, Nature, 365:61-65). CB1 is found primarily in the central nervous system, mainly in the cerebellum, hippocampus, and striatum (Tsou et al., 1998, Neurosci., 83:393-411), and has the highest density of any G-protein-coupled receptor in the brain (Herkenham et al., 1990, Proc. Natl. Acad. Sci. USA, 87:1932-1936). CB2 is found in peripheral tissues, including the spleen and macrophages.

CB1 has many cellular effects, such as a) inhibition of adenylate cyclase (Matsuda et al., 1990); b) modulation of Ca²⁺ and K⁺ channels (Mackie and Hille, 1992, Proc. Natl. Acad. Sci. USA, 89:3825-3829; Mackie et al., 1995, J. Neurosci., 15:6552-6561; Twitchell et al., 1997, J. Neurosci., 19:9271-9280); c) induction of the immediate-early gene Krox-24 (Bouaboula et al., 1995, J. Biol. Chem., 270:13973-13980); and d) activation of MAP kinases (Bouaboula et al., 1995, Biochem. J., 312:637-641). All of these actions involve the CB1 cannabinoid receptor coupling to pertussis toxin-sensitive G proteins, Gᵢ/Gₒ (Howlett, 1995, Ann. Rev. Pharmacol. Toxicol., 35:607-634). With respect to the modulation of Ca²⁺ channels, the CB1 cannabinoid receptor is unusual in that it is tonically active in the absence of agonist.

The CB1 cannabinoid receptor consists of 472 amino acids. The predicted structure of the protein is typical of a G-protein-coupled receptor, consisting of 1) an extracellular amino-terminal domain (amino acids 1-116) with putative glycosylation sites, 2) a transmembrane domain with seven helices, and 3) an intracellular carboxy-terminal domain (amino acids 401-472) (Matsuda et al., 1990, Nature, 346:561-564; Gerard et al., 1991, Biochem. J., 279: 129-134; and Munro et al., 1993, Nature, 365:61-65). There are two CB1 isoforms, CB1 and CB1A, that result from alternative splicing (Shire et al., 1995, J. Biol. Chem., 270:3726-3731; Rindaldi-Carmona et al., 1996, J. Pharmacol. Exp. Therapeu., 278:871-878). The CB1A isoform differs from the CB isoform in that the extracellular amino terminus of the CB1A isoform is 61 amino acids shorter than the extracellular amino terminus of the CB1 isoform. Structure-function correlation studies suggest that amino acids at positions 401-417 of CB1 are involved in Gᵢ/Gₒ binding, and that amino acids at positions 418-472 of CB1 are involved in receptor modulation (Howlett et al., 1998, Mol. Pharmacol., 53:504-510; Mukhopadhyay et al., 1999, Biochem., 38:3447-3455).

With regard to the receptor modulation, two domains in the intracellular C-terminal portion of CB1 mediate receptor desensitization and internalization (Hsieh et al., 1999, J. Neurochem., 73:493-501; Jin et al., 1999, J. Neurosci., 19:3773-3780). First, amino acids 460-463 of CB1 have been demonstrated to be essential for agonist-stimulated rapid receptor internalization via clathrin-coated pits (Hsieh et al., 1999, J. Neurochem., 73:493-501). Second, the CB1 agonist WIN55,212-2 evokes an increase in K⁺ conductance in an oocyte expression model, which desensitizes CB1 only in the presence of GRK3 and β-arrestin. Residues 418-439 in the C-terminal region of CB1 (especially the serine residues at positions 426 and 430) are important for this effect (Jin et al., 1999, J. Neurosci., 19:3773-3780).

Although it is known in the art that various molecules may act as agonists, antagonists, or inverse agonists of the CB1 cannabinoid receptor pathway (See, e.g., U.S. Pat. Nos. 5,747,524 and 6,100,259; Pan et al., 1998, Molecular Pharmacology, 54:1064-1072; Bouaboula et al., 1997, Biol. Chem., 272:22330-22339), it is not known which, if any, endogenous proteins affect the activity of the CB1 receptor in vivo. Therefore, what is needed in the art are unique polypeptides that can interact with the CB1 receptor and that can modulate the CB1 receptor pathway. Also needed are methods of modulating the CB1 receptor activity to achieve a desired result through the use of such unique polypeptides.

### SUMMARY OF THE INVENTION

This invention fulfills in part the need to identify new, unique polypeptides capable of affecting the cannabinoid receptor pathways. In particular, the present invention describes novel CB1 Receptor Interacting Proteins 1a and 1b (CRIP1a and CRIP1b) and CRIP1a and CRIP1b coding nucleic acids.

The present invention provides isolated nucleic acids encoding polypeptides that can interact with the CB1 receptor. The present invention also provides vectors comprising any one of the described nucleic acids.

The present invention further provides novel polypeptides capable of interacting with the CB1 receptor. The present invention also provides antibodies specific to these CB1 receptor-interacting polypeptides.

The present invention also provides pharmaceutical compositions useful in modulating the activity of a CB1 receptor, comprising any one of the described isolated nucleic acids and a pharmaceutical carrier.

The present invention provides methods for modulating the activity of a CB1 cannabinoid receptor, comprising administering to the CB1 cannabinoid receptor an effective amount of a composition comprising a CB1 receptor-interacting protein (CRIP), a CRIP coding nucleic acid, or fragments thereof. The present invention also provides methods for modulating the activity of a CB1 cannabinoid receptor, comprising administering to the CB1 cannabinoid receptor an effective amount of an antibody to a CB1 receptor-interacting protein. In particular, the present invention provides methods for modulating the activity of the CB1 cannabinoid receptor in a patient which may result in one or more effects selected from the group consisting of appetite stimulation, analgesia, euphoria, decreased tremor or spasticity associated with multiple sclerosis, attenuation of nausea and vomiting in cancer chemotherapy, reduction of intraocular pressure, decreased intestinal motility, and attenuation of aversive memories. The present invention also provides methods for modulating the activity of the CB1 cannabinoid receptor in a patient which may result in one or more effects selected from the group consisting of appetite suppression, decreased sedation, decreased alterations in cognition and memory, and decreased mood alterations. In one embodiment, the methods modulate the activity of CB1 in the glutamatergic neurons. In another embodiment, the methods for modulating the activity of CB1 are used for a patient that is suffering from epilepsy. In yet another embodiment, the methods are used for a patient that is suffering from a drug or alcohol addiction or post traumatic stress disorder.

The present invention further provides methods of screening for a modulator of the CB1 Receptor-Interacting Proteins 1a and 1b (CRIP1a and CRIP1b) activity on a CB1 cannabinoid receptor, comprising combining a CB1 cannabinoid receptor and a CRIP1a or CRIP1b polypeptide in the presence and absence of a putative modulator, and comparing the interaction between the CB1 cannabinoid receptor and the CRIP1a or CRIP1b polypeptide in the presence and absence of the putative modulator.

These and other embodiments of the invention will become apparent to one of skill in the art upon review of the description of the invention.

### BRIEF DESCRIPTION OF THE DRAWINGS

FIG. 1 is a diagram showing the structure of the CB1 cannabinoid receptor.

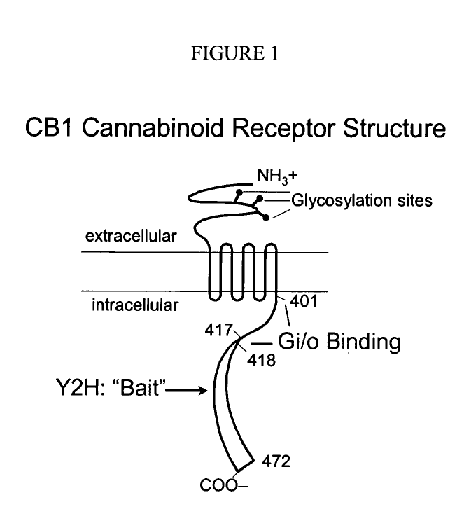

FIG. 2 is a schematic showing the structure of the human CRIP1a and CRIP1b genes which map to chromosome 2. The human ortholog of rat CRIP1a is the human retinoblastoma gene (gi:24308071). There is a 1.91 kb intron between exon 1 (dark shaded box) and exon 2 (light shaded box). Alternative splicing generates the third exon of human CRIP1b and that of the human retinoblastoma clone (medium shaded boxes).

FIG. 3 shows the homology of the rat CRIP1a amino acid sequence to the amino acid sequences of human CRIP1a and human CRIP1b. Differences between the two sequences being compared in each panel are indicated by the shaded regions. Panel A is an alignment of the rat CRIP1a amino acid sequence (SEQ ID NO:7) with the human CRIP1b amino acid sequence (SEQ ID NO:2). Panel B shows the rat CRIP1a amino acid sequence (SEQ ID NO:7) and the human CRIP1a amino acid sequence (SEQ ID NO:5). Each exon is shown on a separate line.

FIG. 4 is an alignment of the amino acid sequence of CRIP1b (hCB1-IP1) from human brain cells (SEQ ID NO:2) with regions from homologous proteins from retinoblastoma cells (SEQ ID NO:5), Macaque cells (SEQ ID NO:8), fetal kidney cells (SEQ ID NO:9), mouse cerebellar cells (SEQ ID NO:10), and mouse pituitary cells (SEQ ID NO: 11). A consensus sequence is also shown (SEQ ID NO: 12). Upper case letters indicate amino acids conserved between all sequences shown; lower case letters indicate amino acids which are conserved between a majority of the sequences shown.

FIG. 5 (A-E) shows the results of the yeast two-hybrid experiments with various portions of the CRIP1b polypeptide. The particular CRIP1b construct used and its results are depicted to the immediate left or right of the photograph of the plate. A “+” sign indicates that the yeast strains harboring the particular CRIP1b construct were blue, indicating an interaction between the CRIP1b and the CB1 construct. A “−” indicates that there was no interaction as shown by a lack of blue color.

FIG. 6 shows the results of the yeast two-hybrid experiments with various portions of the CB1 receptor. The results of a particular CB1 receptor construct used are depicted to the immediate right of the construct. A “+” sign indicates that the yeast strains harboring the particular CB1 receptor construct were blue, indicating an interaction between the CRIP1b construct and the CB1 C-terminal domain. A “−” indicates that there was no interaction as shown by a lack of blue color.

FIG. 7 is an SDS-PAGE gel showing the proteins eluted from the GST and GST fusion peptide columns of the affinity chromatography experiments. Lane 1 contains molecular weight markers; Lane 2 contains the eluate of the proteins in the CRIP1b lysate that bound to the hCB1-GST-glutathione sepharose column; and Lane 3 contains the eluate of the proteins in the CRIP1b lysate that bound to the glutathione sepharose only column.

FIG. 8 is a Western blot of an SDS-PAGE gel as in FIG. 9. The CRIP1b polypeptide was detected with an S-tag probe. Lane 1 contains the CRIP1b lysate prior to being passed through a column; Lane 2 contains the eluate of the proteins in the CRIP1b lysate that bound to the hCB1-GST-glutathione sepharose column; and Lane 3 contains the eluate of the proteins in the CRIP1b lysate that bound to the glutathione sepharose only column.

FIG. 9 shows the specificity of the polyclonal anti-CRIP1b antibody. Lane 1 is an SDS-PAGE gel stained with Coomassie blue stain and contains the bacterial lysate of E. coli cells induced to express CRIP1b. Lanes 2-7 are Western blots of SDS-PAGE gels. Lane 2, 3, and 4 are controls. Lane 2 contains lysate from cells that were transformed with the empty vector, and the blot was reacted with preimmune serum; Lane 3 contains lysate from cells that were transformed with the empty vector, and the blot was reacted with anti-CRIP1b antibody. Lane 4 contains the lysate from cells that were transformed with the CRIP1a or CRIP1b vector, and the blot was reacted with preimmune serum. Lane 5 contains the lysate from cells that were transformed with the CRIP1 a or CRIP1b vector, and the blot was reacted with anti-CRIP1b antibody. Lane 6 contains the lysate from cells that were transformed with the empty vector, and the blot was reacted with an S-tag probe. Lane 7 contains the lysate from cells that were transformed with the CRIP1 a or CRIP1b vector, and the blot was reacted with an S-tag probe.

FIG. 10 is a Western blot of an SDS-PAGE gel containing proteins from various human tissues. Approximate molecular weights are indicated to the right of the blot. Lane 1 contains proteins expressed in the lung. Lane 2 contains proteins expressed in the heart. Lane 3 contains proteins expressed in the small intestine. Lane 4 contains proteins expressed in the spleen. Lane 5 contains proteins expressed in the testis. Lane 6 contains proteins expressed in the stomach. Lane 7 contains proteins expressed in the brain.

FIG. 11 shows the analysis of CRIP1a expression in mouse brain. Panel A is a Western blot showing the immunostaining of a single band with the expected molecular mass for CRIP1a (˜18.5 kDa) in a cerebellar homogenate (lane 1) and a whole brain homogenate (lane 2). Preabsorption of the CRIP1a antisera with CRIP1a peptide antigen completely blocks the immunostaining of the band in both cerebellar homogenate (lane 3) and whole brain homogenate (lane 4). Panel B shows the immunocytochemical staining of the CRIP1a antibody in a saggital section of the mouse brain. The immunostaining seen was absent when the antiserum was pre-absorbed with the CRIP1a antigen peptide. The abbreviations in this figure denote the anterior olfactory nucleus (AON), cerebellum (Cb), caudate putamen (Cpu), neocortex (Cx), hippocampus (Hi), hypothalamus (Hy), interior colliculus (IC), lateral olfactory tract (lo), olfactory bulb (OB), superior colliculus (SC), substantia nigra pars reticulata (SNR), and thalamus (Th).

FIG. 12 is a graph showing the GIRK currents elicited by a step from −60 to −100 mV in the absence and presence of CP55,940, WIN 55,212-2, or Baclofen.

FIG. 13 is a photograph of an electrophoretic gel on which RT-PCR samples from different cell lines and primary neuronal cultures were electrophoresed. The results of these experiments show that CRIP1a (Row C), but not CRIP1b (Row B), has been detected in AtT20, HEK293, N18TG2, rat cerebellar granule neurons (CGN), and rat superior cervical ganglion (SCG). The CRIP1a product was found to be present only in samples that contained reverse transcriptase. Row A is a positive control (rig/S15).

FIG. 14 is a graph showing the effect of rat CRIP1a on the constitutive activity of CB1 receptors on Ca²⁺ channels. Coexpression of CRIP1a with the CB1 receptor significantly inhibited the effect of the inverse agonist SR141716, while expression of CRIP1a alone had no effect.

FIG. 15 is a schematic diagram illustrating the data from FIG. 14, showing that CRIP1a significantly decreased the effect of the CB1 inverse agonist SR141716 (“SR”) on the CB1 receptor.

FIG. 16 is a graph showing the effect of rat CRIP1a on the constitutive activity of CB1 receptors on Ca²⁺ channels. Coexpression of CRIP1a with the CB1 receptor showed no significant effect on the agonist-induced (WIN55,212-2) Ca²⁺ inhibition by the CB1 receptor.

FIG. 17 is a schematic diagram illustrating the data from FIG. 16, showing that CRIP1a has no effect on the agonist-induced (WIN55,212-2, “WIN”) Ca²⁺ inhibition by the CB1 receptor.

FIG. 18 is a graph showing the effect of rat CRIP1a on the constitutive activity of wild type CB1 receptor or the CB1-464 truncated receptor on Ca²⁺ channels. Coexpression of CRIP1a with the CB1-464 truncated receptor did not significantly inhibit effect of SR141716, suggesting that CRIP1a interacts with the distal 9 amino acids of CB1.

### DETAILED DESCRIPTION OF THE INVENTION

The present invention may be understood more readily by reference to the following detailed description of the preferred embodiments of the invention and the Examples included herein. However, before the present compounds, compositions, and methods are disclosed and described, it is to be understood that this invention is not limited to specific nucleic acids, specific polypeptides, specific cell types, specific host cells, specific conditions, or specific methods, etc., as such may, of course, vary, and the numerous modifications and variations therein will be apparent to those skilled in the art. It is also to be understood that the terminology used herein is for the purpose of describing specific embodiments only and is not intended to be limiting. It is further to be understood that unless specifically defined herein, the terminology used herein is to be given its traditional meaning as known in the relative art.

The present invention describes for the first time that the rat and Homo sapiens CRIP1a and CRIP1b polypeptides can interact with the CB1 cannabinoid receptor (CB1) and that the CRIP1a and CRIP1b polypeptides are useful in modulating the activity of CB1. As used herein, the terms “peptide,” “polypeptide,” and “protein” refer to a chain of at least four amino acids joined by peptide bonds. The chain may be linear, branched, circular, or combinations thereof. Accordingly, the present invention provides isolated CRIP1a and CRIP1b polypeptides. In preferred embodiments, the CRIP1b polypeptide is defined in SEQ ID NO:2, and the CRIP1a polypeptide is defined in SEQ ID NO:5 or SEQ ID NO:7.

The CRIP1a and CRIP1b polypeptides of the present invention are preferably produced by recombinant DNA techniques. For example, a nucleic acid molecule encoding the polypeptide is cloned into an expression vector, the expression vector is introduced into a host cell, and the CRIP1a or CRIP1b polypeptide is expressed in the host cell. The CRIP1a or CRIP1b polypeptide can then be isolated from the cells by an appropriate purification scheme using standard polypeptide purification techniques. For the purposes of the invention, the term “recombinant polynucleotide” refers to a polynucleotide that has been altered, rearranged, or modified by genetic engineering. Examples include any cloned polynucleotide, and polynucleotides that are linked or joined to heterologous sequences. The term “recombinant” does not refer to alterations to polynucleotides that result from naturally occurring events, such as spontaneous mutations. Alternative to recombinant expression, a CRIP1a or CRIP1b polypeptide, or peptide thereof, can be synthesized chemically using standard peptide synthesis techniques. Moreover, native CRIP1a or CRIP1b polypeptide can be isolated from cells (e.g., human brain cells), for example using an anti-CRIP1a or anti-CRIP1b polypeptide antibody.

As used herein, the term “nucleic acid” and “polynucleotide” refer to RNA or DNA that is linear or branched, single or double stranded, or a hybrid thereof. The term also encompasses RNA/DNA hybrids. These terms also encompass untranslated sequence located at both the 3′ and 5′ ends of the coding region of the gene: at least about 1000 nucleotides of sequence upstream from the 5′ end of the coding region and at least about 200 nucleotides of sequence downstream from the 3′ end of the coding region of the gene. Less common bases, such as inosine, 5-methylcytosine, 6-methyladenine, hypoxanthine, and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made. The antisense polynucleotides and ribozymes can consist entirely of ribonucleotides, or can contain mixed ribonucleotides and deoxyribonucleotides. The polynucleotides of the invention may be produced by any means, including genomic preparations, cDNA preparations, in vitro synthesis, RT-PCR, and in vitro or in vivo transcription.

An “isolated” nucleic acid or polynucleotide molecule is one that is substantially separated from other nucleic acid molecules, which are present in the natural source of the nucleic acid (i.e., sequences encoding other polypeptides). Preferably, an “isolated” nucleic acid is free of some of the sequences, which naturally flank the nucleic acid (i.e. sequences located at the 5′ and 3′ ends of the nucleic acid) in its naturally occurring replicon. For example, a cloned nucleic acid is considered isolated. In various embodiments, the isolated CRIP1a or CRIP1b nucleic acid molecule can contain less than about 5 kb, 4 kb, 3 kb, 2 kb, 1 kb, 0.5 kb, or 0.1 kb of nucleotide sequences which naturally flank the nucleic acid molecule in genomic DNA of the cell from which the nucleic acid is derived (e.g., a human or rat cell). A nucleic acid is also considered isolated if it has been altered by human intervention, or placed in a locus or location that is not its natural site. Moreover, an “isolated” nucleic acid molecule, such as a cDNA molecule, can be free from some of the other cellular material with which it is naturally associated, or culture medium when produced by recombinant techniques, or chemical precursors or other chemicals when chemically synthesized.

Specifically excluded from the definition of “isolated nucleic acids” are: naturally-occurring chromosomes (such as chromosome spreads), artificial chromosome libraries, genomic libraries, and cDNA libraries that exist either as an in vitro nucleic acid preparation or as a transfected/transformed host cell preparation, wherein the host cells are either an in vitro heterogeneous preparation or plated as a heterogeneous population of single colonies. Also specifically excluded are the above libraries wherein a specified nucleic acid makes up less than 5% of the number of nucleic acid inserts in the vector molecules. Further specifically excluded are whole cell genomic DNA or whole cell RNA preparations (including whole cell preparations that are mechanically sheared or enzymatically digested). Even further specifically excluded are the whole cell preparations found as either an in vitro preparation or as a heterogeneous mixture separated by electrophoresis wherein the nucleic acid of the invention has not further been separated from the heterologous nucleic acids in the electrophoresis medium (e.g., further separating by excising a single band from a heterogeneous band population in an agarose gel or nylon blot).

A nucleic acid molecule of the present invention, e.g., a nucleic acid molecule having a nucleotide sequence of SEQ ID NO:1, SEQ ID NO:3, SEQ ID NO:4, or SEQ ID NO:6, or a portion thereof, can be isolated using standard molecular biology techniques and the sequence information provided herein. For example, a CRIP1a or CRIP1b cDNA can be isolated from a cDNA library using all or portion of one of the sequences of SEQ ID NO:1, SEQ ID NO:3, SEQ ID NO:4, or SEQ ID NO:6. Moreover, a nucleic acid molecule encompassing all or a portion of one of the sequences of SEQ ID NO:1, SEQ ID NO:3, SEQ ID NO:4, or SEQ ID NO:6 can be isolated by the polymerase chain reaction (PCR) using oligonucleotide primers designed based upon this sequence. For example, mRNA can be isolated from a cell, and synthetic oligonucleotide primers for PCR amplification can be designed based upon one of the nucleotide sequences shown in SEQ ID NO:1, SEQ ID NO:3, SEQ ID NO:4, or SEQ ID NO:6. A nucleic acid molecule of the invention can be amplified using cDNA or, alternatively, genomic DNA, as a template and appropriate oligonucleotide primers according to standard PCR amplification techniques. The nucleic acid molecule so amplified can be cloned into an appropriate vector and characterized by DNA sequence analysis. Furthermore, oligonucleotides corresponding to a CRIP1a or CRIP1b nucleotide sequence can be prepared by standard synthetic techniques, e.g., using an automated DNA synthesizer.

In a preferred embodiment, an isolated nucleic acid molecule of the invention comprises one of the polynucleotide sequences shown in SEQ ID NO:1, SEQ ID NO:3, SEQ ID NO:4, or SEQ ID NO:6. Moreover, the nucleic acid molecule of the invention can comprise a portion of the coding region of one of the sequences in SEQ ID NO:1, SEQ ID NO:3, SEQ ID NO:4, or SEQ ID NO:6, for example, a fragment that can be used as a probe or primer or a fragment encoding a biologically active portion of a CRIP1a or CRIP1b. The nucleotide sequences determined from the cloning of the CRIP1a and CRIP1b genes from human and rat cells allow for the generation of probes and primers designed for use in identifying and cloning CRIP1a and CRIP1b homologs from other cell types and organisms.

As used herein, the term “biologically active portion of” a CRIP1a or CRIP1b is intended to include a portion, e.g., a domain/motif, of a CRIP1a or CRIP1b that participates in the interaction with CB1 and/or the modulation of CB1 constitutive activity. Biologically active portions of a CRIP1a or CRIP1b polypeptide include peptides comprising amino acid sequences derived from the amino acid sequence of a CRIP1a or CRIP1b, e.g., an amino acid sequence of SEQ ID NO:2, SEQ ID NO:5, or SEQ ID NO:7, or the amino acid sequence of a polypeptide identical to a CRIP1a or CRIP1b, which includes fewer amino acids than a full length CRIP1a or CRIP1b, or the full length polypeptide which is identical to a CRIP1a or CRIP1b, and exhibits at least one activity of a CRIP1a or CRIP1b. Typically, biologically active portions (e.g., peptides which are, for example, 5, 10, 15, 20, 30, 35, 36, 37, 38, 39, 40, 50, 100, or more amino acids in length) comprise a domain or motif with at least one activity of a CRIP1a or CRIP1b. As used herein, the term “CB1 activity” is intended to include, but is not limited to, the tonic inhibition of Ca²⁺ and K⁺ channels, the sequestering of G-proteins, the inhibition of adenylate cyclase, the activation of MAP kinases, and the induction of the immediate-early gene Krox-24. For the purposes of the present invention, modulation of CB1 activity refers to at least a 10% increase or decrease in the CB1 activity as compared to the CB1 activity in the absence of the CRIP1a or CRIP1b polypeptide or peptide. For the purposes of this invention, an increase in CB1 activity results in appetite stimulation, analgesia, euphoria, decreased tremor or spasticity associated with multiple sclerosis, attenuation of nausea and vomiting in cancer chemotherapy, reduction of intraocular pressure, decreased intestinal motility, and attenuation of aversive memories. Also for the purposes of this invention, a decrease in CB1 activity results in appetite suppression, decreased sedation, decreased alterations in cognition and memory, and decreased mood alterations.

The invention also provides CRIP1a and CRIP1b chimeric or fusion polypeptides. As used herein, a CRIP1a or CRIP1b “chimeric polypeptide” or “fusion polypeptide” comprises a CRIP1a or CRIP1b operatively linked to a non-CRIP1a or non-CRIP1b polypeptide. A CRIP1a or CRIP1b refers to a polypeptide having an amino acid sequence corresponding to a CRIP1a or CRIP1b, whereas a non-CRIP1a or non-CRIP1b refers to a polypeptide having an amino acid sequence corresponding to a polypeptide which is not substantially identical to the CRIP1a or CRIP1b polypeptide, e.g., a polypeptide that is different from the CRIP1a or CRIP1b and is derived from the same or a different organism. With respect to the fusion polypeptide, the term “operatively linked” is intended to indicate that the CRIP1a or CRIP1b and the non-CRIP1a or non-CRIP1b are fused to each other so that both sequences fulfill the proposed function attributed to the sequence used. The non-CRIP1a or non-CRIP1b can be fused to the N-terminus or C-terminus of the CRIP1a or CRIP1b. For example, in one embodiment, the fusion polypeptide is a GST-CRIP1a or GST-CRIP1b fusion polypeptide in which the CRIP1a or CRIP1b sequence is fused to the C-terminus of the GST sequence. Such fusion polypeptides can facilitate the purification of recombinant CRIP1a or CRIP1b polypeptides. In another embodiment, the fusion polypeptide is a CRIP1b polypeptide containing a heterologous signal sequence at its N-terminus. In certain host cells (e.g., mammalian host cells), expression and/or secretion of a CRIP1b polypeptide can be increased through use of a heterologous signal sequence.

Preferably, a CRIP1a or CRIP1b chimeric or fusion polypeptide of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and re-amplified to generate a chimeric gene sequence (See, for example, Current Protocols in Molecular Biology, Eds. Ausubel et al. John Wiley & Sons: 1992). Moreover, many expression vectors are commercially available that already encode a fusion moiety (e.g., a GST polypeptide). A CRIP1a or CRIP1b encoding nucleic acid can be cloned into such an expression vector such that the fusion moiety is linked in-frame to the CRIP1a or CRIP1b.

In addition to fragments and fusion polypeptides of the CRIP1a or CRIP1b polypeptides described herein, the present invention includes homologs and analogs of naturally occurring CRIP1a and CRIP1b polypeptides and CRIP1a and CRIP1b encoding nucleic acids in the same or other organisms. “Homologs” are defined herein as two nucleic acids or polypeptides that have similar or “identical,” nucleotide or amino acid sequences, respectively. Homologs include allelic variants, orthologs, paralogs, agonists, and antagonists of CRIP1a or CRIP1b polypeptides as defined hereafter. The term “homolog” further encompasses nucleic acid molecules that differ from one of the nucleotide sequences shown in SEQ ID NO:1, SEQ ID NO:3, SEQ ID NO:4, and SEQ ID NO:6 (and portions thereof) due to degeneracy of the genetic code and thus encode the same CRIP1 a or CRIP1b as that encoded by the nucleotide sequences shown in SEQ ID NO:1, SEQ ID NO:3, SEQ ID NO:4, and SEQ ID NO:6. As used herein, a “naturally occurring” CRIP1a or CRIP1b refers to a CRIP1a or CRIP1b amino acid sequence that occurs in nature. Preferably, a naturally occurring CRIP1a or CRIP1b comprises an amino acid sequence as defined in SEQ ID NO:2, SEQ ID NO:5, or SEQ ID NO:7.

An agonist of the CRIP1a or CRIP1b can retain substantially the same, or a subset, of the biological activities of the CRIP1a or CRIP1b. An antagonist of the CRIP1a or CRIP1b can inhibit one or more of the activities of the naturally occurring form of the CRIP1a or CRIP1b.

Nucleic acid molecules corresponding to natural allelic variants and analogs, orthologs, and paralogs of a CRIP1a or CRIP1b cDNA can be isolated based on their identity to the human or rat CRIP1a or CRIP1b nucleic acids described herein using CRIP1a or CRIP1b cDNAs, or a portion thereof, as a hybridization probe according to standard hybridization techniques under stringent hybridization conditions. In an alternative embodiment, homologs of the CRIP1a or CRIP1b can be identified by screening combinatorial libraries of mutants, e.g., truncation mutants, of the CRIP1a or CRIP1b for CRIP1a or CRIP1b agonist or antagonist activity. In one embodiment, a variegated library of CRIP1a or CRIP1b variants is generated by combinatorial mutagenesis at the nucleic acid level and is encoded by a variegated gene library. A variegated library of CRIP1a or CRIP1b variants can be produced by, for example, enzymatically ligating a mixture of synthetic oligonucleotides into gene sequences such that a degenerate set of potential CRIP1a or CRIP1b sequences is expressible as individual polypeptides, or alternatively, as a set of larger fusion polypeptides (e.g., for phage display) containing the set of CRIP1a or CRIP1b sequences therein. There are a variety of methods that can be used to produce libraries of potential CRIP1a or CRIP1b homologs from a degenerate oligonucleotide sequence. Chemical synthesis of a degenerate gene sequence can be performed in an automatic DNA synthesizer, and the synthetic gene is then ligated into an appropriate expression vector. Use of a degenerate set of genes allows for the provision, in one mixture, of all of the sequences encoding the desired set of potential CRIP1a or CRIP1b sequences. Methods for synthesizing degenerate oligonucleotides are known in the art (See, e.g., Narang, S. A., 1983, Tetrahedron 39:3; Itakura et al., 1984, Annu. Rev. Biochem. 53:323; Itakura et al., 1984, Science 198:1056; Ike et al., 1983, Nucleic Acid Res. 11:477).

In addition, libraries of fragments of the CRIP1a or CRIP1b coding regions can be used to generate a variegated population of CRIP1a or CRIP1b fragments for screening and subsequent selection of homologs of a CRIP1a or CRIP1b. In one embodiment, a library of coding sequence fragments can be generated by treating a double stranded PCR fragment of a CRIP1a or CRIP1b coding sequence with a nuclease under conditions wherein nicking occurs only about once per molecule, denaturing the double stranded DNA, renaturing the DNA to form double stranded DNA, which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal, and internal fragments of various sizes of the CRIP1a or CRIP1b.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of CRIP1a or CRIP1b homologs. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique that enhances the frequency of functional mutants in the libraries, can be used in combination with the screening assays to identify CRIP1a or CRIP1b homologs (Arkin and Yourvan, 1992, PNAS 89:7811-7815; Delgrave et al., 1993, Polypeptide Engineering 6(3):327-331). In another embodiment, cell based assays can be exploited to analyze a variegated CRIP1a or CRIP1b library, using methods well known in the art. The present invention further provides a method of identifying a novel CRIP1a or CRIP1b, comprising (a) raising a specific antibody response to a CRIP1a or CRIP1b, or a fragment thereof, as described herein; (b) screening putative CRIP1a or CRIP1b material with the antibody, wherein specific binding of the antibody to the material indicates the presence of a potentially novel CRIP1a or CRIP1b; and (c) analyzing the bound material in comparison to a known CRIP1a or CRIP1b, to determine its novelty.

As stated above, the present invention includes CRIP1a or CRIP1b polypeptides and homologs thereof. To determine the percent sequence identity of two amino acid sequences (e.g., one of the sequences of SEQ ID NO:2, SEQ ID NO:5, or SEQ ID NO:7, and a mutant form thereof), the sequences are aligned for optimal comparison purposes (e.g., gaps can be introduced in the sequence of one polypeptide for optimal alignment with the other polypeptide or nucleic acid). The amino acid residues at corresponding amino acid positions are then compared. When a position in one sequence (e.g., one of the sequences of SEQ ID NO:2, SEQ ID NO:5, or SEQ ID NO:7) is occupied by the same amino acid residue as the corresponding position in the other sequence (e.g., a mutant form of the sequence of SEQ ID NO:2, SEQ ID NO:5, or SEQ ID NO:7), then the molecules are identical at that position. The same type of comparison can be made between two nucleic acid sequences.

The percent sequence identity between the two sequences is a function of the number of identical positions shared by the sequences (i.e., percent sequence identity=numbers of identical positions/total numbers of positions ×100). Preferably, the isolated amino acid homologs included in the present invention are at least about 50-60%, preferably at least about 60-70%, and more preferably at least about 70-75%, 75-80%, 80-85%, 85-90%, or 90-95%, and most preferably at least about 96%, 97%, 98%, 99%, or more identical to an entire amino acid sequence shown in SEQ ID NO:2, SEQ ID NO:5, or SEQ ID NO:7. In yet another embodiment, the isolated amino acid homologs included in the present invention are at least about 50-60%, preferably at least about 60-70%, and more preferably at least about 70-75%, 75-80%, 80-85%, 85-90%, or 90-95%, and most preferably at least about 96%, 97%, 98%, 99%, or more identical to an entire amino acid sequence encoded by a nucleic acid sequence shown in SEQ ID NO:2, SEQ ID NO:5, or SEQ ID NO:7. In other embodiments, the CRIP1a or CRIP1b amino acid homologs have sequence identity over at least 15 contiguous amino acid residues, more preferably at least 25 contiguous amino acid residues, and most preferably at least 35 contiguous amino acid residues of SEQ ID NO:2, SEQ ID NO:5, or SEQ ID NO:7. In another embodiment, the homologs of the present invention are preferably at least about 60-70%, and more preferably at least about 70-75%, 75-80%, 80-85%, 85-90%, or 90-95%, and most preferably at least about 96%, 97%, 98%, 99%, or more identical through Exon 1 and/or Exon 2 of SEQ ID NO:2, SEQ ID NO:5, or SEQ ID NO:7.

In another preferred embodiment, an isolated nucleic acid homolog of the invention comprises a nucleotide sequence which is at least about 40-60%, preferably at least about 60-70%, more preferably at least about 70-75%, 75-80%, 80-85%, 85-90%, or 90-95%, and even more preferably at least about 95%, 96%, 97%, 98%, 99%, or more identical to a nucleotide sequence shown in SEQ ID NO:1, SEQ ID NO:3, SEQ ID NO:4, or SEQ ID NO:6, or to a portion comprising at least 60 consecutive nucleotides thereof. In one embodiment, the CRIP1a or CRIP1b homolog nucleotide sequence is about 50% identical to a nucleotide sequence shown in SEQ ID NO:1, SEQ ID NO:3, SEQ ID NO:4, or SEQ ID NO:6. The preferable length of sequence comparison for nucleic acids is at least 75 nucleotides, more preferably at least 100 nucleotides, and most preferably the entire length of the coding region. It is even more preferable that the nucleic acid homologs encode proteins having homology with SEQ ID NO:2, SEQ ID NO:5, or SEQ ID NO:7 over Exons 1 and 2 (corresponding to positions 1-110 of SEQ ID NO:2).

It is further preferred that the isolated nucleic acid homolog of the invention encodes a CRIP1a or CRIP1b, or portion thereof, that is at least 70% identical to an amino acid sequence of SEQ ID NO:2, SEQ ID NO:5, or SEQ ID NO:7, and that functions by interacting with the CB1 receptor and modulating its activity.

For the purposes of the invention, the percent sequence identity between two nucleic acid or polypeptide sequences is determined using the Vector NTI 6.0 (PC) software package (InforMax, 7600 Wisconsin Ave., Bethesda, Md. 20814). A gap opening penalty of 15 and a gap extension penalty of 6.66 are used for determining the percent identity of two nucleic acids. A gap opening penalty of 10 and a gap extension penalty of 0.1 are used for determining the percent identity of two polypeptides. All other parameters are set at the default settings. For purposes of a multiple alignment (Clustal W algorithm), the gap opening penalty is 10, and the gap extension penalty is 0.05 with blosum62 matrix. It is to be understood that for the purposes of determining sequence identity when comparing a DNA sequence to an RNA sequence, a thymidine nucleotide is equivalent to a uracil nucleotide.

In another aspect, the invention provides an isolated nucleic acid comprising a polynucleotide that hybridizes to the polynucleotide of SEQ ID NO:1, SEQ ID NO:3, SEQ ID NO:4, or SEQ ID NO:6 under stringent conditions. More particularly, an isolated nucleic acid molecule of the invention is at least 15 nucleotides in length and hybridizes under stringent conditions to the nucleic acid molecule comprising a nucleotide sequence of SEQ ID NO:1, SEQ ID NO:3, SEQ ID NO:4, or SEQ ID NO:6. In other embodiments, the nucleic acid is at least 30, 50, 100, 250, or more nucleotides in length. Preferably, an isolated nucleic acid homolog of the invention comprises a nucleotide sequence which hybridizes under highly stringent conditions to the nucleotide sequence shown in SEQ ID NO:1, SEQ ID NO:3, SEQ ID NO:4, or SEQ ID NO:6 and encodes a polypeptide that functions by interacting with the CB1 receptor and modulating its activity.

As used herein with regard to hybridization for DNA to a DNA blot, the term “stringent conditions” refers to hybridization at 65° C. in a 6×SSC solution. In another embodiment, the term “stringent conditions” refers to hybridization overnight at 60° C. in 10× Denhart's solution, 6× SSC, 0.5% SDS, and 100 μg/ml denatured salmon sperm DNA. Blots are washed sequentially at 62° C. for 30 minutes each time in 3× SSC/0.1% SDS, followed by 1× SSC/0.1% SDS, and finally 0.1× SSC/0.1% SDS. As also used herein, “highly stringent conditions” refers to hybridization overnight at 65° C. in 10× Denhart's solution, 6× SSC, 0.5% SDS, and 100 μg/ml denatured salmon sperm DNA. Blots are washed sequentially at 65° C. for 30 minutes each time in 3× SSC/0.1% SDS, followed by 1× SSC/0.1% SDS, and finally 0.1× SSC/0.1% SDS. Methods for nucleic acid hybridizations are described in Meinkoth and Wahl, 1984, Anal. Biochem. 138:267-284; Current Protocols in Molecular Biology, Chapter 2, Ausubel et al. Eds., Greene Publishing and Wiley-Interscience, New York, 1995; and Tijssen, 1993, Laboratory Techniques in Biochemistry and Molecular Biology: Hybridization with Nucleic Acid Probes, Part I, Chapter 2, Elsevier, New York, 1993. Preferably, an isolated nucleic acid molecule of the invention that hybridizes under stringent or highly stringent conditions to a sequence of SEQ ID NO:1, SEQ ID NO:3, SEQ ID NO:4, or SEQ ID NO:6 corresponds to a naturally occurring nucleic acid molecule. As used herein, a “naturally occurring” nucleic acid molecule refers to an RNA or DNA molecule having a nucleotide sequence that occurs in nature (e.g., encodes a natural polypeptide). In one embodiment, the nucleic acid encodes a naturally occurring human or rat CRIP1a or CRIP1b polypeptide.

Using the above-described methods, and others known to those of skill in the art, one of ordinary skill in the art can isolate homologs of the CRIP1a and CRIP1 γ polypeptides comprising amino acid sequences shown in SEQ ID NO:2, SEQ ID NO:5, or SEQ ID NO:7. One subset of these homologs is allelic variants. As used herein, the term “allelic variant” refers to a nucleotide sequence containing polymorphisms that lead to changes in the amino acid sequence of a CRIP1a or CRIP1b and that exist within a natural population. Such natural allelic variations can typically result in 1-5% variance in a CRIP1a or CRIP1b nucleic acid. Allelic variants can be identified by sequencing the nucleic acid sequence of interest in a number of different organisms, which can be readily carried out by using hybridization probes to identify the same CRIP1a or CRIP1b genetic locus in those organisms. Any and all such nucleic acid variations and resulting amino acid polymorphisms or variations in a CRIP1a or CRIP1b, that are the result of natural allelic variation and that do not alter the functional activity of a CRIP1a or CRIP1b, are intended to be within the scope of the invention.

Moreover, nucleic acid molecules encoding CRIP1a or CRIP1b polypeptides from the same or other species such as CRIP1a or CRIP1b analogs, orthologs, and paralogs, are intended to be within the scope of the present invention. As used herein, the term “analogs” refers to two nucleic acids that have the same or similar function, but that have evolved separately in unrelated organisms. As used herein, the term “orthologs” refers to two nucleic acids from different species, but that have evolved from a common ancestral gene by speciation. Normally, orthologs encode polypeptides having the same or similar functions. As also used herein, the term “paralogs” refers to two nucleic acids that are related by duplication within a genome. Paralogs usually have different functions, but these functions may be related (Tatusov, R. L. et al., 1997, Science 278(5338):631-637). Analogs, orthologs, and paralogs of a naturally occurring CRIP1a or CRIP1b can differ from the naturally occurring CRIP1a or CRIP1b by post-translational modifications, by amino acid sequence differences, or by both. Post-translational modifications include in vivo and in vitro chemical derivatization of polypeptides, e.g., acetylation, carboxylation, phosphorylation, or glycosylation, and such modifications may occur during polypeptide synthesis or processing or following treatment with isolated modifying enzymes. In particular, orthologs of the invention will generally exhibit at least 80-85%, more preferably, 85-90% or 90-95%, and most preferably 95%, 96%, 97%, 98%, or even 99% identity, or 100% sequence identity, with all or part of a naturally occurring CRIP1a or CRIP1b amino acid sequence, and will exhibit a function similar to an CRIP1a or CRIP1b. Preferably, a CRIP1a or CRIP1b ortholog of the present invention functions by interacting with the CB1 receptor and modulating its activity. In one embodiment, the CRIP1a or CRIP1b orthologs cause a decrease in CB1-mediated tonic inhibition of Ca²⁺ channels. In another embodiment, the CRIP1a or CRIP1b orthologs cause a decrease in CB1-mediated sequestering of G-proteins.

In addition to naturally-occurring variants of a CRIP1a or CRIP1b sequence that may exist in the population, the skilled artisan will further appreciate that changes can be introduced by mutation into a nucleotide sequence of SEQ ID NO:1, SEQ ID NO:3, SEQ ID NO:4, or SEQ ID NO:6 thereby leading to changes in the amino acid sequence of the encoded CRIP1a or CRIP1b, without altering the functional activity of the CRIP1a or CRIP1b. For example, nucleotide substitutions leading to amino acid substitutions at “non-essential” amino acid residues can be made in a sequence of SEQ ID NO:2, SEQ ID NO:5, or SEQ ID NO:7. A “non-essential” amino acid residue is a residue that can be altered from the wild-type sequence of one of the CRIP1a or CRIP1b polypeptides without altering the activity of said CRIP1a or CRIP1b, whereas an “essential” amino acid residue is required for CRIP1a or CRIP1b activity. Other amino acid residues, however, (e.g., those that are not conserved or only semi-conserved in the domain having CRIP1a or CRIP1b activity) may not be essential for activity and thus are likely to be amenable to alteration without altering CRIP1a or CRIP1b activity.

Accordingly, another aspect of the invention pertains to nucleic acid molecules encoding CRIP1a or CRIP1b polypeptides that contain changes in amino acid residues that are not essential for CRIP1a or CRIP1b activity. Such CRIP1a or CRIP1b polypeptides differ in amino acid sequence from a sequence contained in SEQ ID NO:2, SEQ ID NO:5, or SEQ ID NO:7, yet retain at least one of the CRIP1a or CRIP1b activities described herein. In one embodiment, the isolated nucleic acid molecule comprises a nucleotide sequence encoding a polypeptide, wherein the polypeptide comprises an amino acid sequence at least about 50% identical to an amino acid sequence of SEQ ID NO:2, SEQ ID NO:5, or SEQ ID NO:7. Preferably, the polypeptide encoded by the nucleic acid molecule is at least about 50-60% identical to one of the sequences of SEQ ID NO:2, SEQ ID NO:5, or SEQ ID NO:7, more preferably at least about 60-70% identical to one of the sequences of SEQ ID NO:2, SEQ ID NO:5, or SEQ ID NO:7, even more preferably at least about 70-75%, 75-80%, 80-85%, 85-90%, or 90-95% identical to one of the sequences of SEQ ID NO:2, SEQ ID NO:5, or SEQ ID NO:7, and most preferably at least about 96%, 97%, 98%, or 99% identical to one of the sequences of SEQ ID NO:2, SEQ ID NO:5, or SEQ ID NO:7. The preferred CRIP1a or CRIP1b homologs of the present invention preferably modulate CB1 activity.

An isolated nucleic acid molecule encoding a CRIP1a or CRIP1b having sequence identity with a polypeptide sequence of SEQ ID NO:2, SEQ ID NO:5, or SEQ ID NO:7 can be created by introducing one or more nucleotide substitutions, additions or deletions into a nucleotide sequence of SEQ ID NO:1, SEQ ID NO:3, SEQ ID NO:4, or SEQ ID NO:6, such that one or more amino acid substitutions, additions, or deletions are introduced into the encoded polypeptide. Mutations can be introduced into one of the sequences of SEQ ID NO:1, SEQ ID NO:3, SEQ ID NO:4, or SEQ ID NO:6 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain.

Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine). Thus, a predicted nonessential amino acid residue in a CRIP1a or CRIP1b is preferably replaced with another amino acid residue from the same side chain family. Alternatively, in another embodiment, mutations can be introduced randomly along all or part of a CRIP1a or CRIP1b coding sequence, such as by saturation mutagenesis, and the resultant mutants can be screened for a CRIP1a or CRIP1b activity described herein to identify mutants that retain CRIP1a or CRIP1b activity. Following mutagenesis of one of the sequences of SEQ ID NO:1, SEQ ID NO:3, SEQ ID NO:4, or SEQ ID NO:6, the encoded polypeptide can be expressed recombinantly and the activity of the polypeptide can be determined.

Additionally, optimized CRIP1a or CRIP1b nucleic acids can be created. Preferably, an optimized CRIP1a or CRIP1b nucleic acid encodes a CRIP1a or CRIP1b that binds to a CB1 receptor and modulates its activity. As used herein, “optimized” refers to a nucleic acid that is genetically engineered to increase its expression in a given organism. To provide optimized CRIP1a or CRIP1b nucleic acids, the DNA sequence of the gene can be modified to 1) comprise codons preferred by highly expressed genes in the organism; 2) comprise an A+T content in nucleotide base composition to that substantially found in the organism; 3) form an initiation sequence for that organism; or 4) to eliminate sequences that cause destabilization, inappropriate polyadenylation, degradation and termination of RNA, or that form secondary structure hairpins or RNA splice sites. Increased expression of CRIP1a or CRIP1b nucleic acids in an organism can be achieved by utilizing the distribution frequency of codon usage in a particular organism.

As used herein, “frequency of preferred codon usage” refers to the preference exhibited by a specific host cell in usage of nucleotide codons to specify a given amino acid. To determine the frequency of usage of a particular codon in a gene, the number of occurrences of that codon in the gene is divided by the total number of occurrences of all codons specifying the same amino acid in the gene. Similarly, the frequency of preferred codon usage exhibited by a host cell can be calculated by averaging frequency of preferred codon usage in a large number of genes expressed by the host cell. It is preferable that this analysis be limited to genes that are highly expressed by the host cell. The percent deviation of the frequency of preferred codon usage for a synthetic gene from that employed by a host cell is calculated first by determining the percent deviation of the frequency of usage of a single codon from that of the host cell followed by obtaining the average deviation over all codons. As defined herein, this calculation includes unique codons (i.e., ATG and TGG). In general terms, the overall average deviation of the codon usage of an optimized gene from that of a host cell is calculated using the equation 1A=n=1Z Xₙ−Yₙ Xₙ times 100 Z where Xₙ=frequency of usage for codon n in the host cell; Yₙ=frequency of usage for codon n in the synthetic gene; n represents an individual codon that specifies an amino acid; and the total number of codons is Z. The overall deviation of the frequency of codon usage, A, for all amino acids should preferably be less than about 25%, and more preferably less than about 10%.

Hence, a CRIP1a or CRIP1b nucleic acid can be optimized such that its distribution frequency of codon usage deviates, preferably, no more than 25% from that of highly expressed genes in that organism and, more preferably, no more than about 10%. In addition, consideration is given to the percentage G+C content of the degenerate third base.

In addition to the nucleic acid molecules encoding the CRIP1a or CRIP1b polypeptides described above, another aspect of the invention pertains to isolated nucleic acid molecules that are antisense thereto. Antisense polynucleotides are thought to inhibit gene expression of a target polynucleotide by specifically binding the target polynucleotide and interfering with transcription, splicing, transport, translation, and/or stability of the target polynucleotide. Methods are described in the prior art for targeting the antisense polynucleotide to the chromosomal DNA, to a primary RNA transcript, or to a processed mRNA. Preferably, the target regions include splice sites, translation initiation codons, translation termination codons, and other sequences within the open reading frame.

The term “antisense,” for the purposes of the invention, refers to a nucleic acid comprising a polynucleotide that is sufficiently complementary to all or a portion of a gene, primary transcript, or processed mRNA, so as to interfere with expression of the endogenous gene. “Complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two polynucleotides may hybridize to each other even if they are not completely complementary to each other, provided that each has at least one region that is substantially complementary to the other. The term “antisense nucleic acid” includes single stranded RNA as well as double-stranded DNA expression cassettes that can be transcribed to produce an antisense RNA. “Active” antisense nucleic acids are antisense RNA molecules that are capable of selectively hybridizing with a primary transcript or mRNA encoding a polypeptide having at least 80% sequence identity with the polypeptide of SEQ ID NO:2, SEQ ID NO:5, or SEQ ID NO:7.

The antisense nucleic acid can be complementary to an entire CRIP1a or CRIP1b coding strand, or to only a portion thereof. In one embodiment, an antisense nucleic acid molecule is antisense to a “coding region” of the coding strand of a nucleotide sequence encoding a CRIP1a or CRIP1b. The term “coding region” refers to the region of the nucleotide sequence comprising codons that are translated into amino acid residues. In another embodiment, the antisense nucleic acid molecule is antisense to a “noncoding region” of the coding strand of a nucleotide sequence encoding a CRIP1a or CRIP1b. The term “noncoding region” refers to 5′ and 3′ sequences that flank the coding region that are not translated into amino acids (i.e., also referred to as 5′ and 3′ untranslated regions). The antisense nucleic acid molecule can be complementary to the entire coding region of CRIP1a or CRIP1b mRNA, but more preferably is an oligonucleotide that is antisense to only a portion of the coding or noncoding region of CRIP1a or CRIP1b mRNA. For example, the antisense oligonucleotide can be complementary to the region surrounding the translation start site of CRIP1a or CRIP1b mRNA. An antisense oligonucleotide can be, for example, about 5, 10, 15, 20, 25, 30, 35, 40, 45, or 50 nucleotides in length. Typically, the antisense molecules of the present invention comprise an RNA having 60-100% sequence identity with at least 14 consecutive nucleotides of SEQ ID NO:1, SEQ ID NO:3, SEQ ID NO:4, or SEQ ID NO:6, or a polynucleotide encoding a polypeptide of SEQ ID NO:2, SEQ ID NO:5, or SEQ ID NO:7. Preferably, the sequence identity will be at least 70%, more preferably at least 75%, 80%, 85%, 90%, 95%, or 98%, and most preferably 99%.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al., 1987, Nucleic Acids. Res. 15:6625-6641). The antisense nucleic acid molecule can also comprise a 2′-o-methylribonucleotide (Inoue et al., 1987, Nucleic Acids Res. 15:6131-6148) or a chimeric RNA-DNA analogue (Inoue et al., 1987, FEBS Lett. 215:327-330).

The antisense nucleic acid molecules of the invention are typically administered to a cell or generated in situ such that they hybridize with or bind to cellular mRNA and/or genomic DNA encoding a CRIP1a or CRIP1b to thereby inhibit expression of the polypeptide, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. The antisense molecule can be modified such that it specifically binds to a receptor or an antigen expressed on a selected cell surface, e.g., by linking the antisense nucleic acid molecule to a peptide or an antibody which binds to a cell surface receptor or antigen. The antisense nucleic acid molecule can also be delivered to cells using the vectors described herein. To achieve sufficient intracellular concentrations of the antisense molecules, vector constructs in which the antisense nucleic acid molecule is placed under the control of a strong prokaryotic, viral, or eukaryotic (including plant) promoter are preferred.

As an alternative to antisense polynucleotides, ribozymes, sense polynucleotides, or double stranded RNA (dsRNA) can be used to reduce expression of a CRIP1a or CRIP1b polypeptide. As used herein, the term “ribozyme” refers to a catalytic RNA-based enzyme with ribonuclease activity that is capable of cleaving a single-stranded nucleic acid, such as an mRNA, to which it has a complementary region. Ribozymes (e.g., hammerhead ribozymes described in Haselhoff and Gerlach, 1988, Nature 334:585-591) can be used to catalytically cleave CRIP1b mRNA transcripts to thereby inhibit translation of CRIP1b mRNA. A ribozyme having specificity for a CRIP1a or CRIP1b-encoding nucleic acid can be designed based upon the nucleotide sequence of a CRIP1a or CRIP1b cDNA, as disclosed herein (i.e., SEQ ID NO:1, SEQ ID NO:4, or SEQ ID NO:6) or on the basis of a heterologous sequence to be isolated according to methods taught in this invention. For example, a derivative of a Tetrahymena L-19 IVS RNA can be constructed in which the nucleotide sequence of the active site is complementary to the nucleotide sequence to be cleaved in a CRIP1a or CRIP1b-encoding mRNA. See, e.g., U.S. Pat. Nos. 4,987,071 and 5,116,742 to Cech et al. Alternatively, CRIP1b mRNA can be used to select a catalytic RNA having a specific ribonuclease activity from a pool of RNA molecules. See, e.g., Bartel, D. and Szostak, J. W., 1993, Science 261:1411-1418. In preferred embodiments, the ribozyme will contain a portion having at least 7, 8, 9, 10, 12, 14, 16, 18, or 20 nucleotides, and more preferably 7 or 8 nucleotides, that have 100% complementarity to a portion of the target RNA. Methods for making ribozymes are known to those skilled in the art. See, e.g., U.S. Pat. Nos. 6,025,167; 5,773,260; and 5.496,698.

The term “dsRNA,” as used herein, refers to RNA hybrids comprising two strands of RNA. The dsRNAs can be linear or circular in structure. In a preferred embodiment, dsRNA is specific for a polynucleotide encoding either the polypeptide of SEQ ID NO:2, SEQ ID NO:5, or SEQ ID NO:7, or a polypeptide having at least 80% sequence identity with a polypeptide of SEQ ID NO:2, SEQ ID NO:5, or SEQ ID NO:7. The hybridizing RNAs may be substantially or completely complementary. By “substantially complementary,” is meant that when the two hybridizing RNAs are optimally aligned using the BLAST program as described above, the hybridizing portions are at least 95% complementary. Preferably, the dsRNA will be at least 100 base pairs in length. Typically, the hybridizing RNAs will be of identical length with no over hanging 5′ or 3′ ends and no gaps. However, dsRNAs having 5′ or 3′ overhangs of up to 100 nucleotides may be used in the methods of the invention.

The dsRNA may comprise ribonucleotides, ribonucleotide analogs such as 2′-O-methyl ribosyl residues, or combinations thereof. See, e.g., U.S. Pat. Nos. 4,130,641 and 4,024,222. A dsRNA polyriboinosinic acid:polyribocytidylic acid is described in U.S. Pat. No. 4,283,393. Methods for making and using dsRNA are known in the art. One method comprises the simultaneous transcription of two complementary DNA strands, either in vivo, or in a single in vitro reaction mixture. See, e.g., U.S. Pat. No. 5,795,715. In one embodiment, dsRNA can be introduced into a host cell directly by standard transformation procedures. Alternatively, dsRNA can be expressed in a host cell by transcribing two complementary RNAs.

Other methods for the inhibition of endogenous gene expression, such as triple helix formation (Moser et al., 1987, Science 238:645-650 and Cooney et al., 1988, Science 241:456-459) and co-suppression (Napoli et al., 1990, The Plant Cell 2:279-289) are known in the art. Partial and full-length cDNAs have been used for the co-suppression of endogenous plant genes. See, e.g., U.S. Pat. Nos. 4,801,340, 5,034,323, 5,231,020, and 5,283,184; Van der Kroll et al., 1990, The Plant Cell 2:291-299; Smith et al., 1990, Mol. Gen. Genetics 224:477-481; and Napoli et al., 1990, The Plant Cell 2:279-289.

For sense suppression, it is believed that introduction of a sense polynucleotide blocks transcription of the corresponding target gene. The sense polynucleotide will have at least 65% sequence identity with the target gene or RNA. Preferably, the percent identity is at least 80%, 90%, 95%, or more. The introduced sense polynucleotide need not be full length relative to the target gene or transcript. Preferably, the sense polynucleotide will have at least 65% sequence identity with at least 100 consecutive nucleotides of SEQ ID NO:1, SEQ ID NO:3, SEQ ID NO:4, or SEQ ID NO:6. The regions of identity can comprise introns and/or exons and untranslated regions. The introduced sense polynucleotide may be present in the host cell transiently, or may be stably integrated into a host chromosome or extrachromosomal replicon.

Alternatively, CRIP1a or CRIP1b gene expression can be inhibited by targeting nucleotide sequences complementary to the regulatory region of a CRIP1a or CRIP1b nucleotide sequence (e.g., a CRIP1a or CRIP1b promoter and/or enhancer) to form triple helical structures that prevent transcription of a CRIP1a or CRIP1b gene in target cells. See generally, Helene, C., 1991, Anticancer Drug Des. 6(6):569-84; Helene, C. et al., 1992, Ann. N.Y. Acad. Sci. 660:27-36; and Maher, L. J., 1992, Bioassays 14(12):807-15.

In addition to the CRIP1a or CRIP1b nucleic acids and polypeptides described above, the present invention encompasses these nucleic acids and polypeptides attached to a moiety. These moieties include, but are not limited to, detection moieties, hybridization moieties, purification moieties, delivery moieties, reaction moieties, binding moieties, and the like. A typical group of nucleic acids having moieties attached are probes and primers. Probes and primers typically comprise a substantially isolated oligonucleotide. The oligonucleotide typically comprises a region of nucleotide sequence that hybridizes under stringent conditions to at least about 12, preferably about 25, more preferably about 40, 50, or 75 consecutive nucleotides of a sense strand of one of the sequences set forth in SEQ ID NO:1, SEQ ID NO:3, SEQ ID NO:4, or SEQ ID NO:6; an anti-sense sequence of one of the sequences set forth in SEQ ID NO:1, SEQ ID NO:3, SEQ ID NO:4, or SEQ ID NO:6; or naturally occurring mutants thereof. Primers based on a nucleotide sequence of SEQ ID NO:1, SEQ ID NO:3, SEQ ID NO:4, or SEQ ID NO:6 can be used in PCR reactions to clone CRIP1a or CRIP1b homologs. Probes based on the CRIP1a or CRIP1b nucleotide sequences can be used to detect transcripts or genomic sequences encoding the same or substantially identical polypeptides. In preferred embodiments, the probe further comprises a label group attached thereto, e.g. the label group can be a radioisotope, a fluorescent compound, an enzyme, or an enzyme co-factor. Such probes can be used as a part of a genomic marker test kit for identifying cells which express a CRIP1a or CRIP1b, such as by measuring a level of a CRIP1a or CRIP1b-encoding nucleic acid, in a sample of cells, e.g., detecting CRIP1b mRNA levels or determining whether a genomic CRIP1a or CRIP1b gene has been mutated or deleted.

In particular, a useful method to ascertain the level of transcription of the gene (an indicator of the amount of mRNA available for translation to the gene product) is to perform a Northern blot (For reference, see, for example, Ausubel et al., 1988, Current Protocols in Molecular Biology, Wiley: New York). The information from a Northern blot at least partially demonstrates the degree of transcription of the transformed gene. Total cellular RNA can be prepared from cells, tissues, or organs by several methods, all well-known in the art, such as that described in Bormann, E. R. et al., 1992, Mol. Microbiol. 6:317-326. To assess the presence or relative quantity of polypeptide translated from this mRNA, standard techniques, such as a Western blot, may be employed. These techniques are well known to one of ordinary skill in the art. (See, for example, Ausubel et al., 1988, Current Protocols in Molecular Biology, Wiley: New York).

The invention further provides an isolated recombinant expression vector comprising a CRIP1a or CRIP1b nucleic acid as described above, wherein expression of the nucleic acid in a host cell results in modulation of CB1 receptor activity as compared to a wild type variety of the host cell. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses, and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. As used herein with respect to a recombinant expression vector, “operatively linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers, and other expression control elements (e.g., polyadenylation signals). Such regulatory sequences are described, for example, in Goeddel, Gene Expression Technology: Methods in Enzymology 185, Academic Press, San Diego, Calif. (1990) and Gruber and Crosby, in: Methods in Plant Molecular Biology and Biotechnology, eds. Glick and Thompson, Chapter 7, 89-108, CRC Press: Boca Raton, Fla., including the references therein. Regulatory sequences include those that direct constitutive expression of a nucleotide sequence in many types of host cells and those that direct expression of the nucleotide sequence only in certain host cells or under certain conditions. It will be appreciated by those skilled in the art that the design of the expression vector can depend on such factors as the choice of the host cell to be transformed, the level of expression of polypeptide desired, etc. The expression vectors of the invention can be introduced into host cells to thereby produce polypeptides or peptides, including fusion polypeptides or peptides, encoded by nucleic acids as described herein (e.g., CRIP1a or CRIP1b polypeptides, mutant forms of CRIP1a or CRIP1b polypeptides, fusion polypeptides, etc.).

The recombinant expression vectors of the invention can be designed for expression of CRIP1a or CRIP1b polypeptides in prokaryotic or eukaryotic cells. For example, CRIP1a or CRIP1b genes can be expressed in bacterial cells such as C. glutamicum, insect cells (using baculovirus expression vectors), yeast and other fungal cells (See Romanos, M. A. et al., 1992, Foreign gene expression in yeast: a review, Yeast 8:423-488; van den Hondel, C. A. M. J. J. et al., 1991, Heterologous gene expression in filamentous fungi, in: More Gene Manipulations in Fungi, J. W. Bennet & L. L. Lasure, eds., p. 396-428: Academic Press: San Diego; and van den Hondel, C. A. M. J. J. & Punt, P. J., 1991, Gene transfer systems and vector development for filamentous fungi, in: Applied Molecular Genetics of Fungi, Peberdy, J. F. et al., eds., p. 1-28, Cambridge University Press: Cambridge), algae (Falciatore et al., 1999, Marine Biotechnology 1(3):239-251), ciliates of the types: Holotrichia, Peritrichia, Spirotrichia, Suctoria, Tetrahymena, Paramecium, Colpidium, Glaucoma, Platyophrya, Potomacus, Pseudocohnilembus, Euplotes, Engelmaniella, and Stylonychia, especially of the genus Stylonychia lemnae with vectors following a transformation method as described in PCT Application No. WO 98/01572, and multicellular plant cells (See Schmidt, R. and Willmitzer, L., 1988, High efficiency Agrobacterium tumefaciens-mediated transformation of Arabidopsis thaliana leaf and cotyledon explants, Plant Cell Rep. 583-586; Plant Molecular Biology and Biotechnology, C Press, Boca Raton, Fla., chapter 6/7, S. 71-119 (1993); F. F. White, B. Jenes et al., Techniques for Gene Transfer, in: Transgenic Plants, Vol. 1, Engineering and Utilization, eds. Kung und R. Wu, 128-43, Academic Press: 1993; Potrykus, 1991, Annu. Rev. Plant Physiol. Plant Molec. Biol. 42:205-225 and references cited therein), or mammalian cells. Suitable host cells are discussed further in Goeddel, Gene Expression Technology: Methods in Enzymology 185, Academic Press: San Diego, Calif. (1990). Alternatively, the recombinant expression vector can be transcribed and translated in vitro, for example using T7 promoter regulatory sequences and T7 polymerase.

Expression of polypeptides in prokaryotes is most often carried out with vectors containing constitutive or inducible promoters directing the expression of either fusion or non-fusion polypeptides. Fusion vectors add a number of amino acids to a polypeptide encoded therein, usually to the amino terminus of the recombinant polypeptide but also to the C-terminus or fused within suitable regions in the polypeptides. Such fusion vectors typically serve three purposes: 1) to increase expression of a recombinant polypeptide; 2) to increase the solubility of a recombinant polypeptide; and 3) to aid in the purification of a recombinant polypeptide by acting as a ligand in affinity purification. Often, in fusion expression vectors, a proteolytic cleavage site is introduced at the junction of the fusion moiety and the recombinant polypeptide to enable separation of the recombinant polypeptide from the fusion moiety subsequent to purification of the fusion polypeptide. Such enzymes, and their cognate recognition sequences, include Factor Xa, thrombin, and enterokinase.

Typical fusion expression vectors include pGEX (Pharmacia Biotech Inc; Smith, D. B. and Johnson, K. S., 1988, Gene 67:31-40), pMAL (New England Biolabs, Beverly, Mass.), and pRIT5 (Pharmacia, Piscataway, N.J.) which fuse glutathione S-transferase (GST), maltose E binding polypeptide, or polypeptide A, respectively, to the target recombinant polypeptide. In one embodiment, the coding sequence of the CRIP1a or CRIP1b is cloned into a pGEX expression vector to create a vector encoding a fusion polypeptide comprising, from the N-terminus to the C-terminus, GST-thrombin cleavage site-X polypeptide. The fusion polypeptide can be purified by affinity chromatography using glutathione-agarose resin. Recombinant CRIP1a or CRIP1b unfused to GST can be recovered by cleavage of the fusion polypeptide with thrombin.

Examples of suitable inducible non-fusion E. coli expression vectors include pTrc (Amann et al., 1988, Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a co-expressed viral RNA polymerase (T7 gn1). This viral polymerase is supplied by host strains BL21(DE3) or HMS174(DE3) from a resident λ prophage harboring a T7 gn1 gene under the transcriptional control of the lacUV 5 promoter.

One strategy to maximize recombinant polypeptide expression is to express the polypeptide in a host bacteria with an impaired capacity to proteolytically cleave the recombinant polypeptide (Gottesman, S., Gene Expression Technology: Methods in Enzymology 185, Academic Press, San Diego, Calif. (1990) 119-128). Another strategy is to alter the sequence of the nucleic acid to be inserted into an expression vector so that the individual codons for each amino acid are those preferentially utilized in the bacterium chosen for expression, such as C. glutamicum (Wada et al., 1992, Nucleic Acids Res. 20:2111-2118). Such alteration of nucleic acid sequences of the invention can be carried out by standard DNA synthesis techniques.

In another embodiment, the CRIP1a or CRIP1b expression vector is a yeast expression vector. Examples of vectors for expression in yeast S. cerevisiae include pYepSec1 (Baldari, et al., 1987, EMBO J. 6:229-234), pMFa (Kujan and Herskowitz, 1982, Cell 30:933-943), pJRY88 (Schultz et al., 1987, Gene 54:113-123), and pYES2 (Invitrogen Corporation, San Diego, Calif.). Vectors and methods for the construction of vectors appropriate for use in other fungi, such as the filamentous fungi, include those detailed in: van den Hondel, C. A. M. J. J. & Punt, P. J., 1991, “Gene transfer systems and vector development for filamentous fungi,” in: Applied Molecular Genetics of Fungi, J. F. Peberdy, et al., eds., p. 1-28, Cambridge University Press: Cambridge.

Alternatively, the CRIP1a or CRIP1b polypeptides of the invention can be expressed in insect cells using baculovirus expression vectors. Baculovirus vectors available for expression of polypeptides in cultured insect cells (e.g., Sf 9 cells) include the pAc series (Smith et al., 1983, Mol. Cell Biol. 3:2156-2165) and the pVL series (Lucklow and Summers, 1989, Virology 170:31-39).

In yet another embodiment, a CRIP1a or CRIP1b nucleic acid of the invention is expressed in mammalian cells using a mammalian expression vector. Examples of mammalian expression vectors include pCDM8 (Seed, B., 1987, Nature 329:840) and pMT2PC (Kaufman et al., 1987, EMBO J. 6:187-195). When used in mammalian cells, the expression vector's control functions are often provided by viral regulatory elements. For example, commonly used promoters are derived from polyoma, Adenovirus 2, cytomegalovirus, and Simian Virus 40. For other suitable expression systems for both prokaryotic and eukaryotic cells, see chapters 16 and 17 of Sambrook, J., Fritsh, E. F., and Maniatis, T. Molecular Cloning: A Laboratory Manual. latest ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al., 1987, Genes Dev. 1:268-277), lymphoid-specific promoters (Calame and Eaton, 1988, Adv. Immunol. 43:235-275), in particular promoters of T cell receptors (Winoto and Baltimore, 1989, EMBO J. 8:729-733), and immunoglobulins (Baneji et al., 1983, Cell 33:729-740; Queen and Baltimore, 1983, Cell 33:741-748), neuron-specific promoters (e.g., the neurofilament promoter; Byrne and Ruddle, 1989, PNAS 86:5473-5477), pancreas-specific promoters (Edlund et al., 1985, Science 230:912-916), and mammary gland-specific promoters (e.g., milk whey promoter; U.S. Pat. No. 4,873,316 and European Application Publication No. 264,166). Developmentally-regulated promoters are also encompassed, for example, the murine hox promoters (Kessel and Gruss, 1990, Science 249:374-379) and the fetopolypeptide promoter (Campes and Tilghman, 1989, Genes Dev. 3:537-546).

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those that confer resistance to drugs, such as G418, hygromycin, and methotrexate. Nucleic acid molecules encoding a selectable marker can be introduced into a host cell on the same vector as that encoding a CRIP1a or CRIP1b or can be introduced on a separate vector. Cells stably transfected with the introduced nucleic acid molecule can be identified by, for example, antibiotic selection (e.g., cells that have incorporated the selectable marker gene will survive, while the other cells die).

According to the present invention, the introduced CRIP1a or CRIP1b may be maintained in the host cell stably if it is incorporated into a non-chromosomal autonomous replicon or integrated into a host cell chromosome. Alternatively, the introduced CRIP1b may be present on an extra-chromosomal non-replicating vector and may be transiently expressed or transiently active.

In one embodiment, a homologous recombinant microorganism can be created wherein the CRIP1a or CRIP1b is integrated into a chromosome, a vector is prepared which contains at least a portion of a CRIP1a or CRIP1b gene into which a deletion, addition, or substitution has been introduced to thereby alter, e.g., functionally disrupt, the CRIP1a or CRIP1b gene. Preferably, the CRIP1a or CRIP1b gene is a human or rat CRIP1b gene, but it can be a homolog from a related or unrelated organism. In one embodiment, the vector is designed such that, upon homologous recombination, the endogenous CRIP1a or CRIP1b gene is functionally disrupted (i.e., no longer encodes a functional polypeptide; also referred to as a knock-out vector). Alternatively, the vector can be designed such that, upon homologous recombination, the endogenous CRIP1a or CRIP1b gene is mutated or otherwise altered but still encodes a functional polypeptide (e.g., the upstream regulatory region can be altered to thereby alter the expression of the endogenous CRIP1a or CRIP1b). To create a point mutation via homologous recombination, DNA-RNA hybrids can be used in a technique known as chimeraplasty (Cole-Strauss et al., 1999, Nucleic Acids Research 27(5):1323-1330 and Kmiec, 1999, Gene Therapy American Scientist 87(3):240-247).

Whereas in the homologous recombination vector, the altered portion of the CRIP1a or CRIP1b gene is flanked at its 5′ and 3′ ends by an additional nucleic acid molecule of the CRIP1a or CRIP1b gene to allow for homologous recombination to occur between the exogenous CRIP1a or CRIP1b gene carried by the vector and an endogenous CRIP1a or CRIP1b gene. The additional flanking CRIP1a or CRIP1b nucleic acid molecule is of sufficient length for successful homologous recombination with the endogenous gene. Typically, several hundreds of base pairs up to kilobases of flanking DNA (both at the 5′ and 3′ ends) are included in the vector (See e.g., Thomas, K. R., and Capecchi, M. R., 1987, Cell 51:503 for a description of homologous recombination vectors).

In another embodiment, recombinant microorganisms can be produced that contain selected systems that allow for regulated expression of the introduced gene. Such regulatory systems are well known in the art.

Whether present in an extra-chromosomal non-replicating vector or a vector that is integrated into a chromosome, the CRIP1a or CRIP1b polynucleotide preferably resides in a mammalian expression cassette. A mammalian expression cassette preferably contains regulatory sequences capable of driving gene expression in mammalian cells that are operatively linked so that each sequence can fulfill its function, for example, termination of transcription by polyadenylation signals.

Gene expression should be operatively linked to an appropriate promoter conferring gene expression in a timely, cell specific, or tissue specific manner. Promoters useful in the expression cassettes of the invention include any promoter that is capable of initiating transcription in a host cell. The promoter may be constitutive, inducible, developmental stage-preferred, cell type-preferred, tissue-preferred, or organ-preferred.

The invention further provides a recombinant expression vector comprising a CRIP1a or CRIP1b DNA molecule of the invention cloned into the expression vector in an antisense orientation. That is, the DNA molecule is operatively linked to a regulatory sequence in a manner that allows for expression (by transcription of the DNA molecule) of an RNA molecule that is antisense to a CRIP1a or CRIP1b mRNA. Regulatory sequences operatively linked to a nucleic acid molecule cloned in the antisense orientation can be chosen which direct the continuous expression of the antisense RNA molecule in a variety of cell types. For instance, viral promoters and/or enhancers, or regulatory sequences can be chosen which direct constitutive, tissue specific, or cell type specific expression of antisense RNA. The antisense expression vector can be in the form of a recombinant plasmid, phagemid, or attenuated virus wherein antisense nucleic acids are produced under the control of a high efficiency regulatory region. The activity of the regulatory region can be determined by the cell type into which the vector is introduced. For a discussion of the regulation of gene expression using antisense genes, see Weintraub, H. et al., 1986, Antisense RNA as a molecular tool for genetic analysis, Reviews-Trends in Genetics, Vol. 1(1), and Mol et al., 1990, FEBS Letters 268:427-430.

Another aspect of the invention pertains to host cells into which a recombinant expression vector of the invention has been introduced. The terms “host cell” and “recombinant host cell” are used interchangeably herein. It is understood that such terms refer not only to the particular subject cell but they also apply to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic or eukaryotic cell. For example, a CRIP1a or CRIP1b can be expressed in bacterial cells such as C. glutamicum, insect cells, fungal cells, or mammalian cells (such as Chinese hamster ovary cells (CHO) or COS cells), algae, ciliates, plant cells, fungi, or other microorganisms like C. glutamicum. Other suitable host cells are known to those skilled in the art.

A host cell of the invention, such as a prokaryotic or eukaryotic host cell in culture, can be used to produce (i.e., express) a CRIP1a or CRIP1b. Accordingly, the invention further provides methods for producing CRIP1a or CRIP1b polypeptides using the host cells of the invention. In one embodiment, the method comprises culturing the host cell of the invention (into which a recombinant expression vector encoding a CRIP1a or CRIP1b has been introduced, or into which genome has been introduced a gene encoding a wild-type or altered CRIP1a or CRIP1b) in a suitable medium until the CRIP1a or CRIP1b is produced. In another embodiment, the method further comprises isolating CRIP1a or CRIP1b polypeptides from the medium or the host cell.

Another aspect of the invention pertains to isolated CRIP1a or CRIP1b polypeptides, and biologically active portions thereof. An “isolated” or “purified” polypeptide or biologically active portion thereof is free of some of the cellular material when produced by recombinant DNA techniques, or chemical precursors or other chemicals when chemically synthesized. The language “substantially free of cellular material” includes preparations of CRIP1a or CRIP1b in which the polypeptide is separated from some of the cellular components of the cells in which it is naturally or recombinantly produced. In one embodiment, the language “substantially free of cellular material” includes preparations of a CRIP1a or CRIP1b having less than about 30% (by dry weight) of non-CRIP1a or non-CRIP1b material (also referred to herein as a “contaminating polypeptide”), more preferably less than about 20% of non-CRIP1a or non-CRIP1b material, still more preferably less than about 10% of non-CRIP1a or non-CRIP1b material, and most preferably less than about 5% non-CRIP1a or non-CRIP1b material.

When the CRIP1a or CRIP1b or biologically active portion thereof is recombinantly produced, it is also preferably substantially free of culture medium, i.e., culture medium represents less than about 20%, more preferably less than about 10%, and most preferably less than about 5% of the volume of the polypeptide preparation. The language “substantially free of chemical precursors or other chemicals” includes preparations of CRIP1a or CRIP1b in which the polypeptide is separated from chemical precursors or other chemicals that are involved in the synthesis of the polypeptide. In one embodiment, the language “substantially free of chemical precursors or other chemicals”includes preparations of a CRIP1a or CRIP1b having less than about 30% (by dry weight) of chemical precursors or non-CRIP1a or non-CRIP1b chemicals, more preferably less than about 20% chemical precursors or non-CRIP1a or non-CRIP1b chemicals, still more preferably less than about 10% chemical precursors or non-CRIP1a or non-CRIP1b chemicals, and most preferably less than about 5% chemical precursors or non-CRIP1a or non-CRIP1b chemicals. In preferred embodiments, isolated polypeptides, or biologically active portions thereof, lack contaminating polypeptides from the same organism from which the CRIP1a or CRIP1b is derived. Typically, such polypeptides are produced by recombinant expression of, for example, a human or rat CRIP1a or CRIP1b in organisms other than human or rat, or microorganisms such as C. glutamicum, ciliates, algae, or fungi.

The nucleic acid molecules, polypeptides, polypeptide homologs, fusion polypeptides, primers, vectors, and host cells described herein can be used in one or more of the following methods: evolutionary studies; determination of CRIP1a or CRIP1b regions required for function; modulation of CRIP1a or CRIP1b activity; and modulation CB1 receptor activity.

In a preferred embodiment, transcription of the CRIP1a or CRIP1b is modulated using zinc-finger derived transcription factors (ZFPs) as described in Greisman and Pabo, 1997, Science 275:657 and manufactured by Sangamo Biosciences, Inc. These ZFPs comprise both a DNA recognition domain and a functional domain that causes activation or repression of a target nucleic acid such as a CRIP1a or CRIP1b nucleic acid. Therefore, activating and repressing ZFPs can be created that specifically recognize the CRIP1a or CRIP1b promoters described above and used to increase or decrease CRIP1a or CRIP1b expression in a plant, thereby modulating the activity of CB1. The present invention also includes identification of the homologs of CRIP1a or CRIP1b as defined in SEQ ID NO:2, SEQ ID NO:5, and SEQ ID NO:7 in a target cell, as well as the homolog's promoter. The invention also provides a method of increasing expression of a gene of interest within a host cell as compared to a wild type variety of the host cell, wherein the gene of interest is transcribed in response to CRIP1a or CRIP1b expression, comprising: (a) transforming the host cell with an expression vector comprising a CRIP1a or CRIP1b coding nucleic acid, and (b) expressing the CRIP1a or CRIP1b within the host cell, thereby increasing the expression of the gene transcribed in response to the expression of CRIP1a or CRIP1b, as compared to a wild type variety of the host cell.

The CRIP1a or CRIP1b nucleic acid molecules of the invention are also useful for evolutionary and polypeptide structural studies. By comparing the sequences of the nucleic acid molecules of the present invention to those encoding similar polypeptides from other organisms, the evolutionary relatedness of the organisms can be assessed. Similarly, such a comparison permits an assessment of which regions of the sequence are conserved and which are not, which may aid in determining those regions of the polypeptide that are essential for the functioning of the polypeptide. This type of determination is of value for polypeptide engineering studies and may give an indication of what the polypeptide can tolerate in terms of mutagenesis without losing function.

Manipulation of the CRIP1a or CRIP1b nucleic acid molecules of the invention may result in the production of CRIP1a or CRIP1b polypeptides having functional differences from the wild-type CRIP1a or CRIP1b polypeptides. These polypeptides may be improved in efficiency or activity, may be present in greater numbers in the cell than is usual, or may be decreased in efficiency or activity.

Additionally, the sequences disclosed herein, or fragments thereof, can be used to generate knockout mutations in the genomes of various organisms. The resultant knockout cells can then be evaluated for the effect on CB1 activity using various assays. For other methods of gene inactivation, see U.S. Pat. No. 6,004,804 “Non-Chimeric Mutational Vectors” and Puttaraju et al., 1999, Spliceosome-mediated RNA trans-splicing as a tool for gene therapy, Nature Biotechnology 17:246-252. The aforementioned mutagenesis strategies for CRIP1a or CRIP1b polypeptides resulting in increased or decreased CB1 activity are not meant to be limiting; variations on these strategies will be readily apparent to one skilled in the art.

The present invention also provides antibodies that specifically bind to a CRIP1a or CRIP1b polypeptide, or a portion thereof, as encoded by a nucleic acid described herein. Antibodies can be made by many well-known methods (See, e.g., Harlow and Lane, “Antibodies; A Laboratory Manual,” Cold Spring Harbor Laboratory, Cold Spring Harbor, N.Y., (1988)). Briefly, purified antigen can be injected into an animal in an amount and in intervals sufficient to elicit an immune response. Antibodies can either be purified directly, or spleen cells can be obtained from the animal. The cells can then fused with an immortal cell line and screened for antibody secretion. The antibodies can be used to screen nucleic acid clone libraries for cells secreting the antigen. Those positive clones can then be sequenced. (See, for example, Kelly et al., 1992, Bio/Technology 10:163-167; Bebbington et al., 1992, Bio/Technology 10:169-175).

The phrases “selectively binds” and “specifically binds” with the polypeptide refer to a binding reaction that is determinative of the presence of the polypeptide in a heterogeneous population of polypeptides and other biologics. Thus, under designated immunoassay conditions, the specified antibodies bound to a particular polypeptide do not bind in a significant amount to other polypeptides present in the sample. Selective binding of an antibody under such conditions may require an antibody that is selected for its specificity for a particular polypeptide. A variety of immunoassay formats may be used to select antibodies that selectively bind with a particular polypeptide. For example, solid-phase ELISA immunoassays are routinely used to select antibodies selectively immunoreactive with a polypeptide. See Harlow and Lane, “Antibodies, A Laboratory Manual” Cold Spring Harbor Publications, New York, (1988), for a description of immunoassay formats and conditions that could be used to determine selective binding.

In some instances, it is desirable to prepare monoclonal antibodies from various hosts. A description of techniques for preparing such monoclonal antibodies may be found in Stites et al., eds., “Basic and Clinical Immunology,” (Lange Medical Publications, Los Altos, Calif., Fourth Edition) and references cited therein, and in Harlow and Lane “Antibodies, A Laboratory Manual” Cold Spring Harbor Publications, New York, 1988.

The present invention provides methods of modulating the activity of a CB1 cannabinoid receptor, comprising administering an effective amount of a composition to the CB1 cannabinoid receptor. In one embodiment, the composition comprises a polypeptide encoded by a polynucleotide selected from the group consisting of a polynucleotide as defined in SEQ ID NO:1, SEQ ID NO:3, SEQ ID NO:4, or SEQ ID NO:6, a polynucleotide encoding a polypeptide as defined in SEQ ID NO:2, SEQ ID NO:5, or SEQ ID NO:7 and a polynucleotide complementary to a full-length polynucleotide thereof. In another embodiment, the composition comprises an antibody that selectively binds a polypeptide encoded by a polynucleotide selected from the group consisting of a polynucleotide as defined in SEQ ID NO:1, SEQ ID NO:3, SEQ ID NO:4, or SEQ ID NO:6, a polynucleotide encoding a polypeptide as defined in SEQ ID NO:2, SEQ ID NO:5, or SEQ ID NO:7 and a polynucleotide complementary to a full-length polynucleotide thereof. In another embodiment, the composition comprises a nucleic acid selected from the group consisting of a polynucleotide as defined in SEQ ID NO:1, SEQ ID NO:3, SEQ ID NO:4, or SEQ ID NO:6, a polynucleotide encoding a polypeptide as defined in SEQ ID NO:2, SEQ ID NO:5, or SEQ ID NO:7 and a polynucleotide complementary to a full-length polynucleotide thereof.

In particular, the methods of the present invention for modulating the activity of the CB1 cannabinoid receptor in a patient by administering a CRIP1a or CRIP1b composition may result in one or more effects selected from the group consisting of appetite stimulation, analgesia, euphoria, decreased tremor or spasticity associated with multiple sclerosis, attenuation of nausea and vomiting in cancer chemotherapy, reduction of intraocular pressure, decreased intestinal motility, and attenuation of aversive memories. The present invention also provides methods for modulating the activity of the CB1 cannabinoid receptor in a patient by administering a CRIP1a or CRIP1b composition which may result in one or more effects selected from the group consisting of appetite suppression, decreased sedation, decreased alterations in cognition and memory, and decreased mood alterations. In one embodiment, the methods of the present invention are used to modulate the activity of CB1 in glutamatergic neurons. In another embodiment, the methods for modulating the activity of CB1 are used for a patient that is suffering from epilepsy. In yet another embodiment, the methods for modulating the activity of CB1 are used for a patient that is suffering from drug or alcohol addiction or post traumatic stress disorder.

As used herein, the term “glutamatergic neuron” refers to an excitatory neuron, and the term “GABAergic neuron” refers to an inhibitory neuron. Glutamatergic neurons play a role in neural development, synaptic plasticity, learning and memory, epilepsy, neural ischemia, drug addiction, tolerance, neuropathic pain, anxiety, and depression, among other conditions. Glutamate is the main excitatory neurotransmitter in the mammalian brain and is known to participate in higher order processes, such as development, learning, and memory. As an excitatory amino acid (EAA), glutamate also plays a role in neuropathologic events, including cell death, that result from excessive stimulation of post-synaptic neurons (i.e., excitotoxic damage). Glutamate binds or interacts with one or more glutamate receptors (GluRs), which can be divided into two different classes, the ionotropic receptors (iGluR) and the metabotropic receptors (mGluR).

The iGluRs are ligand-gated ion channels that, upon binding glutamate, open to allow the selective influx of certain monovalent and divalent cations, thereby depolarizing the cell membrane. In addition, certain iGluRs with relatively high calcium permeability can activate a variety of calcium-dependent intracellular processes. In contrast to the iGluRs, the metabotropic glutamate receptors (mGluRs) are G protein-coupled receptors capable of activating a variety of intracellular second messenger systems following the binding of glutamate. Activation of mGluRs in mammalian neurons can decrease the activity of ion channels, including ligand-gated channels such as iGluRs. Several subtypes of mGluRs have been isolated by molecular cloning. In addition, the various subtypes of mGluRs have been divided into three groups based on amino acid sequence homologies, the second messenger systems they utilize, and pharmacological characteristics (Nakanishi, 1994, Neuron 13:1031).

During a period of anoxia (e.g., cardiopulmonary resuscitation), ischemic stroke, epileptic seizure, and other types of CNS injury, certain iGluRs are overactivated (Choi, 1992, Neurobiol 9:1261-96; Zipfel et al., 2000 J Neurotrauma 10:857-69; Fountain, 2000, Epilesia 41 Suppl 2:S23-30; Tanaka et al., 2000, Brain Res 886(1-2):190-207; Pujol et al., 1999, Ann NY Acad Sci 884:249-254). The net result of this effect is a massive increase in the concentration of intracellular calcium, triggering a series of events leading to neuronal death (Sapolsky, 2001, J Neurochem 76(6):1601-1611). Functional overactivity of iGluRs has also been implicated in a variety of neurodegenerative diseases, such as lateral sclerosis, Alzheimer's disease, Huntington's chorea, and AIDS dementia syndrome (Tanaka H et al., 2000, Brain Res 886(1-2):190-207).

The compositions of this invention further comprise a pharmaceutically acceptable carrier. The phrases “pharmaceutically or pharmacologically acceptable” refer to molecular entities and compositions that do not produce an adverse, allergic, or other untoward reaction when administered to an animal, or a human, as appropriate. Veterinary uses are equally included within the invention and “pharmaceutically acceptable” formulations include formulations for both clinical and/or veterinary use. As used herein, “pharmaceutically acceptable carrier” includes any and all solvents, dispersion media, coatings, antibacterial, and antifungal agents, isotonic and absorption delaying agents, and the like. The use of such media and agents for pharmaceutically active substances is well known in the art. Except insofar as any conventional media or agent is incompatible with the active ingredient, its use in the therapeutic compositions is contemplated. For human administration, preparations should meet sterility, pyrogenicity, and general safety and purity standards as required by FDA Office of Biologics standards. Supplementary active ingredients can also be incorporated into the compositions.

As used herein with respect to these methods, the term “administering” refers to various means of introducing a composition into a cell or into a patient. These means are well known in the art and may include, for example, injection; tablets, pills, capsules, or other solids for oral administration; nasal solutions or sprays; aerosols, inhalants; topical formulations; liposomal forms; and the like. As used herein, the term “effective amount” refers to an amount that will result in the desired result and may readily be determined by one of ordinary skill in the art.

The CRIP1a or CRIP1b compositions of the present invention may be formulated for parenteral administration, e.g., formulated for injection via the intravenous, intramuscular, sub-cutaneous, transdermal, or other such routes. The preparation of an aqueous composition that contains such a protein, antibody, or nucleic acid as an active ingredient will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form should be sterile and fluid to the extent that syringability exists. It should be stable under the conditions of manufacture and storage and should be preserved against the contaminating action of microorganisms, such as bacteria and fungi.

The CRIP1a or CRIP1b compositions can be formulated into a sterile aqueous composition in a neutral or salt form. Solutions as free base or pharmacologically acceptable salts can be prepared in water suitably mixed with a surfactant, such as hydroxypropylcellulose. Pharmaceutically acceptable salts, include the acid addition salts (formed with the free amino groups of the protein), and those that are formed with inorganic acids such as, for example, hydrochloric or phosphoric acids, or such organic acids as acetic, trifluoroacetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine, and the like.

Suitable carriers include solvents and dispersion media containing, for example, water, ethanol, polyol (for example, glycerol, propylene glycol, and liquid polyethylene glycol, and the like), suitable mixtures thereof, and vegetable oils. In many cases, it will be preferable to include isotonic agents, for example, sugars, or sodium chloride. The proper fluidity can be maintained, for example, by the use of a coating, such as lecithin, by the maintenance of the required particle size in the case of dispersion and/or by the use of surfactants.

Under ordinary conditions of storage and use, all such preparations should contain a preservative to prevent the growth of microorganisms. The prevention of the action of microorganisms can be brought about by various antibacterial and antifungal agents, for example, parabens, chlorobutanol, phenol, sorbic acid, thimerosal, and the like. Prolonged absorption of the injectable compositions can be brought about by the use in the compositions of agents delaying absorption, for example, aluminum monostearate, and gelatin.

Prior to or upon formulation, the CRIP1a or CRIP1b compositions should be extensively dialyzed to remove undesired small molecular weight molecules, and/or lyophilized for more ready formulation into a desired vehicle, where appropriate. Sterile injectable solutions are prepared by incorporating the active agents in the required amount in the appropriate solvent with various of the other ingredients enumerated above, as desired, followed by filter sterilization. Generally, dispersions are prepared by incorporating the various sterilized active ingredients into a sterile vehicle that contains the basic dispersion medium and the required other ingredients from those enumerated above.

In the case of sterile powders for the preparation of sterile injectable solutions, the preferred methods of preparation are vacuum-drying and freeze-drying techniques that yield a powder of the active ingredient, plus any additional desired ingredient from a previously sterile-filtered solution thereof.

Suitable pharmaceutical compositions in accordance with the invention will generally include an amount of the CRIP1a or CRIP1b, anti-CRIP1a or anti-CRIP1b antibody, or CRIP1a or CRIP1b nucleic acid admixed with an acceptable pharmaceutical diluent or excipient, such as a sterile aqueous solution, to give a range of final concentrations, depending on the intended use. The techniques of preparation are generally well known in the art as exemplified by Remington's Pharmaceutical Sciences, 16th Ed. Mack Publishing Company, 1980, incorporated herein by reference. It should be appreciated that for human administration, preparations should meet sterility, pyrogenicity, and general safety and purity standards as required by FDA Office of Biological Standards.

The present invention also provides methods of screening for a modulator of CRIP1a or CRIP1b activity on a CB1 cannabinoid receptor, comprising combining a CB1 cannabinoid receptor and a CRIP1a or CRIP1b polypeptide in the presence and absence of a putative modulator, and comparing the interaction between the CB1 cannabinoid receptor and the CRIP1a or CRIP1b polypeptide in the presence and absence of the putative modulator, wherein the CRIP1a or CRIP1b polypeptide comprises a polypeptide as defined in SEQ ID NO:2, SEQ ID NO:5, or SEQ ID NO:7.

Throughout this application, various publications are referenced. The disclosures of all of these publications and those references cited within those publications in their entireties are hereby incorporated by reference into this application in order to more fully describe the state of the art to which this invention pertains.

It should also be understood that the foregoing relates to preferred embodiments of the present invention and that numerous changes may be made therein without departing from the scope of the invention. The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention and/or the scope of the appended claims.

### EXAMPLES

### Example 1

Identification of proteins That Interact with CB1 Cannabinoid Receptor

A human brain cDNA library (Clontech) containing 5×10⁶ independent clones with an average insert size of 1.5 kb fused to the GALA activation domain was cotransformed into Y 190 yeast cells with a bait plasmid consisting of the amino acids at positions 418-472 of the human cannabinoid receptor (CB1) fused to the GALA DNA binding domain. An overnight culture of Y190 cells was diluted to an OD₆₀₀ of 0.08 (20 ml/reaction) and incubated at 30° C. until an OD₆₀₀ of 0.2-0.4. The cells were washed in 10 μl of water and resuspended in 200 ml of 100 mM LiAc, 10 mM Tris-HCl, pH 7.5, and 1 mM EDTA (LiAc/TE). Single stranded herring testis DNA (200 μg), 20 μg of bait plasmid, 20 μg of carrier plasmid, and 1.2 ml of 40% polyethyleneglycol/LiAc/TE were added to the cells, followed by incubation at 30° C. for 30 minutes at 225 rpm. DMSO was added at a tenth of the final volume and cells were heat-shocked at 42° C. for 15 minutes. Then, YPD media (2% peptone, 1% yeast extract, 2% glucose, pH 5.8) were added, and the cells were incubated at 30° C. at 250 rpm for 2 hours. Cells were resuspended in TE buffer, and 1/200 volume was plated on −Leu −Trp plates to determine transformation efficiency. The rest was plated on −Leu −Trp −His plates containing 2.5 mM 3-aminotriazole. After incubation of the plates at 30° C. for 3-5 days, β-galactosidase activity of the colonies was assessed by lifting colonies onto filters, lysing the colonies by freezing in liquid nitrogen, and exposing the filters to 20 mg/ml X-gal in phospate-containing buffer. Seventeen colonies that exhibited β-galactosidase activity were chosen for further analysis. Only clones that showed a specific interaction with the C-terminal region of CB1 were chosen for further analysis.

To isolate plasmid DNA from His+Lac+ colonies, 2 ml of yeast cultures were grown for 2-3 days in selective media, and cells were lysed in 2% TritonX-100, 1% sodium dodecyl sulfate (SDS), 100 mM NaCl, 10 mM Tris-HCl pH. 8.0, and 1 mM EDTA along with added phenol-chloroform-isoamyl alcohol (25:24:1) and vortexed in the presence of acid-washed glass beads. The DNA in the aqueous phase was precipitated with ethanol and resuspended in TE buffer. The DNA was used to transform JM109 E. coli cells, which were plated for selection on ampicillin plates. Bacterial plasmid DNA was prepared by standard protocols (Sambrook et al., 1989, Molecular Cloning: A Laboratory Manual, Cold Spring Harbor, N.Y.).

### Example 2

Molecular Characterization of CRIP1a and CRIP1b

The 5′ sequence of the clone was obtained using the 5′-RACE technique. Primers were designed based on the sequence identified as being the insert in the yeast two-hybrid vector. The SMART™ RACE cDNA Amplification Kit (Clontech, Calif.) and human brain cDNA library with end-adapters was used. The resulting products were cloned into pBlueScript for sequencing. One clone was designated CB1 Receptor Interacting Protein 1b (CRIP1b), and its nucleic acid sequence and deduced amino acid sequence are shown in SEQ ID NO:1 and SEQ ID NO:2, respectively. The nucleic acid encodes a 128 amino acid polypeptide encoded by three exons and is located on human chromosome 2. The human CRIP1b clone isolated from the yeast two hybrid library had 563 nucleotides including a 3′ untranslated region of 275 nucleotides with a poly(A) tail consisting of 18 adenine nucleotides. CRIP1b has no transmembrane domains but does have consensus sites for protein kinase C phosphorylation at amino acids 37-39 and 47-49 and casein kinase II phosphorylation at amino acids 108-111 (which represents the junction between exons 2 and 3).

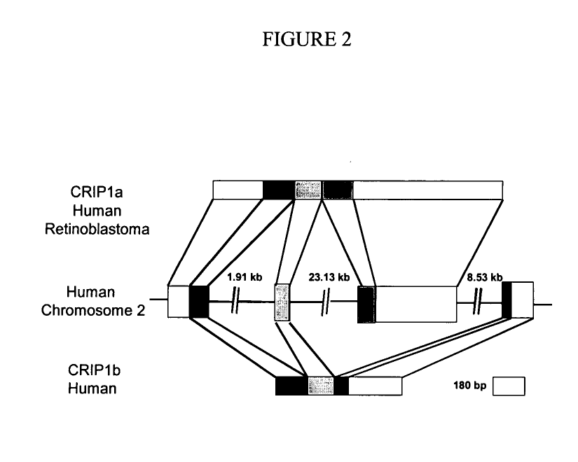

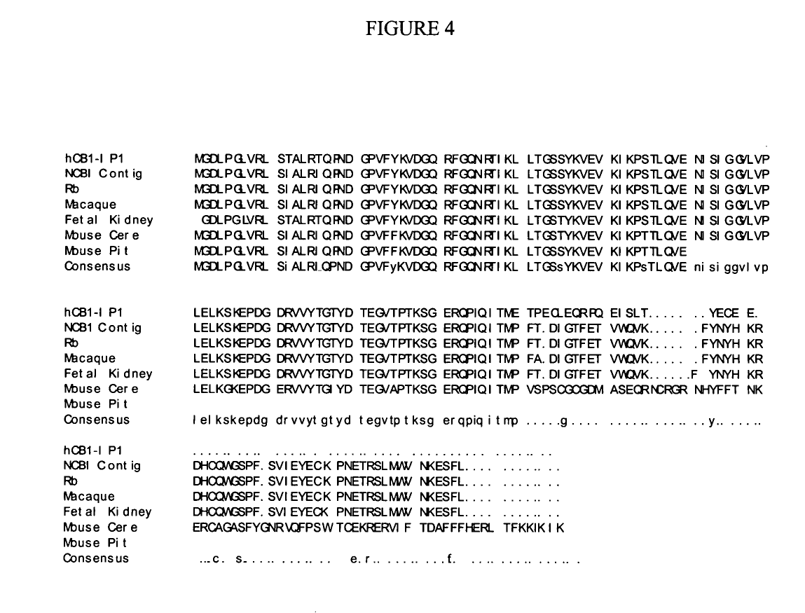

Human CRIP1b showed homology to clones in the NCBI database derived from human fetal kidney, human retinoblastoma, and macaque brain, among other sequences (FIG. 4). Analysis of human genome sequence data indicates that the 128 amino acid polypeptide (CRIP1b) and the human retinoblastoma polypeptide are encoded by the same gene which comprises four exons (1, 2, 3a, and 3b) that is alternatively spliced to generate different cDNAs (FIG. 2). CRIP1a comprises exons 1, 2, and 3a, whereas CRIP1b comprises exons 1, 2, and 3b (FIG. 2). Exon-intron analysis reveals a 1.91 kb intron following exon 1 and a 23.13 kb intron following exon 2 for the human CRIP1a or retinoblastoma clones. In CRIP1b, the third exon follows an additional 8.53 kb intron.

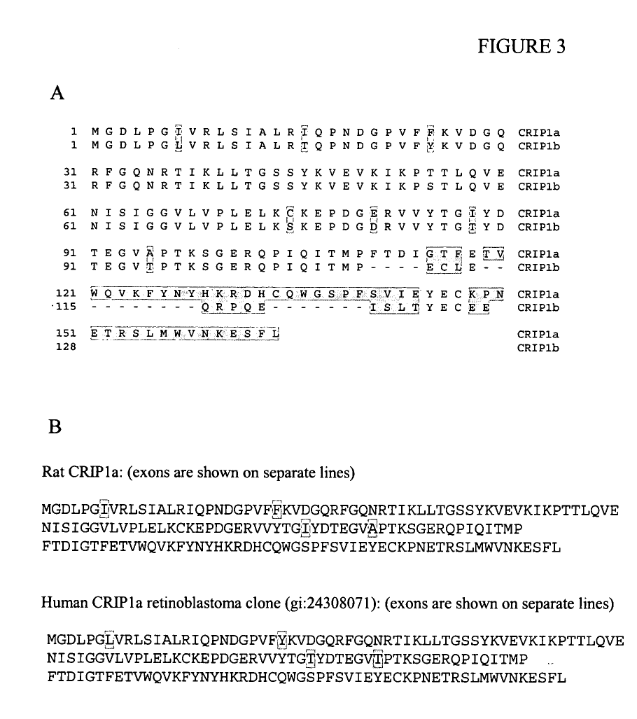

Using PCR primers to the human CRIP1b sequence to probe rat brain cDNA, a protein was cloned and named rat CRIP1a. The complete CRIP1a nucleic acid sequence from rat was determined. The nucleic acid sequence and deduced amino acid sequence for rat CRIP1a are shown in SEQ ID NO:6 and SEQ ID NO:7, respectively. The rat brain cDNA encodes a related polypeptide of 164 amino acids. Exons 1 and 2 of the rat CRIP1a and the human CRIP1b have the same number of amino acids, with only 7 amino acid differences between the two sequences (FIG. 3A). Human CRIP1b exon 3 has 18 amino acids, and rat CRIP1a exon 3 has 54 amino acids. The human retinoblastoma gene appears to be the human ortholog of the CRIP1a cloned from rat brain. Rat CRIP1a is 96% identical to the human retinoblastoma clone (gi:24308071) located on chromosome 2 (FIG. 3B); therefore the human sequence was designated human CRIP1a. There are only 4 amino acid differences between rat CRIP1a and the human CRIP1a sequences, and these differences are in exons 1 and 2. The amino acids of exon 3 are identical between rat CRIP1a and the human CRIP1a sequences. The nucleic acid sequence and deduced amino acid sequence for human CRIP1a are shown in SEQ ID NO:4 and SEQ ID NO:5, respectively.

The CRIP1b amino acid sequence also was used to blast the GenBank public database for similar sequences. An alignment of a portion of the results of this search is shown in FIG. 4. A comparative analysis of the chimpanzee, mouse, and rat genomes revealed that while exons 1, 2, and 3a occur in all three species, exon 3b is found only in chimpanzee and human. Based on these findings, the 164 amino acid polypeptide is referred to as CRIP1a, and the 128 amino acid polypeptide is referred to as CRIP1b (formerly known as CB1-IP1). Orthologues of CRIP1a are also present in chick and puffer fish, and therefore, CRIP1a likely is present throughout vertebrates, whereas CRIP1b is unique to a subset of mammals including humans and chimpanzees. A search of the genomes of Drosophila and Ciona as representatives of protostomian and deuterostomian invertebrates, respectively, yielded no orthologs of either CRIP1b or CRIP1a. These data suggest that the CRIP1a cannabinoid receptor interacting protein may have first evolved in a primitive vertebrate and that CRIP1b may be unique to primates or even humans.

### Example 3

Characterization of Interaction Between CRIP1b and CB1 Receptor

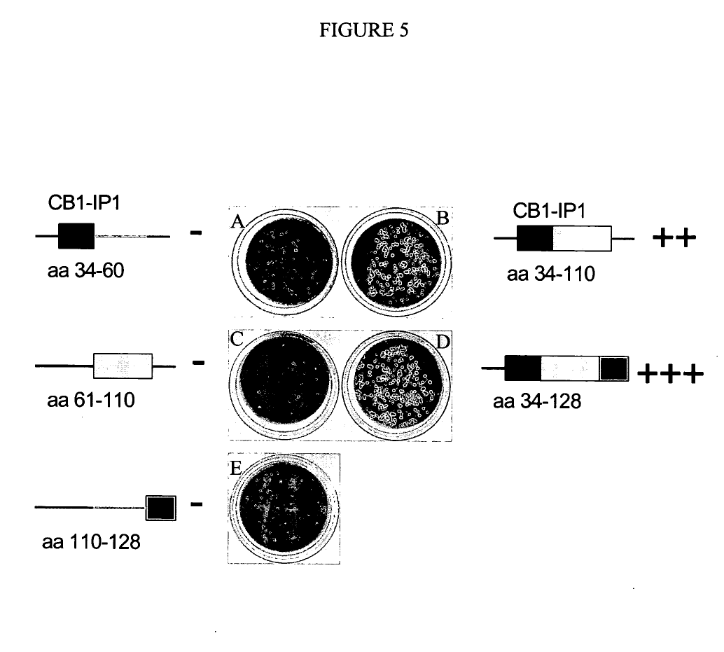

To determine which domains of CRIP1b interact with the C-terminus (amino acids 418-472) of the CB1 cannabinoid receptor, yeast two hybrid experiments were performed. CRIP1b was divided into three parts, including part of exon 1 (amino acids 34-60 of SEQ ID NO:2), exon 2 (amino acids 61-110 of SEQ ID NO:2) and exon 3 (amino acids 111-128 of SEQ ID NO:2). Each part was tested for interaction alone and in combination. When each exon was tested alone, no interaction was detected (FIGS. 5A, 5C, and 5E). Exon 1 (amino acids 34-60 of SEQ ID NO:2) and exon 2 (amino acids 61-110 of SEQ ID NO:2) in combination showed a positive interaction that was enhanced with the addition of exon 3 (amino acids 111-128 of SEQ ID NO:2) (FIGS. 5B and 5D). Because CRIP1a is 96% identical at the amino acid level to exons 1 and 2 of CRIP1b, it is anticipated that these domains of CRIP1a will interact with the CB1 cannabinoid receptor.

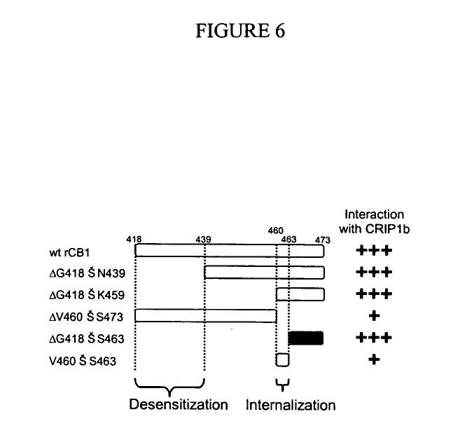

Results of yeast two-hybrid experiments also demonstrated that CRIP1b interacts with the C-terminal region of the C-terminal tail of CB1 (amino acids 464-473 of rat CB1) (FIG. 6). The yeast two-hybrid technique was used to determine the CB1 receptor interaction site with CRIP1b. The C-terminus of the CB1 receptor contains separate domains critical for desensitization and internalization (Hsieh et al., 1999; Jin et al., 1999). The results of the yeast two hybrid experiments demonstrated that neither of these domains interacted strongly with CRIP1b. The most distal 9 amino acids of the CB1 receptor was the minimal domain tested that gave a strong interaction with CRIP1b (FIG. 6). Interestingly, the last 9 amino acids are well conserved between mammals and the puffer fish Fugu, suggesting that the acquisition of the distal C-terminal tail region of the CB1 cannabinoid receptor may have been the key factor for co-evolution of the CB1 receptor interacting proteins.

### Example 5

Affinity Chromatography of CRIP1b and CB1 Cannabinoid Receptor

To confirm the interaction between the CB1 cannabinoid receptor and the interacting protein CRIP1b, in vitro pull-down assays of bacterially expressed proteins were performed. A GST fusion peptide was created between GST and a peptide of the CB1 receptor by cloning the polynucleotide sequence encoding amino acids at position 418-472 of the CB1 receptor into the pGEX-4T-1 expression vector. This GST fusion peptide was expressed and purified using standard protocols. GST alone and purified GST/CB1 fusion peptide were coupled to a first and second Glutathione Sepharose-4B column, respectively, as per standard protocols (Smith and Johnson, 1988, Gene, 67:31-40).

Polymerase chain reaction (PCR) was used to amplify the amino acids at positions 34-128 of CRIP1b in the pCT2 shuttle vector with EcoRI and XhoI restriction endonuclease sites. The PCR product was inserted in-frame into a pET30C expression plasmid harboring an S-tag (Novagen, Wis.). The resulting plasmid was transformed into BL21(DE3) E. coli cells, and these cells were induced to express CRIP1b by the introduction of isopropyl β-D-thiogalactopyranoside.

The bacteria were lysed in a buffer containing 50 mM Tris-Cl (pH 8.0), 1 mM EDTA, 100 mM NaCl, phenylmethylsulfonylfluoride (PMSF) (8 μl of 50 mM PMSF per gram of bacterial pellet). The mixture was stirred at 37° C.; DNAse was added (20 μl of 1 mg/ml solution per gram of bacterial pellet); and the mixture was incubated at room temperature until it became clear. The lysate was centrifuged twice at 12,000×g for 15 minutes to recover the protein in the supernatant. Proteins present as inclusion bodies were solubilized using standard protocols (Sambrook et al., 1989, Molecular Cloning: A Laboratory Manual, Cold Spring Harbor, N.Y.).

The lysate was loaded first onto the GST column, and the unbound protein suspension from this column was loaded on to the GST fusion peptide column. The second column was washed, and the proteins were eluted with reduced glutathione. Eluted fractions from the second column were separated by SDS polyacrylamide gel electrophoresis (SDS-PAGE) (Laemmli, 1970, Nature, 227:680-685). Proteins were Coomassie blue stained or electrotransferred to a nitrocellulose membrane (Towbin et al., 1979, Proc. Natl. Acad. Sci. USA, 76:4350). The membrane was incubated with an appropriate S protein conjugate prior to detection with chemiluminescent substrate (Novagen, Wis.).

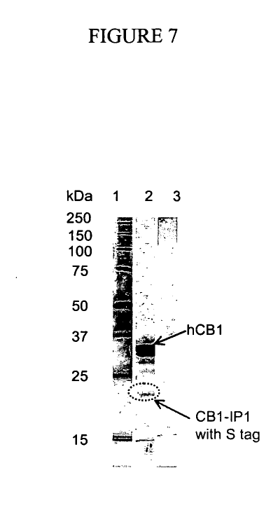

FIG. 7 shows that CRIP1b (identified as CB1-IP1) and the CB1 receptor C-terminal region (amino acids 418-472) eluted from the glutathione-Sepharose 4B matrix (lane 2). CRIP1b from bacterial lysate did not bind to glutathione Sepharose-4B on its own (lane 3).

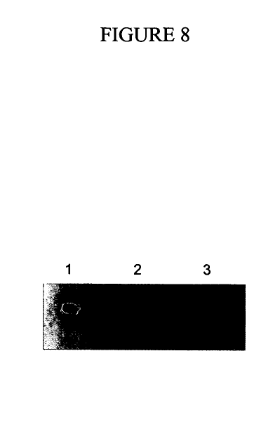

The identity of CRIP1b was further confirmed by probing a Western membrane containing the eluates with an S-tag probe (FIG. 8). FIG. 8, lane 1 shows original bacterial lysate of CRIP1b; lane 2 depicts the eluate from a CRIP1b lysate added to CB1(418-472)-containing glutathione-Sepharose 4B matrix; and lane 3 depicts the eluate from a CRIP1b lysate added to matrix without the CB1 cannabinoid receptor fragment attached (again, there is no CRIP1b detected). In summary, GST-CB1 (418-472) pulled down CRIP1b, showing that these two proteins interact in vitro. The identity of S-tagged CRIP1b was verified with the S-tag probe. In control experiments, CRIP1b did not bind to the column in the absence of GST-CB1 (418-472) fusion proteins.

The results of these experiments further indicated that the amino acids at positions 34-128 of CRIP1b are capable of interaction with the amino acids at positions 418-472 of the CB1 receptor.

### Example 6

Production and Use of Polyclonal Antibodies to CRIP1b

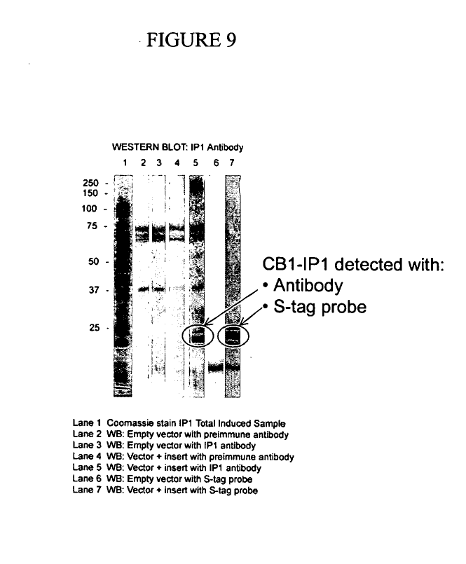

Chicken polyclonal antibodies specific to CRIP1b were developed by a Strategic Solutions (Newark, Del.). A unique fourteen amino acid peptide in the C-terminal region of the human CRIP1a or CRIP1b polypeptide was conjugated to keyhole limpet hemocyanin. This conjugate was injected into two chickens, and an IgY population was affinity-purified from egg yolks and examined for antibody titer using ELISA. Control IgY antibody was obtained from the egg yolks prior to injection of the chickens with the conjugate. Additionally, preimmune and immune sera were collected from these chickens. The specificity of the immune sera was determined by Western blot (FIG. 9). Lane 1 contained Coomassie blue stain of the CRIP1b protein induced in bacteria; lane 2 is a Western blot of a control consisting of an empty vector probed with preimmune serum; lane 3 is a Western blot of a control consisting of an empty vector probed with CRIP1b antibody; lane 4 is a Western blot of a control consisting of CRIP1b probed with preimmune serum; lane 5 is a Western blot of CRIP1b probed with CRIP1b antibody; lane 6 is a Western blot using the S-tag probe that detects the S-tag protein from the empty vector; and lane 7 is a Western blot using the S-tag probe to verify the identity of the CRIP1b protein. The CRIP1b antibody recognized CRIP1b, and the identity of the CRIP1b protein was verified using the S-tag probe (FIG. 9).

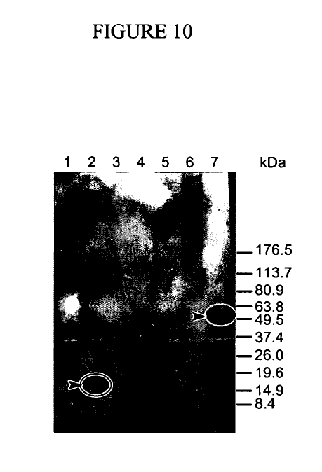

Proteins from various human tissues were obtained from Biochain (Hayward, Calif.). These proteins were separated on an SDS-PAGE gel, and a western blot of the gel was prepared as per standard protocols. Digoxigenin-labeled anti-CRIP1b antibody was used to probe the Western blot containing proteins from various human tissues, and the distribution and molecular weight of the reactive proteins was analyzed. These experiments demonstrated that an approximately 52 kDa CRIP1b protein is present in brain tissue and that an approximately 15 kDa CRIP1b protein is present in heart tissue (FIG. 10). The predicted molecular weight of the CRIP1b protein is 13 kDa, suggesting that CRIP1b may be present in the brain as a tetramer.

### Example 7

Determination of the Tissue Distribution of CRIP1a Expression

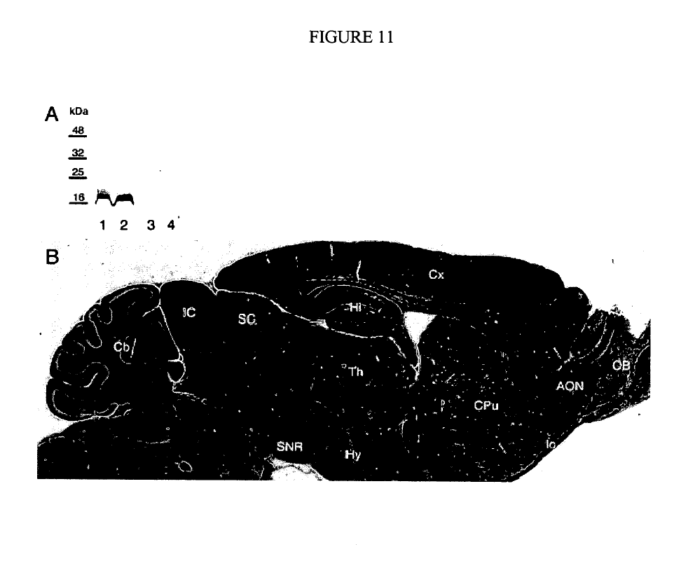

Specific antibodies to CRIP1a were developed, following immunization of rabbits using a peptide comprising the C-terminal 17 amino-acids of the mouse CRIP1a protein sequence. Serum was collected following two booster immunizations. Analysis of sera using dot-blot assays and ELISAs revealed the presence of anti-peptide antibodies (data not shown). Western blot analysis of mouse brain homogenates using antisera (1:1000 dilution) revealed specific staining of a single band with a molecular mass consistent with that expected for mouse CRIP1a (18.5 kDa). No cross-reacting bands were evident (FIG. 11A).

Using the CRIP1a antibody for detection, CRIP1a protein was found to be present in extracts of mouse brain, AtT-20 cells, N18TG2 cells and cultured rat cerebellar granule neurons (CGN), but not HEK293 cells as shown by Western blot. Western blot analysis of homogenates of a variety of mouse organs/tissues indicated that CRIP1a is also expressed in other organs apart from the brain. An 18 kDa band that reacts with CRIP1a antibodies can be detected in lung, heart, spleen, liver, kidney, skeletal muscle, testis and intestine. However, the concentration of CRIP1a in the brain is much higher than any other organ analyzed. This is an important funding because it suggests that the primary role of CRIP1a may be for regulation of neural signaling mechanisms. The intensity of the 18 kDa band is high in lung, heart, and intestine, with intermediate intensity in kidney and testis, and low intensity in liver, spleen, and skeletal muscle.

Immunocytochemical analysis of mouse brain sections using the CRIP1a antisera (1:1000) revealed widespread staining in many regions of the brain (FIG. 11B), and importantly, all of the staining observed is blocked by pre-absorption of antisera with antigen peptide. Also, the intensity of the staining is quite high, consistent with the detection by Western blot. Examples of neurons were identified in which CRIP1a and CB1 are co-expressed and co-distributed (e.g. cerebellar granule cells). Therefore, it is clear that there is potential for interaction between CRIP1a and CB1 in vivo.

Analysis of the pattern of immunostaining in the brain indicates that CRIP1a is largely associated with neurons, with little or no staining evident in any glial cells. From a functional perspective, it is important to determine precisely which neuronal types express CRIP1a in different regions of the brain. Nevertheless, the preliminary analysis of immunostained mouse brain sections indicates that CRIP1a is expressed in, for example, hippocampal CA3 pyramidal cells, neocortical pyramidal cells (notably in layers 5/6), olfactory bulb mitral cells, and cerebellar granule cells. Of particularly interest are neocortical layer 5/6 pyramidal cells because these cells project to subcortical regions of the brain, including the striatum and thalamus. Widespread CRIP1a-IR (CRIP1a immunoreactivity) is present in the striatum and thalamus but CRIP1a-IR is not evident in neuronal somata in these brain regions. Therefore, it is suspected that the CRIP1a-IR in the striatum and thalamus may be associated with the axons of layer 516 cortical pyramidal cells. Consistent with the intense CRIP1a-IR in the cell bodies of hippocampal CA3 pyramidal cells, intense CRIP1a-IR is also evident in the stratum radiatum (Rad) of the CA1 region where the axons of CA3 neurons terminate (data not shown). In the cerebellum, the most intense CRIP1a-IR is located in parallel fibers in the molecular layer, which is consistent with CRIP1a expression by cerebellar granule cells. These data provide the basis for some general observations about CRIP1a expression in the rodent brain: a) in neurons that express CRIP1a, the protein is located in somatodendritic and axonal compartments, and b) neurons that express CRIP1a are principal neurons in several major regions of the brain (neocortex, hippocampus, olfactory bulb), which project within or between brain regions. These two observations, when combined, likely explain why CRIP1a-IR occurs extensively in many brain regions.

Like CRIP1a, CB1 is widely expressed in many regions of the brain (Egertová and Elphick, 2000). Comparative analysis of the distribution of CB1 and CRIP1a reveals, that while CRIP1a and CB1 are co-localized in some populations, there are also neurons that express CB1 but not CRIP1a and neurons that express CRIP1a but not CB1.

An example of neurons where CB1 and CRIP1a are co-expressed and co-localized in axons are cerebellar granule cells (data not shown). Another example, where co-localization of CB1 and CRIP1a is evident is in the striatum. Here, it is likely that CB1and CRIP1a are co-localized in the axons of cortico-striatal neurons. In the hippocampas, there is evidence of CB1 mRNA expression in CA3 pyramidal cells (Matsuda et al., 1990), but detailed analysis of glutamatergic axon terminals in the hippocampus has not revealed the presence of CB1 (Katona et al., 1999). Therefore, co-localization of CRIP1a and CB1 in axons of hippocantpal pyramidal cells, although theoretically possible, may not occur in vivo.

An example of a neuronal population where CB1 is expressed without CRIP1a is the striatal GABAergic neurons that project to the globus pallidus, entopeduncular nucleus, and the substantia nigra. Thus, the substantia nigra pars reticulata contains little or no CRIP1a-IR (FIG. 11E), but has very intense CB1-IR (Egertová and Elphick, 2000; Egertová et al., 2003).

The majority of CB1 immunoreactivity in cortical regions of the brain (e.g. neocortex, hippocampus) occurs in local GABAergic interneurons, where it is targeted to axon terminals surrounding the somata and/or dendrites of principal cells (data not shown). Based on the patterns of CRIP1a-IR that we have observed in the mouse brain, it appears that CRIP1a and CB1 are not co-localized in these neurons. An area of the brain where it is easy to observe GABAergic axon terminals is the cerebellar cortex and here CB1-IR is localized in basket cell terminals surrounding Purkinje cells (Egertová and Elphick, 2000; Egertová et al., 2003). Interestingly, CRIP1a-IR is not evident in basket cell terminals (data not shown). Therefore, absence of CRIP1a expression may be a general feature of CB1-expressing GABAergic neurons in the brain.

An example of neurons where CRIP1a is expressed without CB1 are mitral cells in the olfactory bulb-intense CRIP1a-IR is present in the somata of mitral cells but little or no CB1 mRNA expression is detected in mitral cells (Matsuda et al., 1990). These data indicate that CRIP1a may interact with other proteins in addition to CB1.

The preliminary analysis of CRIP1a expression in the rodent brain and comparison with the distribution of CB1 has provided an important basis of investigation of CRIP1a function in the brain. Firstly, it is clear that there are neurons in the brain where CB1 and CRIP1a are co-expressed and colocalized, and where CRIP1a could therefore interact with CB1 to modulate its activity (e.g. cerebellar granule cells and neocortical pyramidal cells). What these neurons have in common is that they are glutamatergic excitatory neurons, whereas CB1-expressing GABAergic inhibitory neurons appear to lack expression of CRIP1 a (see above). Therefore, it is speculated that co-expression of CRIP1a may be a special feature of CB1-expressing glutamatergic excitatory neurons.

### Example 8

Expression of CRIP1b in Various Cell Lines and Primary Neuronal Cultures

To identify the pattern of expression of CRIP1a and CRIP1b, various cell lines and primary neuronal cultures were analyzed. RT-PCR was performed using standard protocols to detect the expression of CRIP1a and CRIP1b in each of the cell lines and cultures, and the samples were subjected to electrophoreses. Cells or brain tissue was homogenized in Ultraspec RNA solution (Biotecx). The lysate was incubated, chloroform was added, and the sample was vortexed for 15 seconds and then incubated on ice for 5 minutes. The lysate was then centrifuged for 15 minutes at 4° C. and 12,000×g. The upper phase was removed, and 1 μl 20 mg/ml glycogen was added together with an equal volume of isopropanol. The lysate was mixed and incubated at 4° C. for 10 minutes. The RNA was pelleted at 12,000×g for 10 minutes at 4° C. The pellet was washed once with 75% ethanol, centrifuged for 5 minutes at 4° C. at 12,000×g, air dried, and resuspended in DEPC-treated water. The RNA was then preprecipitated, pelleted, washed with 75% ethanol and resuspended in DEPC-treated water. The sample was treated with RQ1 DNase (Promega) and divided into 2 aliquots. Reverse transcription with random primers (Retroscript kit from Ambion) was performed on 1 sample, and the other sample was used as a “no reverse transcriptase” control. Then 1-2 μl of each transcription reaction was used for PCR amplification using gene specific primers. PCR products were visualized on 2% agarose gels in TBE buffer containing ethidum bromide.

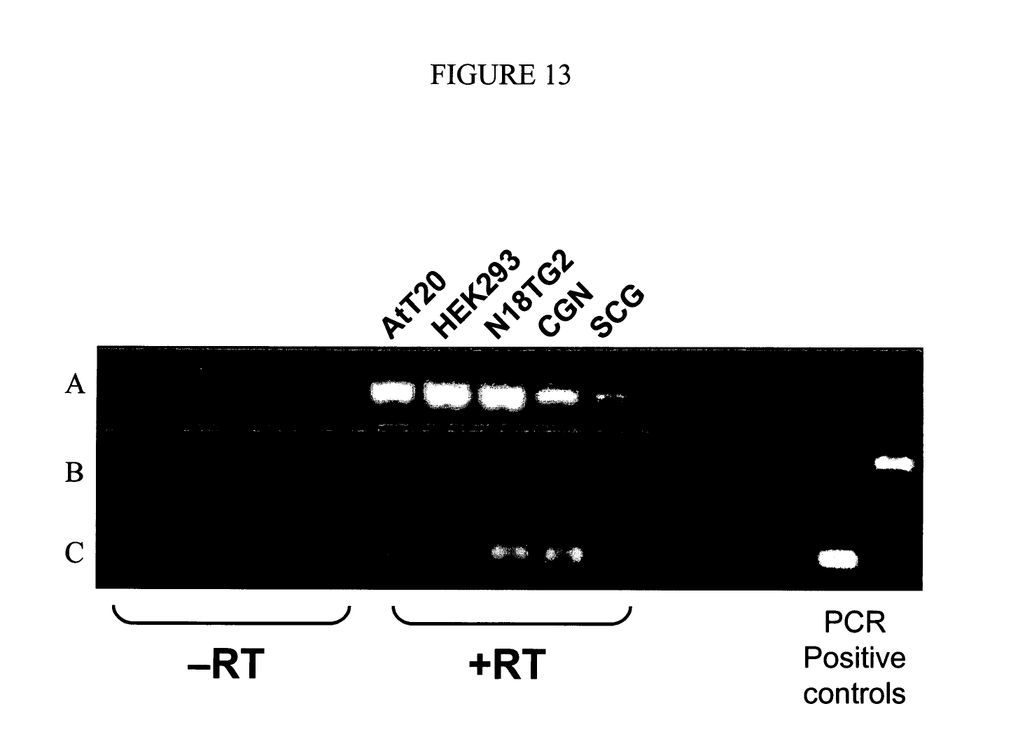

The results of these experiments show that CRIP1a, but not CRIP1b, was detected in HEK 293, AtT20, N18TG2, rat superior cervical ganglion, and rat cerebellar granule neurons (FIG. 13).

### Example 9

Functional Characterization of CRIP1a

Various experiments are known by one of ordinary skill in the art that may be used to determine the level of rat CB1 activity. For example, rat CRIP1a is subcloned into mammalian expression vector pCI or pcDNA3. Rat superior cervical ganglion (SCG) neurons were dissociated with 0.3 mg/ml trypsin, 0.45 mg/ml collegenase D (Boehringer), and 0.1 mg/ml DNase in Earle's Balanced Salt solution and plated onto poly-L-lysine-coated culture dishes in Minimum Essential Medium with 10% FBS, 1% glutamine and 1% penicillin-streptomycin. The nuclei of the SCG neurons are microinjected with rCB1 and rat CRIP1a (100 ng/μl) together with the co-injection marker pEGFP-N1 (10 ng/μl) using an Eppendorf 5246 microinjector and 5171 micromanipulator system.

Ca2⁺ currents were recorded using the whole-cell patch clamp technique (Hamill et al., 1981, Pfluegers Arch., 391:85-100). Ca2⁺ currents were elicited by voltage steps from a holding potential of −80 mV and digitized at 180 μsec per point. A double pulse protocol consisting of two 25 ms steps to +5 mV was used to elicit Ca2⁺ currents. Statistical significance was determined by Student's test, p<0.05.

To isolate Ca2⁺ currents, cells are bathed in an external solution containing: 140 mM tetraethylammonium (TEA) methanesulfonate, 10 mM HEPES, 15 mM glucose, 10 mM CaCl₂, 0.0001 mM tetrodoxin, pH 7.4. The intracellular solution contains: 120 mM N-methyl-D-glucamine, 20 mM TEA chloride, 10 mM HEPES, 11 mM EGTA, 1 mM CaCl2, 4 mM MgATP, 0.1 mM Na2GTP, 14 mM phosphocreatine, pH 7.2. A fast switching device was used to apply the control external solution or the external solution containing one of the drug solutions. Stock solutions of 10 mM WIN 55,212-2 mesylate (the CB1 agonist, RBI/Sigma), SR141716A (the CB1 inverse agonist, NIDA), and UK14304 (α₂-adrenergic agonist) were prepared in DMSO. WIN 55,212-2 and SR 141716A were diluted into the external solution and briefly sonicated to facilitate dispersion. The final concentration of DMSO is <0.01% which has had no effect on the Ca2⁺ current in previous experiments. Stock solutions of 10 mM norepinephrine and 1 mM (D-Trp⁸)-somatostatin-14 were made in water. All stock solutions were stored at −20° C. Neurons were treated overnight with pertussis toxin or cholera toxin at 500 ng/ml.

The CB1 receptor inverse agonist SR141716 increases the voltage-gated Ca²⁺ current in SCG neurons expressing CB1 receptors (Pan et al., 1998). The enhancement of the Ca²⁺ current in the presence of SR141716 is due to the ability of SR141716 to bind to the inactive state of the CB1 receptor with higher affinity (Hurst et al., 2002). Thus, the effect of SR141716 is to reverse the constitutive activity of CB1 receptors, resulting in an increase in the voltage-gated Ca²⁺ current (Pan et al., 1998; Lewis, 2004).

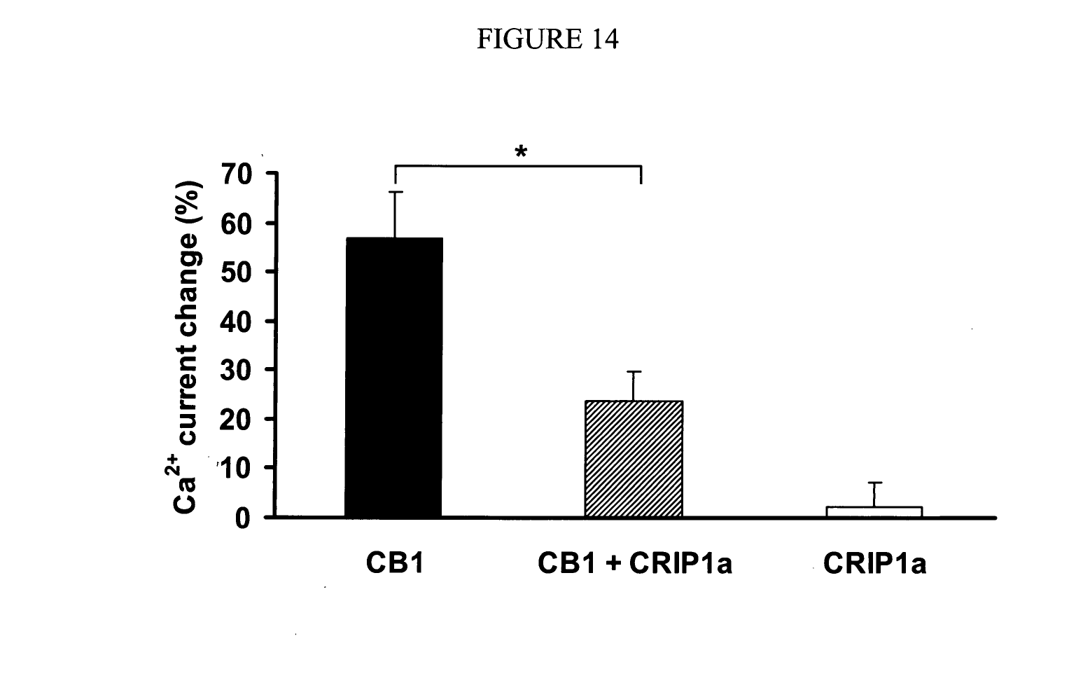

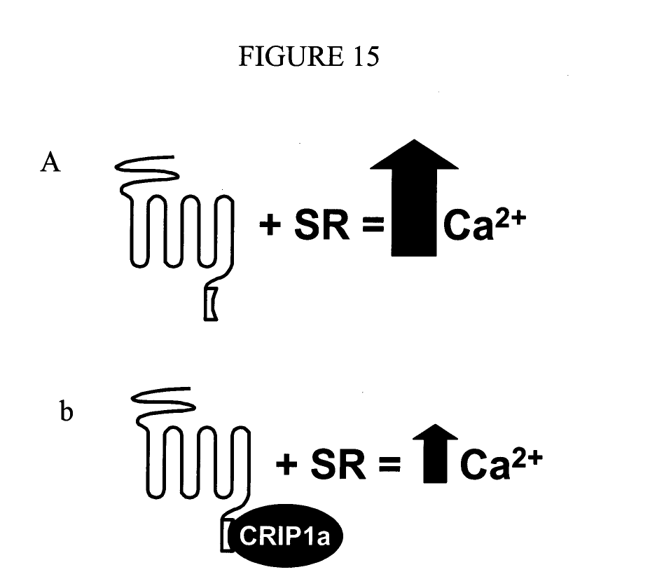

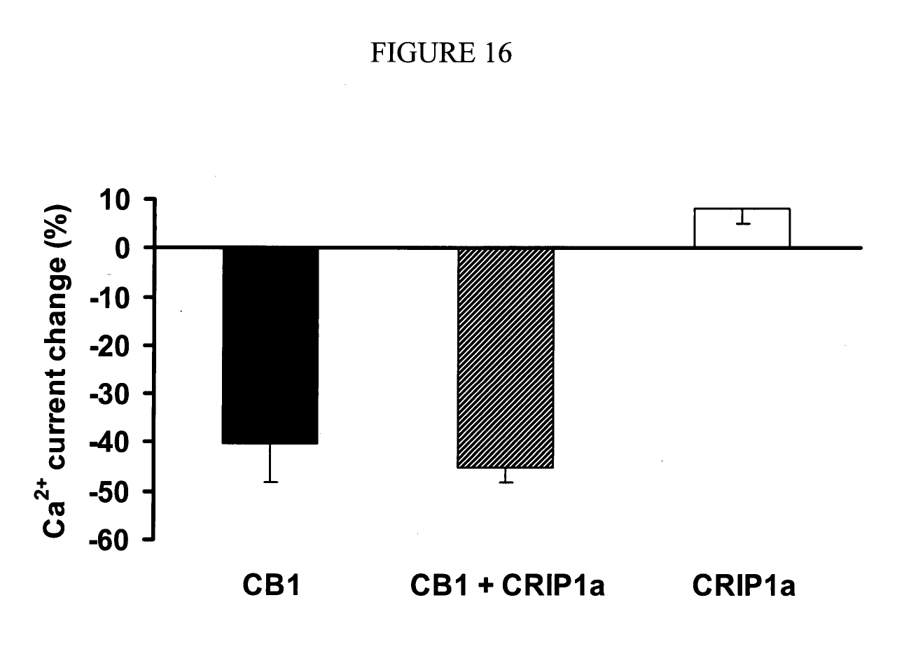

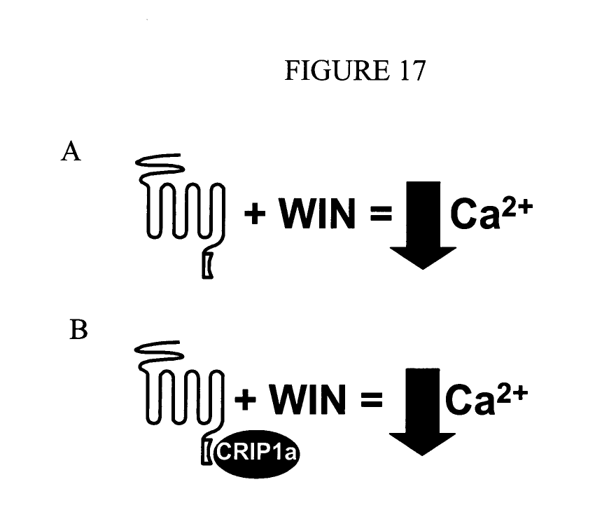

Co-expression of CRIP1a with CB1 receptors significantly inhibited the effect of SR141716 while expression of CRIP1a alone had no effect (FIGS. 14 and 15). These results are consistent with the interpretation that CRIP1a decreases the constitutive activity of CB1 receptors. An alternative explanation is that CRIP1a decreases CB1 expression thereby reducing the number of receptors in the active state available for SR141716 to bind. However, CRIP1a had no effect on the agonist-induced inhibition of the Ca²⁺ current in SCG neurons expressing both CB1 and CRIP1a (FIGS. 16 and 17), suggesting that CRIP1a does not change receptor expression or affect the agonist response. Thus, CRIP1a appears to preferentially inhibit the constitutive activity of wild type CB1 receptors.

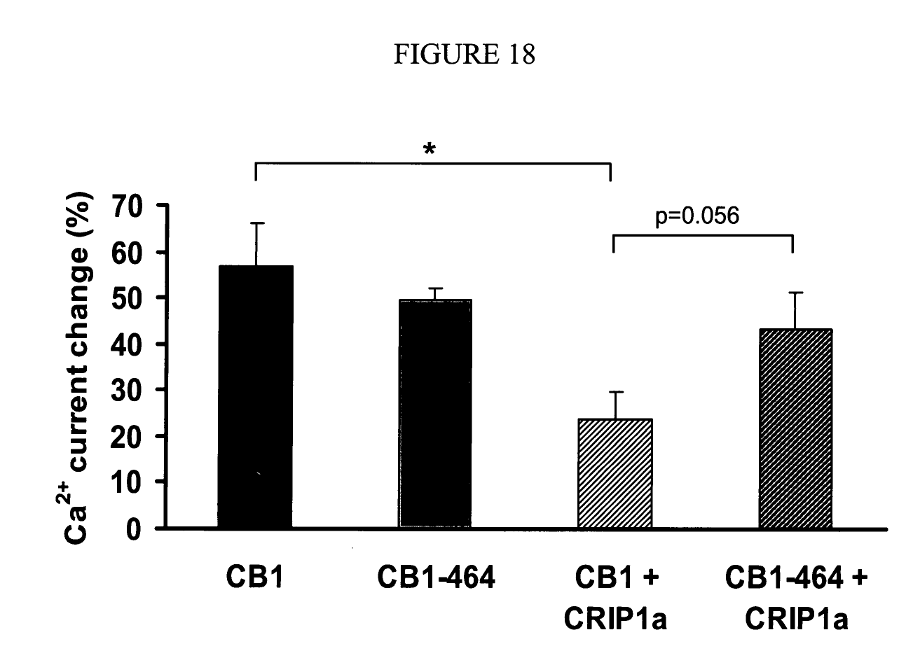

Deletion of the distal 9 amino acids of the CB1 receptor (CB1-464) tended to abolish the effect of CRIP1a and restore the ability of SR141716 to increase the Ca²⁺ current (FIG. 18). SR141716 induced Ca²⁺ current increase was not significantly altered in the truncated receptor compared to the wild type receptor. These results suggest that CRIP1a decreases the constitutive activity of CB1 receptors and that this effect may be by interacting with the distal 9 amino acids of the CB1 receptor. Another measure of constitutive activity is the Ca²⁺ current facilitation ratio. Facilitation is thought to be a reversal of Ca²⁺ current inhibition by G protein coupled receptors (Bean, 1989; Ikeda, 1991; Ehrlich and Elmslie, 1995). A larger facilitation ratio indicates greater constitutive activity of G protein-coupled receptors. The basal facilitation ratio in SCG neurons is 1.17±0.01 without CB1 receptors (Pan et al., 1998). The facilitation ratio is 1.46±0.08 in SCG neurons expressing CB1 receptors, but is decreased to 1.26±0.03 when co-expressed with CRIP1a. These results demonstrate that the CB1 receptor is constitutively active and that CRIP1a decreases the constitutive activity of CB1. The facilitation ratio data also demonstrates that CRIP1a does not decrease the ability of SR141716 to bind CB1 receptors, but instead prevents the CB1 receptor from adopting an active conformational state.

Deletion of the distal 9 amino acids of CB1 (CB1-464) surprisingly enhanced agonist-induced inhibition of the Ca²⁺ current in the presence of CRIP1a compared to expression of the wild type receptor with CRIP1a. However, the effect of the agonist WIN55,212-2 on the truncated receptor (CB1-464) when compared to the wild type CB1 receptor showed a trend to increase that was close to significance. This result may indicate that CRIP1a binds to a site on the CB1 receptor other than the distal 9 amino acids of the C-terminus to enhance the effect of the agonist WIN55,212-2 or that deletion of the distal 9 amino acids itself enhances the effect of WIN55212-2.

In another experiment, cerebellar granule neurons were used. Cerebellar granule neurons express endogenous CB1 cannabinoid receptors. The cerebellum of postnatal day 5-8 rats was enzymatically digested in 0.3 mg/ml trypsin and 0.1 mg/ml DNase in Earle's balanced salt solution for 30 minutes at 35° C. Neurons were counted using a hemocytometer, plated onto poly-L-lysine coated 35 mm culture dishes at 500,000 cells/dish and incubated at 37° C. in 5% CO₂. After 72 hours, cerebellar granule cells were transfected using polyethyleneimine (0.1 M, 25 kDa) in a ratio of 3.25 μg DNA/μl PEI.

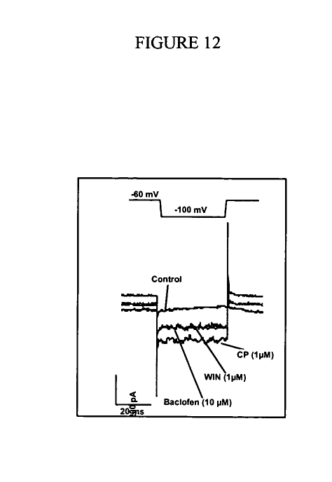

Inwardly rectifying K⁺ (GIRK) currents in cerebellar granule neurons were recorded using the patch clamp technique in an external solution containing: 120 mM NaCl, 30 mM KCl, 10 mM HEPES, 15 mM glucose, 1.5 mM CaCl2, and 2.5 mM MgCl at pH 7.2. The intracellular solution contains: 140 mM potassium gluconate, 10 mM HEPES, 5 mM KCl, 0.2 mM EGTA, 3 mM MgATP, and 0.3 mM Na₂GTP at pH 7.2. The cerebellar granule neurons were clamped to a holding potential of −60 mV in solutions containing 30 mM KCl and designed to isolate G protein-coupled inwardly rectifying K⁺ (GIRK) currents. The membrane potential was stepped to −100 mV in the absence and presence of the cannabinoid agonists WIN 55,212-2 (1 μM) and CP 55,940 (1 μM) or the GABA_{B} receptor agonist baclofen (10 μM) (FIG. 12). GIRK currents were activated by the cannabinoid agonists WIN 55,212-2 and CP 55,940 and by the GABA_{B} receptor agonist baclofen (FIG. 12). These results indicate that granule neurons express both cannabinoid and GABA_{B} receptors that activate GIRK currents. Granule neurons may be used to test the effects of CB1 cannabinoid receptor interacting proteins, CRIPs, on activation of GIRK current by CB1 cannabinoid receptors. GABA_{B} receptor activation of GIRK current may be used as a control.

### APPENDIX

cDNA Sequence of CRIP1b from Homo sapiens (SEQ ID NO:1)

GGCCAGAGCGAGCCTTCGGGTGCGTGGAGGGCGCAGCACTGGAGCGGGGG
ATGGGGAAGCGCGCGGCCTTGGAGCCGTCTCTTGGGCCCGCTTCCTCGCG
GAGTGGGGAGGGGGGCCACAGCCTAAGGAGCTGCTCCTCAAAAGCCCGGG
CGGACGGCGGCGACGGCGCGGGTTCCTAGCGGCAGCCTCAGCTCCACCAC
CTCCCTCACCCTCCCCGCGCTTCCTCTCCGCCCCTCGCGCTCCAGCCACT
CGGCCGCAGCCGGCGCTGTCCTCCGCCCCCCGGAGCCGCCGCGCCAGACC
CTCGCCCAGACATGGGGGACCTGCCGGGCCTCGTGCGCCTCTCCATCGCG
CTGCGCATCCAGCCTAATGACGGCCCGGTCTTTTACAAGGTGGACGGGCA
GCGCTTCGGCCAGAACCGCACCATCAAGCTGCTCACCGGCTCCTCCTACA
AGGTTGAGGTGAAGATTAAGCCCAGCACGCTGCAGGTCGAGAATATTTCC
ATTGGTGGTGTGCTTGTCCCACTGGAACTGAAGTCTAAAGAGCCTGATGG
GGACAGAGTTGTTTATACGGGTACATATGACACAGAAGGTGTGACCCCAA
CGAAGAGTGGAGAACGGCAACCCATCCAGATCACCATGCCGGAGTGCCTG
GAACAACGACCCCAAGAGATCTCTCTTACATATGAATGTGAAGAATAAGG
ACATTCTTCAATGTGTGCCATCATGAGGAACAAATGTTCTTTTTTTTTTT
TCCAAATTATTTGCTTTGCATTCTAGGGGAGGGCTTCTCACAGGCCATCA
GAATTATCGGGGATGCTTGTTAAAATGTAGATTCCTAAACTCTACCTTTT
TAGACCTCTTAAATTGAGCAAACTCTCTGAGAGTAAATCCAAAGCATCTG
CATTTTTATTAAGCACTTTAGGTGATTCTTATCATATTAAAGTTTGGGAA
AACACTAAAAAAAAAAAAAAAAAA

Deduced amino acid sequence of CRIP1b from Homo sapiens (SEQ ID NO:2)

MGDLPGLVRLSIALRTQPNDGPVFYKVDGQRFGQNRTIKLLTGSSYKVEV
KIKPSTLQVENISIGGVLVPLELKSKEPDGDRVVYTGTYDTEGVTPTKSG
ERQPIQITMPECLEQRPQEISLTYECEE

Genomic sequence of CRIP1b from Homo sapiens (SEQ ID NO:3)

AGTGTTTTCCAAACTTTAATATGATAAGAATCACCTAAAGTGCTTAATAA
AATGCAGATGCTTTGGATTTACTCTCAGAGAGTTTGCTCAATTTAAGAGG
TCTAAAAAGGTAGAGTTTAGGAATCTACATTTTAACAAGCATCCCCGATA
ATTCTGATGGCCTGTGAGAAGCCCTCCCCTAGAATGCAAAGCAAATAATT
TGGAAAAAAAAAAAAGAACATTTGTTCCTCATGATGGCACACATTGAAGA
ATGTGCTTATTCTTCACATTCATATGTAAGAGAGATCTCTTGGGGTCGTT
GTTCCAGGCACTCCTGAAAATAAATAGATACCAGAATAAGTTTGAGAAAA
CCACAGTTGGGTAGGTGCAGTGACTCACGCCCGTAATCCCAGCACTTTGG
GAGGCCAAGGCAGGCAGATCTCTTGAGGTCAGGAGTTCGAGACCAGCTGA
CCAATATGGTGAAGCCCCATCTCTACTAAAAATACAAAAATTAGCTGGCT
GTGGTAGTGGTGCACCTGTAGTCCCAGCTACTTGGGAGGCTGAGGCAGAG
AATCACTTGAAGCTGGGAGGCAGAGGTTGCAGTGAGCTGAGATTGTGCCA
CTGCACTCCAGCCTGAGTGACAGAGCGAGACTCTGTCTCAAAAAAAAAAA
AAAAAAAGAAAAGAAAACCACAGCGAAGCGAATTCTGTGAAGACTATAAA
GAAGCATCAAGCTCTGTGTAAAGGGCTAGATTGTTTCTTTGTAAGGACAA
TTGTCATCAAATCCCATATGTTTGTTTTGTTAAAATAGCCACTATTTAGC
CAGGCCCAAGCTGTTTTCACTAATACACTGCATTTCTGTCAAGCTTCTCT
TTGGATGTTCTCCAAGCATTTTAGCAGCTTTAAGCCTGTCTTCCCTTGTT
CCCTGAGGGAGGAACAAGCACAAGTTCACGCCAACTCATTAGAGAGACAT
TGCTCAAGAGCAGCTCCTGGGGCACCAGATGCAGGCAGGAGCGAGGGAGT
GGGATCCCTTCCAGGCCCCTCCCCTCTGCGCTGTCTCATTTTCCCCACCC
ACCCTCCCAAATGCTCATTATTGTCTTCAGCAGGTGGCTTCCACCCCTGC
CTTCAAGTGTGCTCAGACAATCTCCCTCCCAAGATAGACAAGCTCCAAAA
AGATTCACAGGTTCTACACAATGCAGAACTGCCAAGGACCACATTGGCCA
AAGCAATGTCTTCATACAATCAATCTTAAGGGATTTTGCAGAGACAATGG
TAAATGTGGATTAGGCTTTTGATCAGAAGGCAGAATATTCGTGACCAGCC
TGGGCAACATGGTGAAACCCATCTCTACAAAAATTAGCTGGATGTGGTGG
TGGCCGCCTGTGGTCCCAAATACTTGGGAGGCTGAGGTGAGAGGATCACT
TGAGACTGGGAGGTTGAGGCTGCAGTGAGCCAACATCGCACCACTGCACT
CCAGCATGGGCTGTTACAGAGCAAGACCCTGTCTCAAAAAAAAAAAAAAA
AGAAAAGAAAAGAAAAGAAAAGAAAGAAAGGCAGAACCTTTAGGCAGCAG
ATGTCCACCAGAGGTATGTGTGCATGTGTGTGCATGTGCACGTGTGTGTA
CAGCCATATGGGTTTTAATACATGTATAGATTCATTCAACCACTGCCGCT
ATCAGGATACAGAACAGTTCCATCGCCGCAGAAAGCTCCCTCCTGCTACC
TTTTTGAGTGTATCCAAGCTTTATTTTAAGGCTTTAATCTTATAAAAATC
TCCAGGGAAAGGTGAAATGCTATAGAAAAGTTAAAGATAGTCTGTGTTCA
ATTATTTGAAGAAACTTGTCATTCAGGGTTAGATTGTTTCTATAGGGTGA
GGACAAATTCCCATTACCTATATAGGTACTTACGTTGTCTTTTCAGGCAA
ATTTGGTTAGCTATCCCCTCCTACCCTGTCAAGCAGTAGCTATCTAGCCC
ACAATAAACTCTCTGAGTGGAAGCCTGATGATTTAAAGTTATGTCTACCA
TACCCATTATTTTTCGACTAACAATAGGAATCCAAAATCATTAGCTTGTA
GCCATGAAAATTCTGAAAATAGACTCTCAGTGTGGCCATATGAGTTACAT
TTCAAAGTGTAACCATCTAGATTTGGGGAAATTAAGGACCTTACGAACAC
ACACACACACACACACACACACACTCATATACACATTATATAGATCTACT
TCACACCAAACACAAGCATAAAAAATAAACTGGGAAAAGATCAAGATTCC
AACTGGGTTTTGAGTTGGAAAAAACATTAGTATTTTAAAATATATGTTTT
GTCAAAGAAAGTGACTATCTGATAAAGGTAAAGGAAAAATCAGCTGTTTC
AGTTTTGGGAAATTCACAATGCTCCTTTGTTTAGTGGGCTTGTTGAACTT
TTCTGAACATTCAAAAAATATTTTTGTCAAATAATTTGGACATTTCAAAT
TCTGCTTCAAGCATGAAGGGAGGTTGCCACTAACACCATTGTTACCTCTT
GGGCCACGGTCTCCCTTGGGATGGTCATTTCAGAGGGAGGAAATACCCTT
GGCTGTCAGAGGCCAAGGGTCTATAAACTTCTAGATGAGGGTAAGCGTGT
TGAAGTCATACTGATTTGGATTTTCATTCTGTCTCCTGTGCTCACAATAG
CTATATGACCTTGGGCAGGTAGCCTAACCTCTCTGAGCTTAATAGCCTCA
TTTTAAAAATGGAAATCATATGTACTCTTCAGGATGATTTTGAAAATTAA
ATGAGATCATATATATAAAATGCTGCCATACAAAAGATAGTCAATGAATG
CTAATTTTGTCTTTCCTTTCCTCATTTAATTCATCTCTCTACCTCCAAAT
ACAGCCTTATCAAAACTAGGTCAGTCATAATTTATCTTATTTTTTAAGAC
CATGGAAGGAAAAAAAGATGGTTTAATCCTTCTCCTAATCCATATTGTGA
CTTCTAACTTTCTCTGGCAGGAAATGATTTCTTAGAACCAACTTCAGACC
ATTACTTTTTTCTTTTTGGAGAAGTTTTACTTTACTGTCTTCAGCAGAAA
AGGAGAGTAGGTGGTCTGGATTCCCCAGATGTGGATCAGGAAAGGCAAGG
TTATGCTGAAGTAATGAACAACCCCAGAATCTTAGTGATGGTTTATAGAA
TAGTTTCTTGTTCATCTCTCCACCCCAAAAAAGCAGATTGATACAACATA
AAGGACAGATAAGGGATAAAGCAAATATACCAAAATGTTAACTGTGAAAT
TCAGGTGATAGATATGGGAAATGTGTGTTATGCAAGTCTTCAACTTTTCT
ATACATTTCAACATTTTTCACGATAAAATATTGGGAGAAAAAGTTTATTT
CTCACTTATGCTATGGGTCCAACAAAAGGTGGCTAGGGTTCCATGCCGTA
TCTTCTCACTCCAGGACCTAAGGTGATCAATCATCTTGAAAGTGGCTATT
TGCCATGGCAAAGGGAAAGAGAGTTCTGTTGGCATTGCACAGACTGTTGG
ATAATTTCAGCCTGAAATGACATGTGTCACTTCTGCTTACAACTCATTGG
CCAGAGCTACCAGCACATGGCCTTAGCCCAGGCCATGGAGGTCAGATCCT
ATCATGTGCCTCGACGGGGGAGGGCTGGAAAGATTTGGTAAGCAGCACAA
GTAATTACCCTGATGAAAATAAACAACTTCATAAACTTCAAAGAATAACA
TACCTTAGGACTATCATTAATAATACGAACGTGACAAGGCAAATTCACTG
GAAGCAATGTCAGTAAAAGAATTCAGAAACTTTTTTCTTTCTCATCCACT
TTCCTTCTTTTAGTCTCCTTTAAAGGGAGCAGGAGAACTAACCCCGTAAA
GGCTAACAGATTGCAAACTTCTTGCTGGCTGGTTTGCAGCCGGCCGGCCA
GCCAGCCAGCCAGCCAGCCAGCTCTTTCTAGAATCTCATCACCTTTTTGT
TGAGTCAGCTCATTTTCCCTGAGTCAGTAAGAAGATGTGGAAGCTGTCCA
GAGATCTTAAGAAAAGCAGAAGTTAGGTTGAGGGTGGTAAAGCTTTTCAG
AAATAGGATAATAAAATATCTGAGTAAACAATATTTCAATATTTTTCTTG
ACTCAACGTTTTAGAGGAGTCAGGAAAGACTTCATAGTGACATTTAAGCT
GGCACTTGAAGGCGTTTGCAAGGTAGGAAATGGAGTAGGGCGTTCCAAGC
AGAGGGAACAGGCTGTGCAGTGGCAAGGAAACTGCTTTGGACTTGGTAGA
TCTGCAGGAGAATAAGAAATTCACTGTGACCAGAGACTAGAATGCCTGTC
AGGATTACTGTTAAAGTAGTAGCGTAGGGCCAAAAATACTGTATTTTTCA
TTTGTTAGCAAGTGGGAAATGAAATACCCTACTTGTTAAGGAAAAATTTT
AGTCTTACCTAGTGCTCTAATAGTAGTTAACATGGCTTCAAAGAGAAAAG
ATTATATGCAAGTAATTTGCTATAAAGAGAACTATAGGCAAATGAGTTTC
TAGCAACTGAATACTTCTGTTTGTTGGCAACAATTGTTGGCAACAATTAA
TATATCCACAGAAACTTTCTTTGGGAAAACAAGTTACCTACTAGGCTGCA
AACAAATTAAGGCAAAACTACCAGGTTATAAAAACAAACACTGCAGGAGA
GTGGGCAGGTAATGAGGTGATTTTAAATGAAATACATTTGTCAAGAGAAC
AGTGATGGGGAGAAGAAAAACAAGGAGCTTTACGTATGATATATCATTTA
ATCCTAAACACATGTTGTGAAGTGAATGTTATTATCACCATTTCATAAAT
GTGAAAACTAAAGATCAAATAAAGTAGCTTCTTATAAAATGAGCTGTTTA
AATTTGAGCCTTATTTTTGAATCTCTGAGACATTTTTCCTCTGTAAAATG
GAGACATGACACATCCTGCCTACCTCACAGGGTGGTTAAGAGGATTAAAA
GAAATAACAAAAGAAATTGACATCATTCTTGACCTTGACTTTTTAATCAG
AAAATCTTAAGTTTTCTGATTCTGTCTTGATGTTTGTATATTCTTCAAAA
CTATGTCTTTATATAGGAAACTTAAGATGGCTGTTTGTGGAAAAAGTCTT
TCTAGCATATCTTAAATATGCTATTTTAAACTATTTTTAAATAATTAAAA
ATTTTACCTTTCCAAGCATCAAAATAGAGTATAACGTTTGGCCATTATCT
GAAATCCATTCATGGAATATTTTAGGAAAATAAGTTTTATTTTTTAAACT
CCTTTAGTTTTTCTCTTGGCATTTTAAATAATTTTGTTTGAAAATGTATC
GCTCCTTTTCTTTTTTTCTTTTTTTTTGAGACAGAGTCTTGCTGTGTCAC
CCAGGCTGGAGTGTAGTGGCAGGATCTCAGCTCAGTGCAACCTCCACTTC
CAGGGCTCAAGAGATCCTTCCACCTCAGTCTCCTGAGTAGCTGGGACCAC
AGTTGCACACCACCACGCCCAGCTAATTTTTGTGTTTTTCATAGAGACAT
GGTTTGGCCATGTTGCCTAGGCTGGTCTCAAACTCCTGGGCTCAAGCGAG
CCACCTGCATTGGCCCCCCAAAGTGCTGGGATTACAGACATGAGCCAACG
TGCCCAGGCTCCCTTTCTTTTTTAATGAAATATACCAAATAATGATATTA
TGTGCCAGGAACTGTTCTGAAGGTTTAATGTACATTAACTTATTTAATCC
TCATAACCATTCTGCAAGGTAAATACTATTATCTCCATTTTCCAAATGAG
AAAATTGAGGCACAGAGGTATTAAGTGACTTGCCTAAGACCACACAGCTA
ATAAACAGCTGAGCTGGGATTTAAATTGAGGTATTTTTGCCCAGAATCTG
AACTTTTTTTTTTTTTTTTTTTTTTTGAGAAGGAGTCTCCTCTGAAGCCC
AGGCTGGAGTGCAGTGGCATGATCCCGGCTCACTGCAACCTCCTCCTCCT
GGGTTCAAGTGATTCTCCTTCCTCAGCCTCCCAAGTAGCTGGGATTACAG
GCGTGCACCACCATGCTGGGTAAATTTTACATTTTTAGTAGAGACGGGGT
TTTGCCATGTTGGCCAGGCTGGTCTTGAACTCCTGACCTGAAGTGATCTA
CTCGCTTCAGCTTCCCAAAGTGCTGGGATTACAGGCGTGAGCCACTGCGC
TTGGTGGAGAATCTGAGCTTTTAAACACTGAACCATGATACATTACTGAT
GATAATGGGCCCTCTTTCTAAATGAATCAGGAATATCATTAGGCTTATCA
ATTTTTATTTGATATATACAGAAACTACTGTGGTTTAATACAGGATTATT
TATGTACCTACAGGATACAGTTGTCCCCATCTACTGTTCTTTTGATTTAC
TTTGTCTGCTTTTTATAGTTTTCTCTGTCCTCTCTGAAAAAAAAAGAATA
TTTTCAGTATATCTGCCTCTACAAATTGTCTTCTTCCCTTTCATGTTCTA
CTTCCAATTTCATAAGAGTCAGAAACCAGATCACCCCACATGTTAGAATT
GTGTCTAAAACAGGAGTCAAGAGGCCTTGGGAGAAGACCCGAGCCTTTAG
TGAATGGACTTGGTGACATCTGTGACAGGGATCTTTATTTTTTGGCATGT
TTTCTGTTCTTAACTGTGATCCAGATAGTTTAAATGGCTGGATAAGTGAC
TTCTCAGTAAGCGAGGTGTCACAAAATATAAACTCAAGGCATTTCTATTC
TTCCACCTGTTCAAAAGTGTTTTAGTCATCATCATAATTGGAAGGATGGC
CTAGTTCCTCAAGCTTCTGGGTAGCCATCTGATGCTTGGAGGTAGAGAGA
CGTGGCTACAACTTGTATAAAAATAGCCCTTCCAGATGGCTGAGGGGCAA
AGTTACAACTCAGCAGCTCGATATAGCTGAGCACTCAAATTTTGGAGTAG
GAGGAGAGCTATGTGAGAAGTAGCCAGAGAGACTGGGCTGGAGCCCAATC
ATAAGTAGAACAGTATATGCCAGGAGCGATCACTTAACGTGCAGATTAGC
TGTTAAGTTTTTCTGTTACTTCATCTCCATCTCCTCCCCAACTCCCTTGA
TTCAACCTTACTTCTGGCCCTAAGGAAACACATACACATACGACATAGAC
AGATATGCACACACACTTTCCCCTTTTTTTTTTAAAGGAGTATAAAATAG
AGGAACAAACCATAACTCTTAAAAGGCTTCAGAATTCTACTAGTCTAACC
CTTCATTTTAGAGATAAGCAAAGTGAGGCAGAGAAAGGTGTGGTGACCAT
CCAGGATGTGAGCAGGACTCCCACGCTCCAGGTGCTCTGCCAGGGCTCAT
CTTCTGGCTCGTTTGTGGGACACTGATAACAGCGCAGCTATCCCAGGACA
TGTTTCCTCCAAGTCTGCTTTGACTCATTTCAACTCTAATAAAGCCTTTA
GTTTAAAAAAGAAAAAAAAAAGTATGGGCCCAGCGTAGTGGCTCACGCCT
GTAATCCCAGCCCTTTGGGAGGCCAAGGCAGGTAGATCACGAGGTCAGGA
GTTTGAGAACAGCTTGACCAACATGGTGAAACCCCATCTCTACTAAAAAT
ATAAAAATTAGCTGAGCGTGGTAGCACACGCCCGTAATCCCAGCTACTCA
GGAGGCTGAGGCAGGAGAATTGCTTGAACCTGGGAGGTGGAGGTTGCAGT
GAGACAAGATCATGCCACTGCACTCCAGTCTGGGTGACAGAGTGAGACTC
CATCTCAAAAAAAAAAAAAAAAAAAAAAGAGTGGATTATTGCAAAAGTCA
TGATAATGTCTCCACCCTCAACACAAATACTCAAGCTCTAGTCCCTGGCT
TGAAAACCTGTTTCACCAGTGAAAATATTTACTAATCAAATTAGTAAATA
TTAAATTGTGTAGTTCCCACTTTTTATTTTAAATCTTTTATTCACTCTGC
ATTTCTATTGGATATTCTGGAAGAAAAACCTCTGTAGTAAATCCATTGTT
TTAAATATCTCAATGTCTGTTCAAATAATTGAGTGTTGGTTTTAAAAAAT
AGTCATTAGGAATGGGTTAGTACTTACGTTTTAGCAATTGTAATGAAGAC
TTTAAAAAAAAACAAAAAAACAAACAAAAAACACAAGCTTATTGAGAAAC
ATGTTACACTCCTTTAACATCATTTATGATCCCCAAAGGCAAGGCATTGC
TGTTCCCTGGTGGCTGTACAGCATCACTGCAGGTAAGCGATTTGGAGGTA
GAAAAGGTGTTGAGTGCCAGCCTTTCAAATTCAGTTCTACAATGAATCAA
CTTTTAAAAATACTCCCAGTAGCTTGCATTTTCTTATAAGAAGGGTTTAC
AGGTAGCAAAAACAAACAATCCCCAACGAACTTTTTCCAATACTATAGGA
TTTGTGCTGCCCAAGTAAATATTACATTATTGGAAAATAGCATGTTACTT
TTCAAGAGGAAGTTCACTTCCTTCTTATGGAATTTATCCTTAAACTGTAT
GGTATATGGGCTTTGTGCAAGGTGCTGGAAGTAGGCAAGGAGGCCTCAAC
CATGATGTACACACACTATTATAGCACCATTGCTCTCCTGCTACTGAGTC
ACATAGCTCACCAACTGGGGAGGGGTCACCACAGAAAGAGTAATGGAGCG
AGATAGGTACAAGCATTTTTTGATTGCCCGCCAAGCACCTGGGCCTGTGC
TAGCCCTGTTAAACTTGGGTGGAGCAATCCTGCTAAAAGGTCAGGCTTGA
TTCATGAATAGGTGTGCTAATTCTACTCCGGATACATCCCCGTCATCCTG
TAGCATACAGGCCTGAGTGAAGCTGGCCTAGAGCAGGGATTCACCTGCTA
GATAATTATTGCCCAAATTAAGAATGCAAGTCAGGGCATGATTTTCAACG
CCTTTATTAAGAAATATCAAAAGTTATTACAGGTCCATATGCAGTTTTAC
AAAGTTCAAGTGAAGAAGACTGTAGGGATGCCATCAATGTGCGTGTCTGA
AGACTATGGAAGCTTGTCAAAGGGGTAACCCTACAACTCCTGTCACTTTA
ACAATGGTCCACAGCAATGCTTTTCCCCCATTTCTACTAGGCTAGGCCAT
TGCACAATACCTTAAGCTACTTAAAAGAGTTTTAATACGTTATAAATACG
TACATATTTGTCCTTCTAGTTTGTTACCATCCTTCCCTGAAAGAGCGGAG
CTGTTTATAGGAAGCACAACATTTGAGTCCCATTCACTGCTGTTTGTATT
ACATTTTCACAAAGCGTGCTTTGAAAGCTGGCAAACACTGCAAGTTACAT
GTTACCATATTACACATGGGAGGACCCATACACATTGTTATTTTTAAATT
TAACATTGAACAAAAAAAAGGAGGAATCACTTAACTTGGAAACAAAACAC
CAAAGTTAGTCAGTGGAATGGTCAAGAGCAGGACAAGCCTGCCAGGGCCA
CTGAACTCCAGTCACAAGTGGTCAAAAGTATGACCGAGAACAACTGGATG
CAGGAACATCAGCACAAAATACAGATGGGAATATTCTCGGGAGTGGTAGA
TCACCATCTTGTATGTGAGGGATATTGTGTCAGAGGGGAATTACACTTTC
AAAATAACCAGGGCTAGTAGGTCATTAGTACCAGATGGGGAACAGCAATG
GTGTGGCATGCCTTGTTTAAGGCCTGCGCTGGTTTATCTAAAAGTAGATT
TCTGAAAAGATTGTCCAGTAGCATCAGAAAGAGCCACTTTCAGAGGAAGG
ACTCCTTGTTCACCCACATCAGACTGCGTGTCTCGTTGGGCTTGCATTCA
TACTCAATGACAGAGAAGGGGCTTCCCCACTGGCAGTGATCCCGCTTGTG
GTAATTGTAGAACTTGACTTGCCAGACTGTCTCGAAGGTCCCAATGTCTG
TGAACTGAGGGAAGAGAAACATGCCTGATAAAAAACCTCATTCTGCCACG
AGCAATAGATGAGAGCTAACATTAGTGATGTAGCTCCTGGATAATCAAGC
AGACCCAGTCCAGATTCTCTTGGTCTGCAAGGCTCGGAAGGCTTTCCTTT
AGTGGTCTAGCTCCCTCCTTTATGATGGGAGATATTTTCCTAAAAAGCTG
TCTGTAAACCATTCTTCTCTATAAAATCTGATTCCCATTGCAAACGTGCC
ACTCGCTATCATATTGCTTTTAATAAAAATAACTGCAAAGTTTATCTTGT
TTTTCAAGTTTAATTTTTTGAAAACTTAGAATTTTAAAGCTTACTCTAAA
ATGGCCCCTGCTGGCCAGGTGCAGTGGTTCACCCCTGTAATCCCAGGCTG
AGGCAGGATGCTAGCTTGAAGCCAGGGGTTCCAGAATAGCCTGAACAACA
AAGCAAGATTCTGTCTCTAAAACAAACCCCCATGTTCATCCCCCAAATGG
TCCCTGCTACAGAAGGGGAATGGCGAAGGGAAGAGAAGGAATAAAGTTTA
CTGGCTTTTCTCAAAATACAATGGTACAAAACAACCTGTCTACAATACAT
CTACATAAAATTTCCATCATTAGTTTCAATTCCAAAGAAAAATATTTCAA
AGTGATAACTGAAAATATTTCATTGGAAGTGTTAAAGGCCATATTTTAAC
TATTAAACATTGAATACTTATAAGTCAGGAGTCCATGCCTACATCTTGGG
CTTAGATTATACATTACCCAGAACCATATTTGAGTATCTGCTATGTACCT
TGCATGTATAAAAATTCGTATAAGGCAGGATTGTTGCTCTTAAGAAGTTA
GCATTCTACTTCCAGAAGGTAGGCCCTGTAAATCACTGCACAGAACTCTC
CTGAGGTATAAAGTACACATTTCTCTTGCAGCTGAGAAGAGTACCTGGAA
TACCCTGGAGGCCACTGGGCAGATGCTCAAGTGGCCTGTGGGGTAGCCTG
ACCCTGCTACTGCCTTCTGGAGCTATTTAGGGGATCTGGCATTAAACTGC
ATGATGCCTGTCGTTATTTTCTCCTCTGACCCTCCCCTGTCATGGGAGGG
GACAGGGACTGACTTGGCAGGACTCTTAGACTTGTCTGGAGCCAGAAGAC
GTGGACAGAGGAAGGCAGTGAGCATTAGGAGTCTTATGCTTCTTGCTGAT
ACAGGAGGAGGGTGATCAGGTGCTTTCCAGATAGCTTTCCAAGATGCTCT
CATTAATTATATCTTGCACCATTGCCTCATCCTGCTAATTTGATCCCAAA
ACAAGATCTAGGCTTTCCTCTGGGAAATTTGCTTGATTTGAAATAGTGGA
TGATATACCTAGAGTCCACAGACCTCCCAGGTATGAAAGTGGATTACCTC
TTTTTAGAGAGTCATGGCTTAAAGACCTATATAAGCATACAGCAGCTTTA
TTCACAATAGCCCCAGGTTGGAATTGACCCAAATGTTTATTCACATGTTG
ATAAGCAAGTTTTGGTATATTTATAAAATGGCCTTTTGCTGAACAATAAA
GGAAGACATGCTGATTCAAGCAGCAACAGGGATGGATCTCAAAGACATGC
TAAGCAAAATAATCTAGGCCCCCAAAGTACATGCTGTATGAATCCATCTA
TATAGTGACAGAATGTAACCTACACTGACAGGAAACATCTGTGGTTATCT
AGAGCCTAGGGTGAGAAGGGGAACTGATGCAAATGTGCAAGAGGGAACTT
TTTAGGGGTGATGGAAGCATTCTGTATCTTGATTGGGATGGTGGTTACAT
GGCTGTACATATATGTTGGTCAAAACTAATTGAACTGTATGCTTAAGATG
AGTCCATTTATTACATGTAAACTATACCTCAAAGTTAAGAAAAATAGAAA
TATCCATTTTTAGTGAGCTATAAGAAAACAGGTATTCTAAATGCTGCTAA
TGGGAATTGAATTAGCCTTTTGGAAGGATCATTTGCATGAAGTTAAAGGT
GTGAGTACATTCAGGCCCAGCAATTCCATTTATAATTTATCTTAAGAACA
TCACAAAGTTTAGAGTAGTAAACAATGCAACACTAAAGGTCCATCTTTAA
GGCACTATTTGTGGCATATCTATATAATGGAATACCATGTAATATTTTTA
AATGTAAAGTATTTAATACAGTTTTTATTTTATATTGTTACATGAACTAA
GCAGAATACCATGAAGAACATGAAGTATTAATTCAGTTAAAATTAAACTT
GGCTAGGTGCAGCAGCTCACACCTGTAATTCCAGCACTTTGGAAGGCCAA
GATGAGAGGATCGCTTGAGACCAGGAGTTTGAGACTAGCCTGGGCAACAT
AGTGAGACCCTGCCTCTACAAAAAATGTTTGAAAATTAGCTGGGTGTGGT
GGCACACACTGTGAGACTGAGATAGGAGGATTGCTTGAGCCCAGGAGTTT
GAGGCTGGAGTGAGCTATAATCATGCTACTGCATTCCAGCCTGGGCAATA
AAGACCCTGTCTCAAAAAAAAAATGTATGTGTATATATGTGTATGTGTGT
GTGTGTGTGTATTTAATGTTAGAAATCCTGATGGACTGTTTTATTTTCTC
CTATGTTCTTTTTTTAATTTTTATTATATTTTTGAGATGGAGTCTAGCTC
TGTCGCCCAAGGTTGGAGTGCAGCAGCACGATCTCGGCTCACTGCAACCT
CTGCTGCCCGGGTTCAAGCAATTCTCCTGCCTCAGCCTCCTGAGTAGCTG
GGATTACAGGTGTGTGCCACTACGCCTGGCTAATTTTTTTTGTATTTTTA
GTAGAGACGGGGTTTCACCATGTTGGCCAGTCTGGTCTTGAACTCCTGAC
CTCGTGATCCACCCACCTCGGCCTCCCAAAGTGCTGGCGTGAGCCACCAC
ACCCGGGCCAACTTCTATATTTTTTAGCAGAGACGCGGTTTAACCATGTT
GCCCAGGCTGGTCTTGAACTCCTGACCTCAAGTGATCCACCAGCCTCGGC
CTCTCAAAGCGCTGGGATTACAGGCATGAACCACTGCACCTAGCAGATTT
TCTTCTATATTCTTATTTGTATTTTCAAATTTAACGATTACTTTACCAGA
AAAAAAATTAAACCAAAACTGTATTTCCCAAAAGTATTACCAAATAGTCA
TTATCCTTAATATAAAGGACCATATGCATTATTAGTAAGTACTACGAGGA
ACATTGGAATAAAAAATTGGAATGGTTCATTAAAAACAAAACAAAACAAA
GCAAAAACCATAGGCTGGGCGCAGTGGCTCATGCCTGTAATGCCAGCACT
TTGGGAGGCCAACATAGTAGTACCACTTGAACCCAGAAGTTTAAGACCAG
CTTAGACAACATGGTGAAACCCTGTCACTACAAAAAATTAAAAACATTAG
GTGGGCATGGGAGTGTGTGTCTGTAGTCCCAGATACTCAGGAGGCTGAGG
TGAAAGGATCGCTTGAGCTCAGGAGGCAGAGGTTGCAGTGAGCTGAGATC
TTGCCACTGCATTTTAGCCTGGGCAACAGGGCAAGACACTGTCTCAAAAA
AAAAAAAAAAAAAAAAAAAAAAAAAAAGGAAAAAATGACTAACACCTGCA
AAAATATTCAAATGTAATTAGTAATTAAAGAAATATAAACTTTTAAAGAG
ATACTATTCCTCTAACAACTTAGTGACAATTTCTGAGTGCCAGAAACAAT
CTAAGCATCCAACATTAGGAAAATATGAAAATGGTGGCATATCCATATGA
TAAACTTACAGAGCAATTAGGAATTTAGGGTTGTCGATGATTCAAGGAAA
TGCTTAGAACAATGTTAAAGGAAGATGAATTGGAATGGTTTATATACATT
ATGAGCTCAAATATGTAAATAAGATGTATAGAAAAAATGAAAAATATTAC
CTTCTAAGTAAAGAAATTGAATTTTTCTATCCTTACTCAGATTTTTTCTA
CAATAAATGTTACTTTTAAAATCAGAAATTTTTTAAATGGTGAAATGGAT
TTATCATCTTACAATAATATATACAGCCTGCAAGCCTAAGGGATTTTATA
AATTCTATTAAAAATATGTACATTCTAATGAAATTATGACTTTGTTCACA
ATAGGTTAGTGGTGGTATGTATAAATCAAAATCTCATTTTATAATTTACC
AAGACTTTTAAAGAGGATTTAGTATTATTTTTGACCAGCACAAGGCATTG
CACACTTTAAGTGCAACTGAATTTTACAATTCAATTAACATTTTAATTCC
AAATTATAAAAATATTTTGTAATAAAATATACACTGAAATATAAACTTTA
TACTAAAATATTTTATAATACTTTTCAAATGCCAGCTAATATTTTGAAGG
AAGCAGCTTAAAATCATGCTAGGTAACAGAGAAACAACACTGAGATTTTT
ATTTTTTGAAATAAAGTTGGTTATAAAAATAAAAAGCCCACCAAGCTGAT
TACAACCAGACAAGAAAAACGTAAGGCTTATGTCTAAAAAAATTTTCAAA
AGTCTCAATCTAAGAATTCTCAGTAACAAAGGAGACCAAAGTTAAAATCT
GCCAATAGCAACAGTCATAGCTAAGATATATTTAAGTGTTCATATTAATA
TTTCAAATTTGGTTTATTAATGTGGGACCTAAGGTCACGTTGAAGTAAAT
GTAACTGCTGGTAACTATGCATATGTCTTCCTCTCCCCGATGCCTTAGCT
GTTTATACTTCTCATTAGCCGTCTTCCTATTCTTTTCTTTTGGAGCTCTA
TTTCCTGGGCCTCTTACTACCTAGCTCATAATTTCTTCTTGTCTGGCTAG
AAACAGATGGGGTCTTGAAAGCTGACATTCTTCTTAAAGAACAGGAACAA
TTATACAGGCTCTTCACTCCCTCCCCGCCCATCTGTCCCTTCTAAGGAGA
ATGTACCCAGGCAGGCTCCTGGGTGGTACAATTTATGCACAGGTATAATT
CTGTATATTTGTATAGCACCGTATTTTATGTGATACTTTCACATACATCA
TTCCACTTTAGTGTTGTGCCTGGTTTTATTATCTCCAGTTTATGGACAAA
AAAACTAAGATTCAGAGAGGTTAGATGATTTGCCCTCAGTCTAGACTCCA
AAGCCAGCCAATGATAGGAGCAGGACAAGAACCTAGGTCTCCTAATTCCT
AGCTTAGGAGCTATTTATCACTGTACAAAACTGCTGATATAAATGGTTAT
AGTGGACAGTTTTTTTTTCTCCCTGAATGAGAAATTACATGAGTTGTCAC
AGGTAGAATGCTTTAGGGCATTGCTATGGACTGAATGTTTATGCCCTCCT
CCAACCCCAATTCATGTGGTGAAGCCTAAATCCTAACTGTAATGGTATTT
GGAGGTAGGGCCTTTGGGAAGTATGAGGGTCATGAGGGTGGAGCCCACAT
GAATGGGTTCAGTGCCCTCATAAGGTACATGAGAAAGATGATCTCTTCTT
CTGCCGTATGAGGATACATCAAGAAAGTGTCTATGGACCAAAAAGAGGGT
CCTCACCAGACTACTGCCTTGATGTTTGGTCTTCCCAGCTTCCAGAACTC
TGAGAAATAGATGTTTGTTGTTTCATCCATCCAATCTATGGCATTTTGTT
TTAGCAGCCCCAGCGGAGACAACACTCTACCTGGACATTTTATTTGCTGT
AATGTTCTTACGAGTTGCAAGCCTGGTCCACTTTTTACTTCACAACTAAT
GTGGAAGAGAACACATCTGATTCCAAGGAGAGGAGACTCATCTCTTGTCC
TCAATAACAAATGAGGAATCTGAAGAGCTTGTTTTAAACAATTTTGGTGG
TCTTACAACCCACTGGCATAGAATCTCGAGTGTACTTCTTTCTATTCACC
ATGCCACCTACTTCTACTGCAGACATGAAATTAATCAAGATCACGTAGAA
ATCCATTGGCTTGGGAGGACAGAACAATCTTATTCATAGAAACATGATGT
TACTTTATTAGTAAACAAATTCAGTATAAATATGCATCTGTGACTTTTAG
TGTGGTGTGACCTATCAATTTCCACTGAATTTCAAGCCTTTCTATTCCTG
AATTGTTTGAGGTTTTATTAGTAAATGTTTCCCATTTCTCTTCACTAAAT
AGGACTAAAAGTTCTAGGTCTTATAAAGTACATTTGTCTTTATAAGCAAC
TGTTAAACTTTTCCTAAGTGATTGTGCCCATGTTATATTCTCACCAGCAA
TGAGAATTTCAATTGCTCTATATCCTCTCCAACACTTGGCATTGCCAGTC
TTTCAATTTTAGCCAGTCTGGTGACTATATAGTGATAAGACAAAGAAGGA
AATCAAGAGGCCGGGTGCAGTGGCTCATGCTGGCAATCCCAGCATTTTGG
GAGGCCAAGGCAGGTGGATCACAAGGTCAGGAGTTCAAGACCAGCCTGGC
TGAGATTGTGAAACCCCATCTCTAGTAAAAATACAAAAATTAGCCGGGCG
TGGTGGTGGGTGCCTGTAATCCCAGCTACTCTGGAGGCTGAGGCAGGAGA
ATTGCTTGAACCTGGAAAACGGAGGTTGCAGTGAGCTGAGATCACACCAT
TGCACTCCAGCCTGGGCATCAGAGCAAGACTCTGCCTCAAAAAAATAAAA
AAAGAAAAAAGAAAAAAGAAACAGCAGAAATCAATGAATTAGAAAAGAAA
GACTATAGAGAAACAATGAAACTAAAATCTAGTTTTTTTGAAAATATCTT
TAAATTGATAAACCCCTAACAAAACTGAGCAAGAAAAAATACAAATATTA
GGAATGAAAAAGAGGACATCACCATGGATCCTACAGACCTTAAATTAGGA
AAACCTTATACTAATAATTTCAACAAAATGGACAAGTTCTTCCTTGAAAG
ACACAAACTATGAGTTGACATCTACCTAATGAGGAAATTGAATTTGTAGT
TAAAAATCTCTCAGGCCCAAATGGCTGCATTGATAAATTCAGACATATAA
GGGGAAAAAGCCACCAATTCCACATAAATTTCTTTCAGACAGCAGACAGA
ACAGTTCTCAAATAATTTTATGTAGCCAATATTGCCCTAATTCCTGACAA
AAATATTACAAGAGAAATTACAGAGAGATTCCTCATGGGCATATACGTAA
AATTTGTTTATAATTTTTATTTGCTTGAAGTATTGGTAAATCAAATCTAG
CAATATATACTGAGAATAATATATCAAAACCAACAGGGATTTATCTTCTG
AATTCATAAGTGGTTCAACATTTGAAAATCTACCATATTAACAGAATAAA
GGAGAAAAACCATATAATTATATCCATAGTTGGAGAAAGAGCACCTGACA
GTGCATCATTGATTCTATTCTCAGCAAAGGAAAGTTTTTCAACTTCATTA
AGGGGCATTGACAAAAAACCTATAGCTAACTTTATCCTTAATGGCAAAAG
ACCGAGTGGTTTCCCTGTCAGATGGGAAACAAGGCAAGAATATGTGTTCT
TACTGCCCCTATTCAGCATTGCATTCCAGGTCTTAGCCAGTACCTTAAGG
CAAGAGGAAGAAAAGGGCGGTGGGGAGAGTAAAACGGTCTATCCAGTGAA
GAACTGTGTCCGCTAAAAATACTAAGCAATTGCCTGGTGAGGTGGCTCAT
GCCTGTAATCCCAGCACTTTGGGAGGCCGAGGCGGGTGGATCAAGAGGTC
AGGAGATCGAGACCATCCTGGCTAACACAGTGAAACCCCGTCTCTACTAA
AAATACAAAAAAATTGGCCCAGCGTGGTGGCAGGCACCTGTAGTCCCAGC
TACTTGGGAGGCTGAGGCAGGAGAATGGCGTGAACCCGGGAGGCGGAGCT
TGCAGTGAGCCGAGTTTGCGCCACTGCACTCCAGAGCCTGGGCGACAGAG
CGAGTCTCCGTCTCAAAAAAAAAAAAAAAAAAAAAAATACTAAGCAACCT
TAACAAATAGCCAGTAGAATAAGTGAATTTAGCAAGGCTTCAGGATACAA
TATTAACTTATAAAATTCATATTTCATAAAGAAATATGCCAGCCCTGAAC
AATTATCTTTATTATTTCACCAATTTTACATGAAAGTTGACATTGTATTA
CTTACCAATAGTGAGATAAAACCATTTTTCATATATTTATTGGCCACTTG
TATTTCTTTTATAAATTGCCTATTTATATCTTTTGCTTAATTTCCTACAT
CTTATGATTATCTCTTACACATTTCTAATAATTTGTTGTATAAAGTCACA
GCTTTCACCAAATACTAGACCAGACAGCTGTCACGAGGACCAACTTCTAT
ATTTGGAAGGCTATTTCCCTCCACTCACACATACTCAGCCTCATTACCAC
CTCATATACCTCCCTGCCTTCTTCCTCCAAACTCTCACCAGTCTCCCAGT
GAAGAGGGCCAACAAAAGGGATTATGATGATGATTCGTGCAGACAGACTC
TCTATCTGGCCTTTCTCCTTGGAGGGACTGATTGGGAATTAGTCACACTG
CTCTGTGAAATGGGGTTTGAGAGGCATAAAGTTGGGTGTCACTTTATTAG
CTCCCAGTACCAGGGCCTCTAGAATTATCTGAAGATCATTGCCATTAATC
TAAGTCAGATGGCATTTGACTATTTCCTGACTTCAGTGGCCACTCACAGC
TTCAGCAGGCAGTAAAGGGCTGTGAAATGTCATTGACAGTAGCTGCAGTG
TCACTATGGGCAGTGGCAAATCAAGGCAACACAGCTTAGGAGACTATACA
TGTTATCAATGATTTTTTCACCAGTTCACTTTGAAGTGTGATATCAGAAA
AGAACAGTAACGTTTCCCCCAAATAGCCAGTTATCCCAGAATCATTTATT
CAATGATTTGAAAAACTTTTTTTTTTTTTTTTTTTTTGAGACGGAGTCTC
CCTGTCGCCCAGGCTGGAGTGCAGTGGTGCGATCTCGGCTCACTGCAGGC
TCCGCCCCCCGGGGTTCACGCCATTTCTCCTGCCTCAGCCTCCCGAGTAG
CTGGGACTACAGGCGCCCGCTACCTCGCCCGGCTAATTTTTTGTATTTTT
AGTAGAGACGGGGTTTCACTGTGTTAGCCAGGATGGTCTCGATCTCCTGA
CTTCGTGATCCACCCGCCTTGGCCCCCAAAGTGCTGGGATTACAGGCGTG
AGTCACCGCGCCCGGCCGAAAAACATTTTAAACTACTTGGTATCAACTTT
GGAACTCTTAAGCAAGACTCAAAAGACAATAATAGCTAGGATATGTCATC
AGAGTTGAAAGGAAATATTATCTTAAAGCAGAACTGCAAATACTAAAATT
GCCAGAATTTTAATGAGGTCTTAGAGGGCTAATAAGCTACCTTTCACTTA
AAATATCCTGAGCACTGGGTAAGTGTGGAGAAGGTCATCATAAATGAGAC
TGTTTCCTCTCTTCCAAGTACTCAGAGGGCAGAGAGGAGACCTCCCCAAC
TCCTCTCTGGAGCTGACCTGGCTTGGGGAGACCTGAGCTGTAATTTCCTG
TTCACCTCCTCATGCTACTACTGCTCCCTCTGGGCCTCTGGTCTTAGCAG
GTTGGATTCACACTTCTTTTGCTTTAGATGTAAACAGAACACTAGGAAAG
AAGATTAATTCAAACAGTTTCAAAAGCACTCGATTCAAAACTTCTTCCTA
TTTGGATCTGTAAAGTAAGAATTTCTATATCTTTTTGAATAGCTGAAGCA
CAGGGTCTTCTTCTGTTTTAATGTGAAATGATTCAGACTTAGTCATTAAA
ACAAAGATGTTATTACCTACCATAAGTCTTATTTGCCTGTCCAGGCATCC
ATTAAAAGGTTGGAGAGTCCAGGCGCGGTTGCTCACCCCTGTAATCCCAG
GACTTTGGGAAGCCAAGGCAGGCAGACCACCTGAGGTCAGGAGTTTGAGA
CCAGCTTGGCAAACATGGTGAAACCCTGTCTCTACTAAAAATACAAAAAA
TTAGCTGGGTATGGTGGTGCATGCCTGTAATTCCAGCTACTTGGAGGCTG
AGGCATGATAATTGCTTGAACCTGGGAGGCAGAGGCTGCAGTGAGCCAAG
ATCACACCACTGCACTCCAGACTGGGCGACAGAGCAAGACTGTCTAAAAA
CAAACAAAAAAAACAAACAAAAAAGCCAGCCAGGTGCAGGGGGTGCATGC
CTGTAATCCCAGCACTTTGGGATGCTGCAGCAGGCAGATAACCTGAGTTC
AGGAGTTCAAGACCAGCCTGGCCAACATGGTGAAAACCCTGTCTCTAGTT
GAAAAAAAAAAAAAATACAAAAATTAGATGGGTGTGTTGGTGGGTGCCTA
TAATCCCAGGTACTCAGGAGGCTGAGGCAGGGAGAATTGCTTGAACCTAG
GAGGTGGAGTTTGCAGTGAGCCAAGATCACGCCACTGCACTCTGGTCTGG
GTGACAGAGTGAGACTCCATCTCAAAAAAAAAATAAAGTTGGAGAGCCTT
AGCTTAGTTACTAGATGATTTGAAGGTATGTTTTAAGAAAAAGGTCTGCG
GTCCCTAACTGTAACTCGTGAATCCAGCAGTAAAGGCTAGGTTTCTATAT
GTGAATTTTCACATCTAGAAGCAGCAGCTGATTGACAGTTGATTGTTCCC
CACCATGATCACATAGGTTTTGGTTTGAGCACTGGTCATCTGGTTGTCTT
TTTTTTTTTTTTGAGACAGAGTCTCGCTCTGTCACCCAGGCCGGAGCAGT
GGTGCGATCTCAGCTTACTGCAAGCTCCGCCTCCAAGGTTCATGCCATTC
TCCTGCCTCAACCTCCCAAGTAGCTGGGACTACAGGCGTGTTTGTATCTT
ATTTCTAATAGGCTGATAATAAAGAAAATGATTGAAAATATAGGTTTTCT
GATCAAGTCACCAGCTCCTATATTAAGTTCAAATTTTTATTCTAGTCTTG
AAGCTTTCAGATAGCCAACACAATCCAATGACATTTTAAAGTCACCAACA
TTTCAAACTTATGTATTAAAAAATATAGACACTGGGCCGGGTGCAGCAGC
TCACGCCTATAATCCCACCACTTTGCGAAGCCGAGGGGTGTGGATCACCT
GAGGTTCGGAGTTTGAGACCAGCCTGACAAACACGGAGAAACCCTGTTTC
TATTAAAAATACAAAATTAGCCGGGCATGGTGGCACATGCCTGCAATCCC
AGCTACTTGGGAGGCTGAGGCAGGAGAATTGCTTGAACCCGGGAGGCAGA
GGTTGCGGTGAGCCGAGATTGCGCCATCGTACTCCAGCCTGGGCAACAAG
AGTGAAACTCCGTCTCAAAAAAAAAAAAAAAATATATATATATATATGTG
TGTGTGTGTGTGTGTGTGTGTGTGTGTGTGTGTGTACATATAAAACATAT
ATAATATATATTTAAATATATATAGACACCACACTCTCATGGACAATTGT
GAAAACTTGACATGTCCAAGTAAATCCAATCAATTAATTAGCAAGTCCCT
GGATTATTCAGATTTCGTCAAGGGAAATGTCATCAGTGTGGGTGCCTAAG
TCAAGTCAGAAATGTCAGAATTAAAACTGGAAGTAGGCCGGGGGCGGTGG
CTCACGCCTGTAATCCCAGCACTTTGGGAGGCCAAGGCGGGCAGTTCACG
AGGTCAGGAGATTGAGACCATCCTGGCTAACACGGTGAAACCCCGTCTCT
ACTAAAAATACAAAAGAATTAGCTGGGCATGGTGGCGAGCACCTGTAGTC
CCATCTACTCGGGAGGCTGAGGCAGGAGAATGGCATGAACCCGGGAGGCG
GAGCTTGCAGTAAGCCGAGATGGCACCACTGCACTGCAGCCTGGGCGTCA
GAGAAAGACTCTTCCAAAAAAAAAAAAAAAACACACACACACACACCTGG
CAGTAAAGAAGATATTAAGGCTGGGCACGGTAGCTCATGCCTGTCATCCC
AGCACTTTGGGAGGCCGAGACAGGAGAACCACTTGAGGCCAGGAGTTCAA
GACCAGACTGAGCAACATAGCAAGACCTTGTCTCTACTAAATATAAAAAG
GTTTTGCTGGGTATGGTGGTGCGTGCCTCTGGTCCCAGCTACTAGAGAGG
CTGAGGTAGGAGGATCAGTTGCCAGAGGTCAAGGCTGCAGTGAGCTAGAA
TCATGCCACTTCATTGCAGCCTGGGCAGCAGAGACCCCACCTCTACAAAA
AAAAAAAAAAAAAAAAAAAAAAATTAGCTGGGTGTGGGTGGTACGTAGTG
GTAGTCCTAGCTACTCAGCAGGCTGAGGTGGGAGAACCGCTTGAGTCTGG
GAGGTCAAGGCTGCAGTGAGCTATGATTGTGCTGCTGTACTCCAGCCTGG
GTAACAGTGAGATAACAGCCTGGTAATAGTGAGATCCTATCTCAAAAAAA
AAAAAAAAAGGAAAAAAAAGAGTACCAGGATACTGTTTACCTCTAATTAA
CTGTGTGATCCTAGACATATCTGAACTCGTCAGGCCTGAGTTTTCAATGA
AAAGTGTATTCCACTATCTAATCTCTAAGGCCACTTTTAGCTCTATCATG
GTATAAAGATTACAAATGGCGACCAGGCACAGTGGCTCACGCCAGTAATC
CCAGCGTTTTGGGAGGCCAAGGTGGGAGGATCACCTGAGGTCAGGAGTTT
GAGACCAGCCTGTCCAATGTGGTGAAACCCTGTCTCTACTGAAAATACAA
AACTTAGCTGGGCGTGGTGGCGGGTGCCTGTAATCCCAGCTACTCGGGAG
GCTGAGGCAGGAGAATCGCTTGAACCTGGGAAGTGGAGGTTGCAGTGAGC
CGAGATTGCACCATTGTACCACAGCATGGGTGACAGAGCAAGACTCCATT
AAAAAAAAAAAAAAAAATTACAAACTTACCCTTAAGGGTATTGCATTATG
TATTTTTTGAAGAAAGTCAGATTCTCGAATACTGTCATTTACAATCACAC
CAAACAGGACAGAAGGCCATTCAAGTACCTGTAGAATTCCTTTTTGGTTG
GGCACAGAGGGGAAGGCTAGGAATGTGTCCCAGCATCTTAGATGATTTGC
ATGATTTTAAAAAGTGAAATAAGATTCATAAATTTGAGCAAATTTTTAAA
GTGGCAGTAATCCTTACATTTTGATTCAGTTGATTTTCAGATCCCCTTCA
CATATACGTATTTTACAGTTGCAATTATTTAACATTAAATGTTATACAAT
TGTATATATTGTCTAACTACATATCATTGTTACAATTTTGTCAAAATGTT
ATACAATTGTATATATTGTCAAACTATTGTTACAATTTTGTCAAAATGTT
ATACAATTGTATGTATTGTCTAACTACATATTCTTTGCTACAATTTTGTC
AAAATGTATAGGATTGTATATATTGTCTAACTACATATCGTTACAATTTT
GTCCTTGTTTGTTGAGGCAGGATCACTCTGTGGCCCAGGCTGGAGTATAG
TGGTTTGATCATAGCTCACTGCAGCCTTGAATGCCTGGGCTGGAGTAACC
CTCCCACCTCAGTCTCTGGAGTAGCTGAGACTACAGGCATATGTCACCAC
GCCTGGCTCATTTTTTATTTTTTATTTTGTAGAAATAGGATTTTGCTATA
TTGCTCAGGCTAGTCTTGAACTCCTGCCCACATGCGATCCTCTTGCCTTG
TCCTCCCAAAGTGGTGGATTTATAGGTGTGAGCCACTGTGCCTGGCCTCA
TTGTTCCAATTTTAATGATGACATAGGACTCTTATCGTGATGTATCATGA
CAATTAGACTACTCCCCAGATGTTAGATATGTCAGTTGCCCTCTAACAAA
AAGTGCTTTAATAAATAATTTTGTATATATGTCTACTTTAATAGGAGTGG
AATTATTTGGTCAAAAGAAATGAATCTCTATGGCTGTTCCCACATATGAC
CATTTTGCTCCTTTTAAAGTATACAAACAATCTTACAATGTTAACAGCAA
AGAAGGCATTGTTTAGAAAAATGATATCATTGGGCCAGGTGTGGTGGCTC
ACCCCTGTAATCCCAGTACTTTGGGAGGCTGAGGTGAGAGGATTGCTTGA
GCGCAGGAGTTTGAGATGAGCCTGGGCAACATAGTGAGACTCCATCTCCA
CAAAAAATTTAAAAATTGGCTTGGCGTGGTGGCATGTGGCTGTAGTCCCA
GCTATTCAGGAGTCTGAGGCAGGAAGGTCTCTTGAGCCCAGGAGTTTGAG
GTTACAATGAGCTATGATCATGCCACTGCACTCTAGCCTGGGTTACAGAG
TGAGATTCTGTCTCTTATTAAAAAAACAAAACAAAACAAAACAAAACAAA
AAAAACCCCCGATGTCATTGGGAATTACTTTTAAAAAATAGATTTCCTAG
TTTACTTTTCACTACCATTTACAAGAATGCACATTTCTACATTTTAACTT
AGTTCTTCTTCTGGAAAATGTTTTGTCAATATTTCTGTTGAAGATTGGTA
TTTTTAAAATATCACAGACTCTTGAAGAAGTGTCATATTTCTATTGTTTT
ATTCTCACTTTATACTGTGTAGAGACATAATTTCTTATTTTTCTTTATGA
TTTCTATTTAAAAATCAAGTTAACTTTTCTAGTCTATTTTGATTTTTCCG
TAATTTTAGATTTTATAATTTAACTTTCCCACCTTATTTAACCTTAATTT
ATATATTTATACCTATATCATTTTACAAAGAATTTAAGAAGGTATTTAGC
ATTTTATATAAAATGAAAAAACACACATAATGATGGTCTGAAATATAAGC
ATATTAAACATAATGCTTGGTTTGAGCTTAGATTTGATGCTGGGCTGTCT
GGTTGCTGGTGCAAAAATTAAAAAAACACAGATTGTCAATTATCAGTGAT
TTAATGTTTTTGCAGAACTTTTTTTTTCTACCCAATAAGACAGCACATGA
CTAGATGAACAAGAGAGATGCCTGCCTCTCCCCACCCCTTTCAGTGAGCA
CTGAAATATCTCAGGTGGATCCAGAATTCAGAAAACAGTCCGAAAAAGGA
GTTAAATCATCTCATCATTAGAAGGGAGAGAAATATTCTCAGTTTCCCAG
AAGAAAAAAAAGTTAAAAAGCCTTTCTAGCTCTAATTTGTAACAAAACAA
AACAAAAATAAAAATGAAAACACTCCACATGAAACATTTAGGGCATTTAG
CTGTGAGCTGTTTTTACACACAGCTATTTCTTAGAGCAAACTTTCATAAC
CTTTGAAAGCCCAGTGTCAATGTGCAACTCAGTTAAAGTGCTTCTAAACT
TGTTTAAGGGTGTTGTTTTTGATGGCTTAACTGGTCTATCAGTAGCATTT
AATATTTCTAGAGGTCTAAAAATACATTTTAAAATTCTGTAGAAATGGAT
GTTTTCCTCAGAAAAACATAATAGTCACTCAATAAAACCCCCAAAAGGCA
AAAGTCATGTGCTAAATTGAGAATTCAAGACATTCCTTCTAAATATCTTT
ATTTTTGGTCCTGACTTTTATGGGAAATCTGTCAGACTTTCAAGTAACAG
TTAAGTTCTATGGTATGTAAATTGTTTCAGAGCATAAACTATAAAAAAGT
TCCTAATTCACTTCACAAAGCTATTAACCCTAATACCAAAATTTAAGACT
GCAAAGGCCTTCTAATTGTTCTTTGCTTTTCTGTCTCCACTTCTAATCTA
AGCACTGGCACTAGAGTGCTTTTTAAGGTGCCTATCTTATCACATTACCA
GAAAGGGGTACTTCTCAACCCCTTGAAAGACTGGCAGGAGAAAATGACAG
CCCTATCTGTTGGAAGTGGCAGAAGACAGCATCAGGACTGGCAGAGAAGC
TGGAAATAACCTAGAAAAATCCTGGAAGAAAGAGATCTGCAGAAGGAATA
GAACCCCAAATCTGACTATAAACTCTGCTGTAATCCCTAGCTAACTGCAT
ATGAAGGATAGTGGGAGATGGACACCCTACAGGGGCCATTGAAAAAGCAG
ACAAACCAGCAAAAAGTCAATCCTTGAAAGATAAAGTTGTGCTAAGATTA
ATAGCTTCTTTATATTTACGTATGTATGTATGTATGTATGTATGTATGCA
TGCATGTATTTTGAGTACATTCCCTAACCTCAGAAGTAGGTATCAGAGTA
AAGAGTTTTGGATTGGCCAGGCACGGTGGCTCACTCCTGTAATCCCAGCA
CTTTGGGAGGCCGAGGCAGGTGGATCACTTGAGGTCAGGAGTTCGAGACC
AGCGTGGCCAACATGGTGAAACCCTGTCTCTAATAAAAATACAAAAATTA
GCCAGGTGTGGTGGCACGCGCCTGTAGCCCCAGCTACTCTGGCGGCTGAG
GCAGGAGAATCGCTTGAACCTGGGAGGTGGAGGCTGCAGTGAGCTGAGAT
CGCACCACTGCATTCCAGCCTGGGCAACAAAAGTGAAACTCCATCTCAAA
TAAAGCAAAACAGTTTTGGATTGATAGAGCAGCTGGAGAAATAGGGGAAC
CTCAGGGTGGGTATATCCAAAAATCTCAGTATAAGCTCTCCAGTTCCTTG
GCTGACTGCTAAGCTCCATGGGCACAAAAGGAGACCAGGCTAGAAAAGCA
GCAACTGGAAGTTATAAAAACTGAGCAAAGAGATCAGCTGCTGCATACTG
CTGGAAGATAGAATTTGAAGTTTGAGTACAGGAATGTTTACTGCCAGCTA
GAATACAAAAAACCAGTAATACCCAGAAGAATAAAGCAGAAACTAGAGTT
GGTAATAAACTATCCACAATGTTAAGTTTGTGGCCAAAAATTACGAGACA
TGTAATGAAACATGAAAGTGTGACCCATATTAAAAAAAAAAAAAGTCAGG
CTGTAATAATTTACTCTAACTAGAGCCAGGTGTTGGATTTATTAACTACT
TCAAACCAGCAATTACAAATGTGCTCAGATAAATTAAGGAAAACATGGTC
TCAATTAGCCAACATAAGGAATCTTGAGAAACTGAAACTATAAAAAAGAC
AAATGAAAATTCTAGAGCTAAAAAATGTTATAGAATTTAGAAGATAAGCA
CAAGACTAGAAATAGAAGAATCAGTGAACTTAAGGATACATCAGTAGAAA
TGATCTAATCTGAAGATCCATTAAAAAAAGAAAGAAAATTGAACATGTCC
TCAAAGACTTATGGGATAATACCAAGCTTTTCAACATATGTTTAGTTGGG
GTCCTACAAAGAAAGGAGAGAATGAAGTATTAAAAAAGTATTTAACAAAA
CAATGGTTAATAATACTCCAATTTTGGTGAAAACATTAACCTCTAGATTC
AAGAAATTCAGTGATCCCCAAGGGGGGAAAAACCAGTTAAGATGCATCAT
AACCAAACTACTGACAAGCAGAAAGAACTCTATGAAGGGATCATTTTACT
GTATATGTATTATACTTAAGTTAAAATATTTCAAAGCAAACAAGGGAAGG
GAATAAATATAGGATTAGAAAACAATGAAGTAGAAAAACAGAGTGAAGCA
TGTGAAAAAAAAAGCTAGGATTTTGAAAAGGCCAATAAAACTGAAATCTC
TAGCTAGACTAATCAGAGGGTATAAAAATAAAAATTTTGAATAACAAGAG
TGAAAGAGTAGGTCGGGCGCGGCAGCTCATACCTGTAATCTGAGCCCTTT
GAGAGGACAAGGTGGGCGGATCTTGTGAGGCCAGGAGTTCGAGACCAGCC
TGGCCAACATGGCAAAAGCCCATCTCTACTAAAAATACAAAAAATTAGCT
GGGTATGGTGGTGCGCACCTGTAATCCCAGCTACTTGGGAGGCCGAGGCA
GGAGAATTGCCTGAATCTGGGAGGCAGAAGTTTCAGTAAACCGAGATTGC
ACCACTGCACTCCAGCCTGAGAGACAGAGTGAGACTCCATCTCCGGGGAA
AAAAAAAAAAAAAAAAAAAAAGAAAAGGATCAAAGAGTAAAAGAGTAGAC
ATTATTAGAGATCCTAAGACACTGAAGAATGGCGGAGATTATAAACAACT
TTATGGGAATAAAGTTGAAATTTAATTGAAATAAATGACTTCTAAGATAA
ATTACAAAAACTCAGGAAATAAAACCTGAATAGCCCGATATCTATTAACG
AAATTGAATTTGTAATAAAAAATCTTCAAATAAAGTATAATCTATGCCTT
CATTGGTGAATTCTGACATTAAGAAAAATATCATTTCTATATAAACTCTT
CCAGAAAATAGAAGAATGAACACTTTCCGAATCATTTTATGTAGCCAGCA
TTACTCTGATACCCAAAACTAGACAAAGGCATTATAAGAAAAGCAAACCA
TAGACAAATATCCCCCCCGGAACATAGATGTCAAAAATCCTTAACAAAAT
ATTAATGAATCAAATCCAGTTTTACATACCGAGAATAAACCATGACCAGT
GGTATTTAACCTGGGAATTCAAGGTTATTTTAACTTTGAAAAAGCAAGGT
AATCTACCATAAACAGAATAAAACACCTTATGATCATTTCAAAAGATACA
GAAAAACTATTTGAAAACATGTTATAATACGTTCATGATTAAAAAAACAT
TCAGTAAACCAGGAATAGAAGAGAACTCCTTTAACCTGATAAAAAGATTT
ACAATAAAGAACCTTACAGCTACTATACTTTGTATTGAAAAACAGAATGC
TTTCTCTCTCAGACTGAGGGACAAGAATGTCCACTCTCACCATTTCTATT
TAGCAGCATACTGCGAAGGTCCTAGCCATTGCAATTAGGCCATAAATAAA
ATGAATACAGATTGGAAAGAAAAGAGAAACTATTTACAGATGACATGATA
GCATATTTAGAAAATCCTAGGGATCCTCCAAAAACAGCTATTAGGATTAG
TGAGATTAGCTAAGTCTCAGGATTCAATATCAATATACAAAAAAAATCGA
TTTTATTTCCATATACCAGCAAAGATTCCATTTACATTATTCAAAAATAT
GAAATAATAAATTTAACAAAATATGTATAAAGTCTGTAGAGGAAATACTG
TAATATATTGTGGAGAAAATTAAAGATGACCTAAGAGATATTTCATGTTC
ATGGTATAGAAAACTCTATATTAAGATGTCAAATATCTTTAAGTTGACCT
ATAGGTTTAATGGAAACTCTATATCCCAATTTTTGGAGGAATTGACAAGC
TCATTCTAAAATCTATATAAAACACAAAGGACCTAGATTAAGCAAAAGAA
CTTAGAATAAACAAAGCTAGAAGATTTATGCTACCTGTTTTAAGGACTAC
AATATAGATCAAACAAGATTGTTTTGGGTCTTCTATGTCTTTGGAATTTC
CAAATTAAAAAGACATACATTGAGTATGTAAAGCAACTAGAATTCTCATA
CACTGCTGGTGGATATACAAAATGGTACAACAGTGTGGATATTTCTTACA
CAGATAAACACATACTTACCATGTGATTTAGCAATTGTCCTCTTAGGTAT
TTACTCAAGAGAAATGAAAATATGTTCACACTGAGACTTATACTCAAATG
TTCACAGCAGCTTTATTCATAATAGCAAAAGCTGAAACAACCCAGATGTC
CATTTATTGGTAAAGGGAGAAATTATGGTATATCCACACAACATAATATG
CTACCCAGAAATAAAATGAACCAACTACTGATATGTGCAACATAGGTGAA
TCTCAACATTATACTAAGTGAAAGAAGTCAAACAGCACTGTTCCATTTAC
ATGAAATTCTTACATAGTTAAAACTAACATATTGAGATGGCAAGCTCAGG
ATAGGTTAGGTTGGGAGGAATTAACTGCAAATAATTTTCAGGGTAATAGA
AGTGTTCTGTTATCTTTATTGGGGTGGTGATTATATAGTAGCATCTACTT
GTCAAAACCTATGTAACTGTAAAGTTGGTGCATTTTATGTTAATCATATC
TCAAAGCTGATTTCAAAAACTTCAATGATTTCCTATAGTCATTAGGATAA
ATCCAGTGGCAAAAAGGTTCTATATCATCCAACCCCTACTTACCTCTCTG
GCTTTATCTTATACTGTCTTTGCCTCACTCTATGCCCACCATGTTGGGCT
TCTCATCCATTAGAATACATACGCTGTCTCCAGCTCTTGACTTTTACAAC
TACTCTTTCCTCAAATGCTCTTTCTTTCCTCAACTCTTATTCATCCTTCA
TTTTTTCCTCAAGAAAACCATCCCGCAAATCTTGGACTAGGATAAGACCC
TTGGTATATACTCTAACAGCACTACTTTTTTGCTATACTTTTCATATTAG
CAATTATTTACCTTATCATTGATAATCATGTATAAAGTATGTTTCCACCC
TCAAGATCAGAAATTCTATGAAACCCGGGTTATCTATTTTGTTCACCACT
CTTCCCCCTGAGTCCCTGATATACAGGAAATCCTCAAATATTTGCTGATA
AAGCAAGCAAATTTTAAAAATCTAAAAATTCTAAATAAAAGAGCATATAG
AATTTAGCAATATATTAATAATACATGTTTAAGTAGGGCTTATTGTAAAA
AATAAGGTTTAGTATTTGGAAGTGTATTACTTTATGTAATTCATCAAATA
TATCAAATAAGATAAATTATCTCCAACTACTGCTACTAGCTGTAGTTGAC
TGCTAGCAGCAGTCAACATTATTCTAGAAACATTAGACAATGCAATATGA
CCAGGAAAAAAAAAGACAGTTATATTCAGATTCTGACCATCTATAGAAAA
ACCCAAGATGATTTGCTGAAAAATTTTAGTAAGCAAGTTCAGTAGGTGGT
CAGTTACAAAGTATATGAATACATATTATAAAAACAGTGTATCTAAAACA
ATGCAGTATTGGTGTAACAATAGATAGATCAATGGAATAGATTAGGGAGT
ATAGGAGGAGATAAAATTTTTGCAATATAAGAAATTTCTATGAAACTGAC
CTTTGGAGAAGCATAGATTATTTAAATAATTGTATTAGGACACTGTGGGC
AAATACTTTCCAATATAAAGTAAGAACTGAAATAATATCTAAATCTGTAA
TTCATCTAAAATTTAACAAATGAGACCATAAGAGTTCAGATGAAAATACA
CAAATGCCACTGTGTATATGTCTATATATAATTAATTAATCTGGGAGTCA
AGGAAGACTTTCTAAGCTGGATCTTAACTGCAAAATAATAAAGGAAAAGG
CTAACATGTTTGACTACAAAATTTATATTTTATATTGTAAACAAAATAAA
AATTAAAATATTAAATGAACACTCAAGATGTGATAAAAATGACGATTTTA
AATTTATAAAGATTTTTCAAAAAATCAATAAGATACACTTAAGCAGAAAA
ATGGGTGAAAACTATGAACATATAATCCATGAAGCAAATACAAATAGTAA
ATGAATATGTCAGGATTTCAAATTAACAGTAAGTAAAAATTGAAGTATTT
CAGTCAGATTAAAAATATGTAAACATGACAAACTATACCTAGTGCAGAGA
GAAATGGATACTTAAATACAGATGATAGGCGTGTAAATTGCCTAGCTTTT
CTGGAGGTCAGTGAGGCAGTCTTATAAAATGCCTTAACGTGCACATTTCT
TCTAGAAATCAAGAAAACATTAGACAATTGTACAGTACTCAGTGATTTGT
AATGTGGTTTTGTTTATAATACCGAAAACCAGAAATAATCAGCAATAAGT
GACTGAGGATAATTTGATACATTCATAAAATACAATACCCATACAGACTT
TAAAATGACATTCCATATATATATATGGGCATATATTTGACATTGAAAGA
TGTATACAAGTAGAAAAAAGTATATATATGGAATGATACTCAAATATATA
TATTTAATGACAGGGTCTTACTCTTGTCACCGAGGCTGGAGTGCAGTGGT
GTGATCATAGCTCAGTGCAGCCTCAACCTCCTGGATCCAAGTGATCCTCC
TGCCTCAGCCTCCCAAGTTGCTGGCACTACAGGTGTGGTATCACCACACC
CGGTTAACTAAAAAAAATTTTTTTAGAGACAGGGTCCCACTATGTTGCCC
AGGCTCACACATATATATTCAAAGACATAGAAACAACTTTCTAAGAACAT
TTATTGACATGTTAACAACACTTCTCTCTTGCAGGAATTATACATTTCCC
TCATTTTTGTATTTTTCTGTGTTGAAGGTTCTTTGACAGAACAAGAGCAC
TTTCTAAAAATAGTTCAAACATTGGATGCAAAGGGATGAGCTGCTAGATT
ATTTCCTTATGTGACTATTGTGGTTTTAAAAAGTATAATGGGGACTGGGA
ACACTTTAAGTTTCCAAAGGTTAGATAGGCATTTCTCAAAGTGCATTCCC
ACACCATCCTCTTCAGGATCTGGTATGCTTGCTAAGAGTTTCTCTGTCCC
CACCCTAGCTCTACTGAGCTAAGATCACTGGAGAGGGAACCTAGGACTGG
AATTTTAAACAAACTCCCAAAGAGATTCTCACGCATACCGATATTTGAGA
ACCCTTGTGATACTTCCTATAAAGCTAGAGGGCAGACAGTTGAAGGTTGT
TCTTGCAGAGGGAAAAGGCTGCCCAGTTTGCAAGCAGCAGTTTAGGTGAA
GCTTCCTTTTCCTGTGCTTTAGCAAGCTTGCTTCATCACCTATTTATTAT
GTTTGCACATATAGTTAAATCTATGTCTTGACTCTATCCTTTCTACCAAT
TTGTTTTTTCTTACTTGGGGCCATATTACCAGGGTATATCATTTTATTAT
ATATGATATCTGATAGGATTACCAGATATTATATATAATAGGTATATATA
ATATAGGTATATATATATTATATAGGTGTATATATAATAGGTATATATTA
TATAAAATAGGTGTATTATAGGATATTATATAAGAGGTGTATTTTTCCCA
TGACACTTTTTTTGGCTCCTCCATTTAGCTTTAAGTAGCAAGTGTCATTC
AAATCAGCTTTCTCTTAACCAGAATATTGTGGCCTACTTGGAATCAGAAA
CTTTTCACAGCCAATTATCTAAACCAGTTCTGAAGTAGACTATAACTGGG
AAAATGGCAATGTAGGTTCTGTCTCTTCCAGAACCATCTGCTTCTGCTTA
TGGTGTCTTTTCAGCATCTACAGGGTCAGTCTGTCATGCAAAAGAGCATT
AATTAAGTAGTTCTCTCTCCAGCAGATGCTTGAAGGAAATCCACAACTCA
ATTTCCACAGTAATTGGCTCCCAACACACCTTTTCTTACAGAGAGATAGT
GATTGTTTTCTTTTCTGTGGTTTCCATTTTCCATGGCACCATACTCCTAT
ACCCGCAAGCAAGCCTTACCGGCATGGTGATCTGGATGGGTTGCCGTTCT
CCACTCTTCGTTGGGGTCACACCTTCTGTGTCATATGTACCCGTATACAA
CTCTGTCCCCATCAGGCTCTTTAGACTTCAGTTCCAGTGGGACAAGCACA
CCACCAATGGAAATATTCCTGCAATAGACTTGAGTTGACCACATACGGTT
GAAATTAGCCTAGGCACCCTGTCCTGTTTTGGACCAAACTCTAGGCAGGA
AACTTACAGGGTTTCACTAAGGGGGGCATTGTGGTGCGGGAGAAAGTTCT
GCAAGTTCAGGGGGCAGTGCTTCTCCTATTTATGAATTAAAAACCTACCT
ATCCCCAGCCCAGACATTAGGATTCAGTTGGCCTGGGATGTAGTTTGGAC
ACTGGTACATTTATTTTAAGGTTATCTAGGTGATCTTAATATGCAGGTAG
GATTGAGAATACTGAACTCGTCCTCTAATATTTTTTTTCTCCTGTTCCAA
CAATCAGGGGTCCTATGATTTGGAGTACCTCAAGCCTTCTACACCATCAG
AAATTCTCACTGGCATCATTCTTTTGTGTTAGGATAATGGTTCTCAAAGC
CAAGTCCCTGGACCAGCAGTATCAGCATCACCTGCAGACCTTAGAAAACA
AATTCTTAGGCCCTACCATAGACTAAACTAGAACTTCTAGGGAGAGCTCC
AGCATCTGTGTTTTAACAAGCACTCCAGGTGATTCTGATATATACTCAGG
TTTGAGAACCACAGGATTAGAGTTGAGAAATCCTTGATAGCATTAAAATG
TAACCAGAAAAAATAGTTTTATTCCTAACAAATTAACTTTTAGATTAGTT
TGAACACTAGAGGAATACTCTAAAAAAAAAAAAAAGAAATATATTAACAG
ATAACTCCGGGGATAAGGTTGGCAGGAAAGAAGGGAAAGGAAAAGCCTGG
ATTATATCCAGGTGCACAGAACACTGGGAAATGTGAGAATAAAGGGTGCA
TGGAGCTGTGGGGGATGCTGACAAATAGATGAATCAGGAGTACCAGAAAC
CATCCAGATTTGGTTCTAGGTCCTTGCACCTTGGAAGGCTAATACAAGTT
TACTGAATTAAAACGTAAGCTATAAGTAGTATCTGATGAATTCAAAGTCC
TTCCACCCCCGCAATAATGCCCACCATATGTAGTAGATACAATAAATGCT
GGTTTATTTTCTCTCTGCAGACTAGACTTTGGTAGGTAGAAACGAGAGGT
AAAGGGGCTCCACCCGCAGTTCTTCCGGGTAGGCTACTGCATGCTCCCAC
ACACTTTCCCGCCAGAGGTCTACTGGGTGCATTATCCAGCGGTCGCTGGC
AAGAAATCGTCCCTGCCATGGATAAACCAAAAAACATCCTCTTGCTGTGG
GGATGTTGCTAATCCCAGGCACCATCACCAGCCTGAACAGAAAACTCAGG
AGAAAAAGAACAGCATACGGCATGGAAAACCGTGAAACTAGGCCAGAGAA
GGGGCGGCCGGTCTCTTACTAGTGTCTGCTGCTCCACCCCAGGGTCCCAG
CCACTGAATGGCGAAGGGAGTGGGGAGCATCCCTCAGGGAGCCCCAGTAA
TCACCCCTCCCCTGCCTTTCCACCTCATTCCTCCTTTCTCCCTCCTTCAG
CCTTGCGGGCAGACCCTGTGGGCCGCCTGGACCGCGCGCAGGAGGGCTGG
GATTGCGGTGGCTGAACCCTGCGGACCTCTCCCATCTGCTCCACCCCGAC
CGCCTGCGGTTCCGCGCCCAAGGCTGGACAGAAGGCAGGAGAAATTTATA
AGAAACAGACAAGCAAAAACCCTGGCTTCTTGTCACTGATTTTAAAGAAC
CCACTGAGGTCACTGCGATGGGTGGAGGGAAGCGAGAATGGAGGAATACA
AGCCAAAGGGAAGGAAGGGGACGAAGGCGGACAGGGAGTGACCTCTTCCT
CCAACCCCCGGGCCCGCTGGGAGCGGCGCGAGGCCAGAGGCCCTTGAGAG
GCTCGGGCTGTCCTGGGGGCCTCAGTCCTCTGCCTGTACCCCATGGGGGA
CCCTGCTGCCACCAGGCGCCCCGCACTCACTCGACCTGCAGCGTGCTGGG
TTTAATCTTCACCTCAACCTTGTAGGAGGAGCCGGTGAGCAGCTTGATGG
TGCGGTTCTGGCCGAAGCGCTGCCCGTCCACCTTGTAAAAGACCGGGCCG
TCATTAGGCTGGATGCGCAGCGCGATGGAGAGGCGCACGAGGCCCGGCAG
GTCCCCCATGTCTGGGCGAGGGTCTGGCGCGGCGGCTCCGGGGGGCGGAG
GACAGCGCCGGCTGCGGCCGAGTGGCTGGAGCGCGAGGGGCGGAGAGGAA
GCGCGGGGAGGGTGAGGGAGGTGGTGGAGCTGAGGCTGCCGCTAGGAACC
CGCGCCGTCGCCGCCGTCCGCCCGGGCTTTTGAGGAGCAGCTCCTTAGGC
TGTGGCCCCCCTCCCCACTCGGCGAGGAAGCGGGCCCAAGAGACGGCTCC
AAGGCCGCGCGCTTCCCCATCCCCCGCTCCAGTGCTGCGCCCTCCACGCA
CCCGAAGGCTCGCTCTGGCCCGCAGGCCGCCGCGCAGATCCGCGCAGCTG
GGGGCGAGGGAGTTAATCCTGTTTACGCACCACAATCCCCTTCAGCTGGG
GAAGCGGACATTTAGGCTCCTCCTAGAACAGCCCCGGGCAGGAGGAGGAG
AGGTTTGGGAGGCACTGGGAAGGCGCTGGAGTTAAGCGACCACTATGCCA
AGGAGCGAGACCCCCGGAATCTGGATACCGCCTCGGCCAGCTACGTGAGG
TGGACACTGCTGCTCGCGGATCCGGCGCCAGCCA

cDNA Sequence of CRIP1a from Homo sapiens (SEQ ID NO:4)

ATGGGGGACCTGCCGGGCCTCGTGCGCCTCTCCATCGCGCTGCGCATCCA
GCCTAATGACGGCCCGGTCTTTTACAAGGTGGACGGGCAGCGCTTCGGCC
AGAACCGCACCATCAAGCTGCTCACCGGCTCCTCCTACAAGGTTGAGGTG
AAGATTAAACCCAGCACGCTGCAGGTCGAGAATATTTCCATTGGTGGTGT
GCTTGTCCCACTGGAACTGAAGTCTAAAGAGCCTGATGGGGACAGAGTTG
TTTATACGGGTACATATGACACAGAAGGTGTGACCCCAACGAAGAGTGGA
GAACGGCAACCCATCCAGATCACCATGCCGTTCACAGACATTGGGACCTT
CGAGACAGTGTGGCAAGTCAAGTTCTACAATTACCAGAAGCGGGATCACT
GCCAGTGGGGAAGCCCCTTCTCTGTCATTGAGTATGAATGCAAGCCCAAC
GAGACACGCAGTCTGATGTGGGTGAACAAGGAGTCCTTCCTCTGA

Deduced Amino Acid Sequence of CRIP1a from Homo sapiens (SEQ ID NO:5)

MGDLPGLVRLSIALRIQPNDGPVFYKVDGQRFGQNRTIKLLTGSSYKVEV
KIKPSTLQVENISIGGVLVPLELKSKEPDGDRVVYTGTYDTEGVTPTKSG
ERQPIQITMPFTDIGTFETVWQVKFYNYHKRDHCQWGSPFSVIEYECKPN
ETRSLMWVNKESFL

cDNA Sequence of CRIP1b from Rat (SEQ ID NO:6)

ATGGGGGATCTACCGGGGATCGTGCGCCTTTCCATCGCGCTGCGCATCCA
ACCCAATGATGGCCCTGTTTTCTTCAAAGTGGACGGGCAGCGCTTTGGGC
AGAACCGTACAATCAAGCTGCTCACCGGTTCCTCCTACAAGGTTGAGGTG
AAGATTAAGCCCAGCACGCTGCAGGTCGAGAATATTTCCATTGGTGGTGT
GCTTGTCCCACTGGAACTGAAGTCTAAAGAGCCTGATGGGGACAGAGTTG
TCTACACCGGCATATACGATACAGAAGGGGTAGCCCCAACCAAAAGTGGA
GAGCGACAACCCATCCAGATCACCATGCCGTTCACTGACATTGGGACCTT
CGAGACGGTGTGGCAAGTCAAGTTCTACAATTACCACAAGCGAGACCACT
GCCAGTGGGGAAGCCCGTTCTCCGTCATTGAGTATGAATGCAAGCCCAAT
GGGACGCGCAGCCTCATGTGGGTGAATAAGGAGTCCTTCCTCTGAAAATG
GCTCCTCCTGACAGTTGCTAGACAATCTCCGGAAATCGATGCTTAGACAA
ACCAGCACAAGCCTTAACCAAGGCACACCATACCATGGCTGTTTCCCAAC
GACCTCCTATCCCCAATTACTGTGAAAGTGTGATTCCCTCCTGAGGCAGT
ACCTGTGACATCGCGTGGGATGTCACGTCCCTGAAAGTTCTCCTTCCACT
ATTGTATACTGGTGTTCCCGCATCTCTCTTGTCTGCCAGTGTGAATGGAG
CTCAGTGATAGTATCAGGTTTGTCCTATACCAGAAAGTTCCACAGAAGGA
CCATAGTGTTTTGTTTCCGGGACAAGAAATTTCTCCCTTTTGTGTTCATT
GTCAAATTAAGATAACAGTGTGCTGGAGTCCCCCATGTGTGATTTGTAAC
ATGTAGCATGCAATGTTTGTGAGCACCCCAAGAAGAGGTAATAACATAAA
CAGCGGTTAACGGGACCCAAATGTTGTGCTTCCCATCAGCAGCTGTCAGC
TTCTTCAGAGGAAGAAAGATGATGAAGAAATAAAAGAGATGAGTATATAA
ATAAGAGTTTATAATGTATCAATGGTNTTCCAGGNAGCGGGGTATTTTGC
TGTGATNTAGTAGAAACAGGGGAAAGACACTGTTGTAGAATCGGAGCTGT
GGCTCGCCCCTTTGACAAACTCCCATAGACTTTAAACCAACATAGAGATG
GCGCCCCTGCACACCCTTTCACTTGAACTTTGTACAACTGTGTATGGAAT
GGGTAATCAACTTTTGATATTTCTTAATAAAGACATTGAAAAAAA

Deduced Amino Acid Sequence of CRIP1b from Rat (SEQ ID NO:7)

MGDLPGIVRLSIALRIQPNDGPVFFKVDGQRFGQNRTIKLLTGSSYKVEV
KIKPSTLQVENISIGGVLVPLELKSKEPDGDRVVYTGIYDTEGVAPTKSG
ERQPIQITMPFTDIGTFETVWQVKFYNYHKRDHCQWGSPFSVIEYECKPN
GTRSLMWVNKESFL

1. An isolated nucleic acid, wherein the nucleic acid comprises a polynucleotide selected from the group consisting of:
a) a polynucleotide as defined in SEQ ID NO:1, SEQ ID NO:3, or SEQ ID NO:6;
b) a polynucleotide encoding a polypeptide as defined in SEQ ID NO:2 or SEQ ID NO:7; and
c) a polynucleotide complementary to a full-length polynucleotide of either a) or b) above.

2. The isolated nucleic acid of claim 1, wherein the nucleic acid comprises a polynucleotide as defined in SEQ ID NO:1.

3. The isolated nucleic acid of claim 1, wherein the nucleic acid comprises a polynucleotide as defined in SEQ ID NO:3.

4. The isolated nucleic acid of claim 1, wherein the nucleic acid comprises a polynucleotide as defined in SEQ ID NO:6.

5. The isolated nucleic acid of claim 1, wherein the nucleic acid comprises a polynucleotide encoding a polypeptide as defined in SEQ ID NO:2.

6. The isolated nucleic acid of claim 1, wherein the nucleic acid comprises a polynucleotide encoding a polypeptide as defined in SEQ ID NO:7.

7. An isolated nucleic acid, wherein the nucleic acid comprises a polynucleotide encoding a polypeptide having at least 80% sequence identity with a polypeptide as defined in SEQ ID NO:2 or SEQ ID NO:7, and wherein the nucleic acid encodes a polypeptide that binds a CB1 cannabinoid receptor protein.

8. An isolated nucleic acid, wherein the nucleic acid comprises a polynucleotide encoding a polypeptide having at least 80% sequence identity with a polypeptide as defined by positions 1 to 60 of SEQ ID NO:2 or SEQ ID NO:7, and wherein the nucleic acid encodes a polypeptide that binds a CB1 cannabinoid receptor protein.

9. An isolated nucleic acid, wherein the nucleic acid comprises a polynucleotide encoding a polypeptide having at least 80% sequence identity with a polypeptide as defined by positions 61 to 110 of SEQ ID NO:2 or SEQ ID NO:7, and wherein the nucleic acid encodes a polypeptide that binds a CB1 cannabinoid receptor protein.

10. An isolated nucleic acid, wherein the nucleic acid comprises a polynucleotide that hybridizes under stringent conditions to a second nucleic acid selected from the group consisting of:
a) a nucleic acid comprising a polynucleotide of SEQ ID NO:1, SEQ ID NO:3, or SEQ ID NO:6; and
b) a nucleic acid comprising a polynucleotide that encodes a polypeptide of SEQ ID NO:2 or SEQ ID NO:7.
wherein the nucleic acid encodes a polypeptide that binds a CB1 cannabinoid receptor protein, and wherein the stringent conditions comprise a hybridization in a 6× sodium chloride/sodium citrate (6×SSC) solution at 65° C.

11. A vector comprising the nucleic acid of claim 1.

12. A vector comprising the nucleic acid of claim 7.

13. A vector comprising the nucleic acid of claim 8.

14. A vector comprising the nucleic acid of claim 9.

15. A vector comprising the nucleic acid of claim 10.

16. A composition, wherein the composition comprises the isolated nucleic acid of claim 1 and a pharmaceutically acceptable carrier.

17. The composition of claim 16, wherein the isolated nucleic acid comprises a polynucleotide as defined in SEQ ID NO:1, SEQ ID NO:4, or SEQ ID NO:6.

18. The composition of claim 16, wherein the isolated nucleic acid comprises a polynucleotide encoding a polypeptide as defined in SEQ ID NO:2, SEQ ID NO:5, or SEQ ID NO:7.

19. A method of modulating the activity of a CB1 cannabinoid receptor, comprising administering an effective amount of a composition to the CB1 cannabinoid receptor, wherein the composition comprises a polynucleotide selected from the group consisting of:
a) a polynucleotide as defined in SEQ ID NO:1, SEQ ID NO:4, or SEQ ID NO:6;
b) a polynucleotide encoding a polypeptide as defined in SEQ ID NO:2, SEQ ID NO:5, or SEQ ID NO:7; and
c) a polynucleotide complementary to a full-length polynucleotide of either a) or b) above.

20. The method of claim 19, wherein the composition further comprises a pharmaceutically acceptable carrier.

21. The method of claim 19, wherein CB1-mediated tonic inhibition of Ca²⁺ channels is decreased.

22. The method of claim 19, wherein CB1-mediated sequestering of G-proteins is decreased.

23. The method of claim 19, wherein the CB1 cannabinoid receptor is in a cell.

24. The method of claim 23, wherein the cell is a human cell.

25. The method of claim 24, wherein the cell is in a patient.

26. The method of claim 25, wherein modulating the activity of the CB1 cannabinoid receptor in the patient results in one or more effects selected from the group consisting of appetite stimulation, analgesia, euphoria, decreased tremor or spasticity associated with multiple sclerosis, attenuation of nausea and vomiting in cancer chemotherapy, reduction of intraocular pressure, decreased intestinal motility, and attenuation of aversive memories.

27. The method of claim 25, wherein modulating the activity of the CB1 cannabinoid receptor in the patient results in one or more effects selected from the group consisting of appetite suppression, decreased sedation, decreased alterations in cognition and memory, and decreased mood alterations.

28. A composition comprising an isolated polypeptide, wherein the polypeptide comprises a polypeptide selected from the group consisting of the polypeptides as defined in SEQ ID NO:2, SEQ ID NO:5, and SEQ ID NO:7.

29. A composition comprising an isolated polypeptide, wherein the polypeptide comprises a polypeptide having at least 80% sequence identity with a polypeptide as defined in SEQ ID NO:2, SEQ ID NO:5, or SEQ ID NO:7.

30. The composition of claim 29, wherein the polypeptide comprises a polypeptide having at least 80% sequence identity with amino acids at positions 1 to 60 of a polypeptide as defined in SEQ ID NO:2, SEQ ID NO:5, or SEQ ID NO:7.

31. The composition of claim 29, wherein the polypeptide comprises a polypeptide having at least 80% sequence identity with amino acids at positions 61 to 110 of a polypeptide as defined in SEQ ID NO:2, SEQ ID NO:5, or SEQ ID NO:7.

32. The composition of claim 28, wherein the composition further comprises a pharmaceutically acceptable carrier.

33. A method of modulating the tonic activity of the CB1 cannabinoid receptor, comprising administering an effective amount of the composition of claim 28 to the CB1 cannabinoid receptor.

34. The method of claim 33, wherein the composition further comprises a pharmaceutically acceptable carrier.

35. The method of claim 33, wherein CB1-mediated tonic inhibition of Ca²⁺ channels is decreased.

36. The method of claim 33, wherein CB1-mediated sequestering of G-proteins is decreased.

37. The method of claim 33, wherein the CB1 cannabinoid receptor is in a cell.

38. The method of claim 37, wherein the cell is a human cell.

39. The method of claim 38, wherein the cell is in a patient.

40. The method of claim 39, wherein modulating the activity of the CB1 cannabinoid receptor in the patient results in one or more effects selected from the group consisting of appetite stimulation, analgesia, euphoria, decreased tremor or spasticity associated with multiple sclerosis, attenuation of nausea and vomiting in cancer chemotherapy, reduction of intraocular pressure, decreased intestinal motility, and attenuation of aversive memories.

41. The method of claim 39, wherein modulating the activity of the CB1 cannabinoid receptor in the patient results in one or more effects selected from the group consisting of appetite suppression, decreased sedation, decreased alterations in cognition and memory, and decreased mood alterations.

42. An antibody that specifically binds a polypeptide selected from the group consisting of the polypeptides as defined in SEQ ID NO:2, SEQ ID NO:5, and SEQ ID NO:7.

43. A composition, wherein the composition comprises the antibody of claim 42 and a pharmaceutically acceptable carrier.

44. A method of modulating the tonic activity of the CB1 cannabinoid receptor, comprising administering an effective amount of a composition to the CB1 cannabinoid receptor, wherein the composition comprises the antibody of claim 42.

45. The method of claim 44, wherein the composition further comprises a pharmaceutically acceptable carrier.

46. The method of claim 44, wherein CB1-mediated tonic inhibition of Ca²⁺ channels is increased.

47. The method of claim 44, wherein CB1-mediated sequestering of G-proteins is increased.

48. The method of claim 44, wherein the CB1 cannabinoid receptor is in a cell.

49. The method of claim 48, wherein the cell is a human cell.

50. The method of claim 49, wherein the cell is in a patient.

51. The method of claim 50, wherein modulating the activity of the CB1 cannabinoid receptor in the patient results in one or more effects selected from the group consisting of appetite stimulation, analgesia, euphoria, decreased tremor or spasticity associated with multiple sclerosis, attenuation of nausea and vomiting in cancer chemotherapy, reduction of intraocular pressure, decreased intestinal motility, and attenuation of aversive memories.

52. The method of claim 50, wherein modulating the activity of the CB1 cannabinoid receptor in the patient results in one or more effects selected from the group consisting of appetite suppression, decreased sedation, decreased alterations in cognition and memory, and decreased mood alterations.

53. A method of screening for a modulator of CB1 Receptor Interacting Protein 1 (CRIP1) activity on a CB1 cannabinoid receptor, comprising a) combining a CB1 cannabinoid receptor and a CRIP1 polypeptide in the presence and absence of a putative modulator, and
b) comparing the interaction between the CB1 cannabinoid receptor and the CRIP1 polypeptide in the presence and absence of the putative modulator,
wherein the CRIP1 polypeptide comprises a polypeptide as defined in SEQ ID NO:2, SEQ ID NO:5, or SEQ ID NO:7.

54. The method of claim 53, wherein the modulator inhibits CRIP1 activity on the CB1 cannabinoid receptor.

55. The method of claim 53, wherein the modulator increases CRIP1 activity on the CB1 cannabinoid receptor.

56. The method of claim 53, wherein the method comprises an affinity chromatography or immunoprecipitation assay.


SEQUENCE LISTING (16 sequences)

SEQ ID NO: 1 - DNA, 973 bp, Homo sapiens
ggccagagcgagccttcgggtgcgtggagggcgcagcactggagcgggggatggggaagcgcgcggccttggagccgtctcttgggcccgcttcctcgccgagtggggaggggggccacagcctaaggagctgctcctcaaaagcccgggcggacggcggcgacggcgcgggttcctagcggcagcctcagctccaccacctccctcaccctccccgcgcttcctctccgcccctcgcgctccagccactcggccgcagccggcgctgtcctccgccccccggagccgccgcgccagaccctcgcccagacatgggggacctgccgggcctcgtgcgcctctccatcgcgctgcgcatccagcctaatgacggcccggtcttttacaaggtggacgggcagcgcttcggccagaaccgcaccatcaagctgctcaccggctcctcctacaaggttgaggtgaagattaagcccagcacgctgcaggtcgagaatatttccattggtggtgtgcttgtcccactggaactgaagtctaaagagcctgatggggacagagttgtttatacgggtacatatgacacagaaggtgtgaccccaacgaagagtggagaacggcaacccatccagatcaccatgccggagtgcctggaacaacgaccccaagagatctctcttacatatgaatgtgaagaataagcacattcttcaatgtgtgccatcatgaggaacaaatgttcttttttttttttccaaattatttgctttgcattctaggggagggcttctcacaggccatcagaattatcggggatgcttgttaaaatgtagattcctaaactctacctttttagacctcttaaattgagcaaactctctgagagtaaatccaaagcatctgcattttattaagcactttaggtgattcttatcatattaaagtttgggaaaacactaaaaaaaaaaaaaaaaaa

SEQ ID NO: 2 - protein, 128 aa, Homo sapiens
MGDLPGLVRLSIALRTQPNDGPVFYKVDGQRFGQNRTIKLLTGSSYKVEVKIKPSTLQVENISIGGVLVPLELKSKEPDGDRVVYTGTYDTEGVTPTKSGERQPIQITMPECLEQRPQEISLTYECEE

SEQ ID NO: 3 - DNA, 35804 bp, Homo sapiens
agtgttttccaaactttaatatgataagaatcacctaaagtgcttaataaaatgcagatgctttggatttactctcagagagtttgctcaatttaagaggtctaaaaaggtagagtttaggaatctacattttaacaagcatccccgataattctgatggcctgtgagaagccctcccctagaatgcaaagcaaataatttggaaaaaaaaaaaagaacatttgttcctcatgatggcacacattgaagaatgtgcttattcttcacattcatatgtaagagagatctcttggggtcgttgttccaggcactcctgaaaataaatagataccagaataagtttgagaaaaccacagttgggtaggtgcagtgactcacgcccgtaatcccagcactttgggaggccaaggcaggcagatctcttgaggtcaggagttcgagaccagctgaccaatatggtgaagccccatctctactaaaaatacaaaaattagctggctgtggtagtggtgcacctgtagtcccagctacttgggaggctgaggcagagaatcacttgaagctgggaggcagaggttgcagtgagctgagattgtgccactgcactccagcctgagtgacagagcgagactctgtctcaaaaaaaaaaaaaaaaaagaaaagaaaaccacagcgaagcgaattctgtgaagactataaagaagcatcaagctctgtgtaaagggctagattgtttctttgtaaggacaattgtcatcaaatcccatatgtttgttttgttaaaatagccactatttagccaggcccaagctgttttcactaatacactgcatttctgtcaagcttctctttggatgttctccaagcattttagcagctttaagcctgtcttcccttgttccctgagggaggaacaagcacaagttcacgccaactcattagagagacattgctcaagagcagctcctggggcaccagatgcaggcaggagcgagggagtgggatcccttccaggcccctcccctctgcgctgtctcattttccccacccaccctcccaaatgctcattattgtcttcagcaggtggcttccacccctgccttcaagtctgctcagacaatctccctcccaagatagacaagctccaaaaagattcacaggttctacacaatgcagaactgccaaggaccacattggccaaagcaatgtcttcatacaatcaatcttaagggattttgcagagacaatggtaaatgtggattaggcttttgatcagaaggcagaatattcgtgaccagcctgggcaacatggtgaaacccatctctacaaaaattagctggatgtggtggtggccgcctgtggtcccaaatacttgggaggctgaggtgagaggatcacttgagactgggaggttgaggctgcagtgagccaacatcgcaccactgcactccagcatgggctgttacagagcaagaccctgtctcaaaaaaaaaaaaaaaagaaaagaaaagaaaagaaaagaaagaaaggcagaacctttaggcagcagatgtccaccagaggtatgtgtgcatgtgtgtgcatgtgcacgtgtgtgtacagccatatgggttttaatacatgtatagattcattcaaccactgccgctatcaggatacagaacagttccatcgccccagaaagctccctcctgctacctttttgagtgtatccaagctttattttaaggctttaatcttataaaaatctccagggaaaggtgaaatgctatagaaaagttaaagatagtctgtgttcaattatttgaagaaacttgtcattcagggttagattgtttctatagggtgaggacaaattcccattacctatataggtacttacgttgtcttttcaggcaaatttggttagctatcccctcctaccctgtcaagcagtagctatctagcccacaataaactctctgagtggaagcctgatgatttaaagttatgtctaccatacccattatttttcgactaacaataggaatccaaaatcattagcttgtagccatgaaaattctgaaaatagactctcagtgtggccatatgagttacatttcaaagtgtaaccatctagatttggggaaattaaggaccttacgaacacacacacacacacacacacacacactcatatacacattatatagatctacttcacaccaaacacaagcataaaaaataaactgggaaaagatcaagattccaactgggttttgagttggaaaaaacattagtattttaaaatatatgttttgtcaaagaaagtgactatctgataaaggtaaaggaaaaatcagctgtttcagttttgggaaattcacaatgctcctttgtttagtgggcttgttgaacttttctgaacattcaaaaaatatttttgtcaaataatttggacatttcaaattctgcttcaagcatgaagggaggttgccactaacaccattgttacctcttgggccacggtctcccttgggatggtcatttcagagggaggaaatacccttggctgtcagaggccaagggtctataaacttctagatgagggtaagcgtgttgaagtcatactgatttggattttcattctgtctcctctgctcacaatagctatatgaccttgggcaggtagcctaacctctctgagcttaatagcctcattttaaaaatggaaatcatatgtactcttcaggatgattttgaaaattaaatgagatcatatatataaaatgtctgccatacaaaagatagtcaatgaatgctaattttgtctttcctttcctcatttaattcatctctctacctccaaatacagccttatcaaaactaggtcagtcataatttatcttattttttaagaccatggaaggaaaaaaagatggtttaatccttctcctaatccatattgtgacttctaactttctctggcaggaaatgatttcttagaaccaacttcagaccattacttttttctttttggagaagttttactttactgtcttcagcagaaaaggagagtaggtggtctggattccccagatgtggatcaggaaaggcaaggttatgctgaagtaatgaacaaccccagaatcttagtgatggtttatagaatagtttcttgttcatctctccaccccaaaaaagcagattgatacaacataaaggacagataagggataaagcaaatataccaaaatgttaactgtgaaattcaggtgatagatatgggaatgtgtgttatgcaagtctttcaacttttctatacatttcaacatttttcacgataaaatattgggagaaaagtttatttctcacttatgctatgggtccaacaaaaggtggctagggttccatgccgtatcttctcactccaggacctaaggtgatcaatcatcttgaaagtggctatttgccatggcaaagggaaagagagttctgttggcattgcacagactgttggataatttcagcctgaaatgacatgtgtcacttctgcttacaactcattggccagagctaccagcacatggccttagcccaggccatggaggtcagatcctatcatgtgcctcgacgggggagggctggaaacatttggtaagcagcacaagtaattaccctgatgaaaataaacaacttcataaacttcaaagaataacataccttaggactatcattaataatacgaacgtgacaaggcaaattcactggaagcaatgtcagtaaaagaattcagaaacttttttctttctcatccactttccttcttttagtctcctttaaagggagcaggagaactaaccccgtaaaggctaacagattgcaaacttcttgctggctggtttgcagccggccggccagccagccagccagccagccagctctttctagaatctcatcacctttttgttgagtcagctcattttccctgagtcagtaagaagatgtggaagctgtccagagatcttaagaaaagcagaagttaggttgagggtggtaaagcttttcagaaataggataataaaatatctgagtaaacaatatttcaatatttttcttgactcaacgttttagaggagtcaggaaagacttcatagtgacatttaagctggcacttgaaggcgtttgcaaggtaggaaatggagtagggcgttccaagcagagggaacaggctgtgcagtggcaaggaaactgctttggacttggtagatctgcaggagaataagaaattcactgtgaccagagactagaatgcctgtcaggattactgttaaagtactagcgtagggccaaaaatactgtatttttcatttgttagcaagtgggaaatgaaataccctacttgttaaggaaaaattttagtcttacctagtgctctaatagtagttaacatggcttcaaagagaaaagattatatgcaagtaatttgctataaagagaactataggcaaatgagtttctagcaactgaatacttctgtttgttggcaacaattgttggcaacaattaatatatccacagaaactttctttgggaaaacaagttacctactaggctgcaaacaaattaaggcaaaactaccaggttataaaaacaaacactgcaggagagtgggcaggtaatgaggtgattttaaatgaaatacatttgtcaagagaacagtgatggggagaagaaaaacaaggagctttacgtatgatatatcatttaatcctaacacatgttgtgaagtgaatgttattatcaccatttcataaatgtgaaaactaaagatcaaataaagtagcttcttataaaatgagctgtttaaatttgagccttatttttgaatctctgagacatttttcctctgtaaaatggagacatgacacatcctgcctacctcacagggtggttaagaggattaaaagaaataacaaaagaaattgacatcattcttgaccttgactttttaatcagaaaatcttaagttttctgattctgtcttgatgtttgtatattcttcaaaactatgtctttatataggaaacttaagatggctgtttgtggaaaaagtctttctagcatatcttaaaatatgctattttaaactatttttaaataattaaaaattttacctttccaagcatcaaaatagagtataacgtttggccattatctgaaatccattcatggaatattttaggaaaataagttttattttttaaactcctttagtttttctcttggcattttaaataattttgtttgaaaatgtatcgctccttttctttttttctttttttttgagacagagtcttgctgtgtcacccaggctggagtgtagtggcaggatctcagctcactgcaacctccacttccagggctcaagagatccttccacctcagtctcctgagtagctgggaccacagttgcacaccaccacgcccagctaatttttgtgtttttcatagagacatggtttggccatgttgcctaggctggtctcaaactcctgggctcaagcgagccacctgcattggccccccaaagtgctgggattacagacatgagccaacgtgcccaggctccctttcttttttaatgaaatataccaaataatgatattatgtgccaggaactgttctgaaggtttaatgtacattaacttatttaatcctcataaccattctgcaaggtaaatactattatctccattttccaaatgagaaaattgaggcacagaggtattaagtgacttgcctaagaccacacagctaataaacagctgagctgggatttaaattgaggtatttttgcccagaatctgaactttttttttttttttttttttttgagaaggagtctcctctgaagcccaggctggagtgcagtggcatgatcccggctcactgcaacctcctcctcctgggttcaagtgattctccttcctcagcctcccaagtagctgggattacaggcgtgcaccaccatgctgggtaaattttacatttttagtagagacggggttttgccatgttggccaggctggtcttgaactcctgacctgaagtgatctactcgcttcagcttcccaaagtgctgggattacaggcgtgagccactgcgcttggtggagaatctgagcttttaaacactgaaccatgatacattactgatgataatgggccctctttctaaatgaatcaggaatatcattaggcttatcaatttttatttgatatatacagaaactactgtggtttaatacaggattatttatgtacctacaggatacagttgtccccatctactgttcttttgatttactttgtctgctttttatagttttctctgtcctctctgaaaaaaaaagaatattttcagtatatctgcctctacaaattgtcttcttccctttcatgttctacttccaatttcataagagtcagaaaccagatcaccccacatgttagaattgtgtctaaaacaggagtcaagaggccttgggagaagacccgagcctttagtgaatggacttggtgacatctgtgacagggatctttattttttggcatgttttctgttcttaactgtgatccagatagtttaaatggctggataagtgacttctcagtaagcgaggtgtcacaaaatataaactcaaggcatttctattcttccacctgttcaaaagtgttttagtcatcatcataattggaaggatggcctagttcctcaagcttctgggtagccatctgatgcttggaggtagagagacgtggctacaacttgtataaaaatagcccttccagatggctgaggggcaaagttacaactcagcagctcgatatagctgagcactcaaattttggagtaggaggagagctatgtgagaagtagccagagagactgggctggagcccaatcataagtagaacagtatatgccaggagcgatcacttaacgtgcagattagctgttaagtttttctgttacttcatctccatctcctccccaactcccttgattcaaccttacttctggccctaaggaaacacatacacatacgacatagacagatatgcacacacactttccccttttttttttaaaggagtataaaatagaggaacaaaccataactcttaaaaggcttcagaattctactagtctaacccttcattttagagataagcaaactgaggcagagaaaggtgtggtgaccatccaggatgtgagcaggactcccacgctccaggtgctctgccagggctcatcttctggctcgtttgtgggacactgataacagcgcagctatcccaggacatgtttcctccaagtctgctttgactcatttcaaactctaaataaagcctttagttaaaaaagaaaaaaaaaagtatgggcccagcgtagtggctcacgcctgtaatcccagccctttgggaggccaaggcaggtagatcacgaggtcaggagtttgagaacagcttgaccaacatggtgaaaccccatctctactaaaaatataaaaattagctgagcgtggtagcacacgcccgtaatcccagctactcaggaggctgaggcaggagaattgcttgaacctgggaggtggaggttgcagtgagacaagatcatgccactgcactccagtctgggtgacagagtgagactccatctcaaaaaaaaaaaaaaaaaaaaagagtggattattgcaaaagtcatgataatgtctccaccctcaacacaaatactcaagctctagtccctggcttgaaaacctgtttcaccagtgaaaatatttactaatcaaattagtaaatattaaattgtgtagttcccactttttattttaaatcttttattcactctgcatttctattggatattctggaagaaaaacctctgtagtaaatccattgttttaaatatctcaatgtctgttcaaataattgagtgttggttttaaaaaatagtcattaggaatgggttagtacttacgttttagcaattgtaatgaagactttaaaaaaaaacaaaaaaacaaacaaaaaacacaagcttattgagaaacatgttacactcctttaacatcatttatgatccccaaaggcaaggcattgctgttccctggtggctgtacagcatcactgcaggtaagcgatttggaggtagaaaaggtgttgagtgccagcctttcaaattcagttctacaatgaatcaacttttaaaaatactcccagtagcttgcattttcttataagaagggtttacaggtagcaaaaacaaacaatccccaacgaactttttccaatactataggatttgtgctgcccaagtaaatattacattattggaaaatagcatgttacttttcaagaggaagttcacttccttcttatggaatttatccttaaactgtatggtatatgggctttgtgcaaggtgctggaagtaggcaaggaggcctcaaccatgatgtacacacactattatagcaccattgctctcctgctactgagtcacatagctcaccaactggggaggggtcaccacagaaagagtaatggagcgagataggtacaagcattttttgattgcccgccaagcacctgggcctgtgctagccctgttaaacttggctggagcaatcctgctaaaaggtcaggcttgattcatgaataggtgtgctaattctactccggatacatccccgtcatcctgtagcatacaggcctgagtgaagctggcctagagcagggattcacctgctagataattattgcccaaattaagaatgcaagtcagggcatgattttcaacgcctttattaagaaatatcaaaagttgattacaggtccatatgcagttttacaaagttcaagtgaagaagactgtagggatgccatcaatgtgcgtgtctgaagactatggaagcttgtcaaaggggtaaccctacaactcctgtcactttaacaatggtccacagcaatgcttttcccccatttctactaggctaggccattgcacaataccttaagctacttaaaagagttttaatacgttataaatacgtacatatttgtccttctagtttgttaccatccttccctgaaagagcggagctgtttataggaagcacaacatttgagtcccattcactgctgtttgtattacattttcacaaagcctgctttgaaagctggcaaacactgcaagttacatgttaccatattacacatgggaggacccatacacattgttatttttaaatttaacattgaacaaaaaaaaggaggaatcacttaacttggaaacaaaacaccaaagttagtcagtggaatggtcaagagcaggacaagcctgccagggccactgaactccagtcacaagtggtcaaaagtatgaccgagaacaactggatgcaggaacatcagcacaaaatacagatgggaatattctcgggagtggtacatcaccatcttgtatgtgagggatattgtgtcagaggggaattacactttcaaaataaccagggctagtaggtcattagtaccagatggggaacagcaatggtgtggcatgccttgtttaaggcctgcgctggtttatctaaaagtagatttctgaaaagattgtccagtagcatcagaaagagccactttcagaggaaggactccttgttcacccacatcagactgcgtgtctcgttgggcttgcattcatactcaatgacagagaaggggcttccccactggcagtgatcccgcttgtggtaattgtagaacttgacttgccacactgtctcgaaggtcccaatgtctgtgaactgagggaagagaaacatgcctgataaaaaacctcattctgccacgagcaatagatgagagctaacattagtgatgtagctcctggataatcaagcagacccagtccagattctcttggtctgcaaggctcggaaggctttcctttagtggtctagctccctcctttatgatgggagatattttcctaaaaagctgtctgtaaaccattcttctctataaaatctgattcccattgcaaacgtgccactcgctatcatattgcttttaataaaaataactgcaaagtttatcttgtttttcaagtttaattttttgaaaacttagaattttaaagcttactctaaaatggcccctgctggccaggtgcagtggttcacccctgtaatcccaggctgaggcaggatgctagcttgaagccaggggttccagaatagcctgaacaacaaagcaagattctgtctctaaaacaaacccccatgttcatcccccaaatggtccctgctacagaaggggaatggcgaagggaagagaaggaataaagtttactggcttttctcaaaatacaatggtacaaaacaacctgtctacaatacatctacataaaatttccatcattagtttcaattccaaagaaaaatatttcaaagtgataactgaaaatatttcattggaagtgttaaaggccatattttaactattaaacattgaatacttataagtcaggagtccatgcctacatcttgggcttagattatacattacccagaaccatatttgagtatctgctatgtaccttgcatgtataaaaattcgtataaggcaggattgttgctcttaagaagttagcattctacttccagaaggtaggccctgtaaatcactgcacagaactctcctgaggtataaagtacacatttctcttgcagctgagaagagtacctggaataccctggaggccactgggcagatgctcaagtggcctgtggggtagcctgaccctgctactgccttctggagctatttaggggatctggcattaaactgcatgatgcctgtcgttattttctcctctgaccctcccctgtcatgggaggggacagggactgacttggcaggactcttagacttgtctggagccagaagacgtggacagaggaaggcagtgagcattaggagtcttatgcttcttgctgatacaggaggagggtgatcaggtgctttccagatagctttccaagatgctctcaaaattatatcttgcaccattgcctcatcctgctaatttgatcccaaaacaagatctaggctttcctctgggaaatttgcttgatttgaaatagtggatgatatacctagagtccacagacctcccaggtatgaaagtggattacctctttttagagagtcatggcttaaagacctatataagcatacagcagctttattcacaatagccccaggttggaattgacccaaatgtttattcacatgttgataagcaagttttggtatatttataaaatggccttttgctgaacaataaaggaagacatgctgattcaagcagcaacagggatggatctcaaagacatgctaagcaaaataatctaggcccccaaagtacatgctgtatgaatccatctatatagtgacagaatgtaacctacactgacaggaaacatctgtggttatctagagcctagggtgagaaggggaactgatgcaaatgtgcaagagggaactttttaggggtgatggaagcattctgtatcttgattgggatggtggttacatggctgtacatatatgttggtcaaaactaattgaactgtatgcttaagatgagtccatttattacatgtaaactatacctcaaagttaagaaaaatagaaatatccatttttagtgagctataagaaaacaggtattctaaatgctgctaatgggaattgaattagccttttggaaggatcatttgcatgaagttaaaggtgtgagtacattcaggcccagcaattccatttataatttatcttaagaacatcacaaagtttagagtagtaaacaatgcaacactaaaggtccatctttaaggcactatttgtggcatatctatataatggaataccatgtaatatttttaaatgtaaagtatttaatacagtttttattttatattgttacatgaactaagcagaataccatgaagaacatgaagtattaattcagttaaaattaaacttggctaggtgcagcagctcacacctgtaattccagcactttggaaggccaagatgagaggatcgcttgagaccaggagtttgagactagcctgggcaacatagtgagaccctgcctctacaaaaaatgtttgaaaattagctgggtgtggtggcacacactgtgagactgagataggaggattgcttgagcccaggagtttgaggctgcagtgagctataatcatgctactgcattccagcctgggcaataaagaccctgtctcaaaaaaaaaatgtatgtgtatatatgtgtatgtgtgtgtgtgtgtgtatttaatgttagaaatcctgatggactgttttattttctcctatgttctttttttaatttttattatatttttgagatggagtctagctctgtcgcccaaggttggagtgcagcagcacgatctcggctcactgcaacctctgctgcccgggttcaagcaattctcctgcctcagcctcctgagtagctgggattacaggtgtgtgccactacgcctggctaattttttttgtatttttagtagagacggggtttcaccatgttggccagtctggtcttgaactcctgacctcgtgatccacccacctcggcctcccaaagtgctggcgtgagccaccacacccggcccaacttctatattttttagcagagacgcggtttaaccatgttgcccaggctggtcttgaactcctgacctcaagtgatccaccagcctcggcctctcaaagcgctgggattacaggcatgaaccactgcacctagcagattttcttctatattcttatttgtattttcaaaatttaacgattactttaccagaaaaaaaattaaaccaaaactgtatttcccaaaagtattaccaaatagtcattatccttaatataaaggaccatatgcattattagtaagtactacgaggaacattggaataaaaaattggaatggttcattaaaaacaaaacaaaacaaagcaaaaaccataggctgggcgcagtggctcatgcctgtaatgccagcactttgggaggccaacatagtagtaccacttgaacccagaagtttaagaccagcttagacaacatggtgaaaccctgtcactacaaaaaattaaaaacattagctgggcatgggagtgtgtgtctgtagtcccagatactcaggaggctgaggtgaaaggatcgcttgagctcaggaggcagaggttgcagtgagctgagatcttgccactgcattttagcctgggcaacagggcaagacactgtctcaaaaaaaaaaaaaaaaaaaaaaaaaaaaaaaaggaaaaaatgactaacacctgcaaaaatattcaaatgtaattagtaattaaagaaatataaacttttaaagagatactattcctctaacaacttagtgacaatttctgagtgccagaaacaatctaagcatccaacattaggaaaatatgaaaatggtggcatatccatatgataaacttacagagcaattaggaatttagggttgtcgatgattcaaggaaatgcttagaacaatgtttaaaggaagatgaattggaatggtttatatacattatgagctcaaatatgtaaaataagatgtatagaaaaaatgaaaaatattaccttctaagtaaagaaattgaatttttctatccttactcagattttttctacaataaatgttacttttaaaatcagaaattttttaaatggtgaaatggatttatcatcttacaataatatatacagcctgcaagcctaagggattttataaattctattaaaaatatgtacattctaatgaaattatgactttgttcacaataggttagtggtggtatgtataaatcaaaatctcattttataatttaccaagacttttaaagaggatttagtattatttttgaccagcacaaggcattgcacactttaagtgcaactgaattttacaattcaattaacattttaattccaaattataaaaatattttgtaataaaatatacactgaaatataaactttatactaaaaatattttataatacttttcaaatgccagctaatattttgaaggaagcagcttaaaatcatgctaggtaacagagaaacaacactgagatttttattttttgaaataaaagttggttataaaaataaaaagcccaccaagctgattacaaccagacaagaaaaacgtaaggcttatgtctaaaaaaattttcaaaagtctcaatctaagaattctcagtaacaaaggagaccaaagttaaaatctgccaatagcaacagtcatagctaaagatatatttaagtgttcatattaatatttcaaatttggtttattaatgtgggacctaaggtcacgttgaagtaaatgtaactgctggtaactatgcatatgtcttcctctccccgatgccttagctgtttatacttctcattagccgtcttcctattctttcttttggagctctatttcctgggcctcttactacctagctcataatttcttcttgtctggctagaaacagatggggtcttgaaagctgacattcttcttaaagaacaggaacaattatacaggctcttcactccctccccgcccatctgtcccttctaaggagaatgtacccaggcaggctcctgggtggtacaatttatgcacaggtataattctgtatatttgtatagcaccctattttatgtgatactttcacatacatcattccactttagtcttgtgcctggttttattatctccagtttatggacaaaaaaactaagattcagagaggttagatgatttgccctcagtctagactccaaagccagccaatgataggagcaggacaagaacctaggtctcctaattcctagcttaggagctatttatcactgtacaaaactgctgatataaatggttatagtggacagtttttttttctccctgaatgagaaattacatgagttgtcacaggtagaatgctttagggcattgctatggactgaatgtttatgccctcctccaaccccaattcatgtggtgaagcctaaatcctaactgtaatggtatttggaggtagggcctttgggaagtatgagggtcatgagggtggagcccacatgaatgggttcagtgccctcataaggtacatgagaaagatgatctcttcttctgccgtatgaggatacatcaagaaagtgtctatggaccaaaaagagggtcctcaccagactactgccttgatgtttggtcttcccagcttccagaactctgagaaatagatgtttgttgtttcatccatccaatctatggcattttgttttagcagccccagcggagacaacactctacctggacattttatttgctgtaatgttcttacgagttgcaagcctggtccactttttacttcacaactaatgtggaagagaacacatctgattccaaggagaggagactcatctcttgtcctcaataacaaatgaggaatctgaagagcttgttttaaacaattttggtggtcttacaacccactggcatagaatctcgagtgtacttctttctattcaccatgccacctacttctactgcagacatgaaattaatcaagatcacgtagaaatccattggcttgggaggacagaacaatcttattcatagaaacatgatgttactttattagtaaacaaattcagtataaatatgcatctgtgacttttagtgtggtgtgacctatcaatttccactgaatttcaagcctttctattcctgaattgtttgaggttttattagtaaatgtttcccatttctcttcactaaataggactaaaagttctaggtcttataaagtacatttgtctttataagcaactgttaaacttttcctaagtgattgtgcccatgttatattctcaccagcaatgagaatttcaattgctctatatcctctccaacacttggcattgccagtcctttcaattttagccagtctggtgactatatagtgataagacaaagaaaggaaatcaagaggccgggtgcagtggctcatgctggcaatcccagcattttgggaggccaaggcaggtggatcacaaggtcaggagttcaagaccagcctggctgagattgtgaaaccccatctctagtaaaaatacaaaaattagccgggcgtggtggtgggtgcctgtaatcccagctactctggaggctgaggcaggagaattgcttgaacctggaaaacggaggttgcagtgagctgagatcacaccattgcactccagcctgggcatcagagcaagactctgcctcaaaaaaataaaaaaagaaaaaagaaaaaagaaacagcagaaatcaatgaattagaaaagaaagactatagagaaaacaatgaaactaaaatctagtttttttgaaaatatctttaaaattgataaacccctaacaaaactgagcaagaaaaaatacaaatattaggaatgaaaaagaggacatcaccatggatcctacagaccttaaattaggaaaaccttatactaataatttcaacaaaatggacaagttcttccttgaaagacacaaactatgagttgacatctacctaatgaggaaattgaatttgtagttaaaaatctctcaggcccaaatggctgcattgataaattcagacatataaggggaaaaagccaccaattccacataaattctttcagacagcagacagaacagttctcaaataattttatgtagccaatattgccctaattcctgacaaaaatattacaagagaaattacagacagattcctcatgggcatatacgtaaaatttgtttataatttttatttgcttgaagtattggtaaatcaaatctagcaatatatactgagaataatatatcaaaaccaacagggatttatcttctgaattcataagtggttcaacatttgaaaatctaccatattaacagaataaaggagaaaaaccatataattatatccatagttggagaaagagcacctgacagtgcatcattgattctattctcagcaaaggaaagtttttcaacttcattaaggggcattgacaaaaaacctatagctaactttatccttaatggcaaaagaccgagtggtttccctgtcagatgggaaacaaggcaagaatatgtgttcttactgcccctattcagcattgcattccaggtcttagccagtaccttaaggcaagaggaagaaaagggcggtggggagagtaaaacggtctatccagtgaacaactgtgtccgctaaaaatactaagcaattgcctggtgaggtggctcatgcctgtaatcccagcactttgggaggccgaggcgggtggatcaagaggtcaggagatcgagaccatcctggctaacacagtgaaaccccgtctctactaaaaatacaaaaaaattggcccagcgtggtggcaggcacctgtagtcccagctacttgggaggctgaggcaggagaatggcgtgaacccgggaggcggagcttgcagtgagccgagtttgcgccactgcactccagagcctgggcgacagagcgagtctccgtctcaaaaaaaaaaaaaaaaaaaaaaatactaagcaaccttaacaaatagccagtagaataagtgaatttagcaaggcttcaggatacaatattaacttataaaattcatatttcataaagaaatatgccagccctgaacaattatctttattatttcaccaattttacatgaaagttgacattgtattacttaccaatagtgagataaaaccatttttcatatatttattggccacttgtatttcttttataaattgcctatttatatcttttgcttaatttcctacatcttatgattatctcttacacatttctaataatttgttgtataaagtcacagctttcaccaaatactagaccagacagctgtcacgaggaccaacttctatatttggaaggctatttccctccactcacacatactcagcctcattaccacctcatatacctccctgccttcttcctccaaactctcaccagtctcccagtgaagagggccaacaaaagggattatgatgatgattcgtgcagacagactctctatctggcctttctccttggagggactgattgggaattagtcacactgctctgtgaaatggggtttgagaggcataaagttgggtgtcactttattagctcccagtaccagggcctctagaattatctgaagatcattgccattaatctaagtcagatggcatttgactatttcctgacttcagtggccactcacagcttcagcaggcagtaaagggctgtgaaatgtcattgacagtagctgcagtgtcactatgggcagtggcaaatcaaggcaacacagcttaggagactatacatgttatcaatgattttttcaccagttcactttgaagtgtgatatcagaaaagaacagtaacgtttcccccaaatagccagttatcccagaatcatttattcaatgatttgaaaaactttttttttttttttttttttgagacggagtctccctgtcgcccaggctggagtgcagtggtgcgatctcggctcactgcaggctccgccccccggggttcacgccattctcctgcctcagcctcccgagtagctgggactacaggcgcccgctacctcgcccggctaattttttgtatttttagtagagacggggtttcactgtgttagccaggatggtctcgatctcctgacttcgtgatccacccgccttggcccccaaagtgctgggattacaggcgtgagtcaccgcgcccggccgaaaaacattttaaactacttggtatcaactttggactcttaagcaagactcaaaagacaataatagctaggatatgtcatcagagttgaaaggaaatattatcttaaagcagaactgcaaatactaaaattgccagaattttaatgaggtcttagagggctaataagctacctttcacttaaaatatcctgagcactgggtaagtgtggagaaggtcatcataaatgagactgtttcctctcttccaagtactcagagggcagagaggagacctccccaactcctctctggagctgacctggcttggggagacctgagctgtaatttcctgttcacctcctcatgctactactgctccctctgggcctctggtcttagcaggttggattcacacttcttttgctttagatgtaaacagaacactaggaaagaagattaattcaaacagtttcaaaagcactcgattcaaaacttcttcctatttggatctgtaaagtaagaatttctatatctttttgaatagctgaagcacagggtcttcttctgttttaatgtgaaaatgattcagacttagtcattaaaacaaagatgttattacctaccataagtcttatttgcctgtccaggcatccattaaaaggttggagagtccaggcgcggttgctcacccctgtaatcccaggactttgggaagccaaggcaggcagaccacctgaggtcaggagtttgagaccagcttggcaaacatggtgaaaccctgtctctactaaaaatacaaaaaattagctgggtatggtggtgcatgcctgtaattccagctacttggaggctgaggcatgataattgcttgaacctgggaggcagaggctgcagtgagccaagatcacaccactgcactccagactgggcgacagagcaagactgtctaaaaacaaacaaaaaaaacaaacaaaaaagccagccaggtgcagggggtgcatgcctgtaatcccagcactttgggatgctgcagcaggcagataacctgagttcaggagttcaagaccagcctggccaacatggtgaaaaccctgtctctacttgaaaaaaaaaaaaaatacaaaaattagatgggtgtgttggtgggtgcctataatcccagctactcaggaggctgaggcagggagaattgcttgaacctaggaggtggagtttgcagtgagccaagatcacgccactgcactctggtctgggtgacagagtgagactccatctcaaaaaaaaaataaagttggagagccttagcttagttactagatgatttgaaggtatgttttaagaaaaaggtctgcggtccctaactgtaactcgtgaatccagcagtaaaggctaggtttctatatgtgaattttcacatctagaagcagcagctgattgacagttgattgttccccaccatgatcacataggttttggtttgagcactggtcatctggttgtcttttttttttttttgagacagagtctcgctctgtcacccaggccggagcagtggtgcgatctcagcttactgcaagctccgcctccaaggttcatgccattctcctgcctcaacctcccaagtagctgggactacaggcgtgtttgtatcttatttctaataggctgataataaagaaaatgattgaaaatataggttttctgatcaagtcaccagctcctatattaagttcaaatttttattctagtcttgaagctttcagatagccaacacaatccaatgacattttaaagtcaccaacatttcaaacttatgtattaaaaaatatagacactgggccgggtgcagcagctcacgcctataatcccaccactttgcgaagccgaggggtgtggatcacctgaggttcggagtttgagaccagcctgacaaacacggagaaaccctgtttctattaaaaatacaaaattagccgggcatggtggcacatgcctgcaatcccagctacttgggaggctgaggcaggagaattgcttgaacccgggaggcagaggttgcggtgagccgagattgcgccatcgtactccagcctgggcaacaagagtgaaactccgtctcaaaaaaaaaaaaaaaatatatatatatatatgtgtgtgtgtgtgtgtgtgtgtgtgtgtgtgtgtgtgtacatataaaacatatataatatatatttaaatatatatagacaccacactctcatggacaattgtgaaaacttgacatgtccaagtaaatccaatcaattaattagcaagtccctggattattcagatttcgtcaagggaaatgtcatcagtgtgggtgcctaagtcaagtcagaaatgtcagaattaaaactggaagtaggccgggggcggtggctcacgcctgtaatcccagcactttgggaggccaaggcgggcagttcacgaggtcaggagattgagaccatcctggctaacacggtgaaaccccgtctctactaaaaatacaaaagaattagctgggcatggtggcgagcacctgtagtcccatctactcgggaggctgaggcaggagaatggcatgaacccgggaggcggagcttgcagtaagccgagatggcaccactgcactgcagcctgggcgtcagagaaagactcttccaaaaaaaaaaaaaaaacacacacacacacacctggcagtaaagaagatattaaggctgggcacggtagctcatgcctgtcatcccagcactttgggaggccgagacaggagaaccacttgaggccaggagttcaagaccagactgagcaacatagcaagaccttgtctctactaaatataaaaaggttttgctgggtatggtggtgcgtgcctctggtcccagctactagagaggctgaggtaggaggatcagttgccagaggtcaaggctgcagtgagctagaatcatgccacttcattgcagcctgggcagcagagaccccacctctacaaaaaaaaaaaaaaaaaaaaaaaaaaattagctgggtgtgggtggtacgtagtggtagtcctagctactcagcaggctgaggtgggagaaccgcttgagtctgggaggtcaaggctgcagtgagctatgattgtgctgctgtactccagcctgggtaacagtgagataacagcctggtaatagtgagatcctatctcaaaaaaaaaaaaaaaaggaaaaaaaagagtaccaggatactgtttacctctaattaactgtgtgatcctagacatatctgaactcgtcaggcctgagttttcaatgaaaagtgtattccactatctaatctctaaggccacttttagctctatcatggtataaagattacaaatggcgaccaggcacagtggctcacgccagtaatcccagcgttttgggaggccaaggtgggaggatcacctgaggtcaggagtttgagaccagcctgtccaatgtggtgaaaccctgtctctactgaaaatacaaaacttagctgggcgtggtggcgggtgcctgtaatcccagctactcgggaggctgaggcaggagaatcgcttgaacctgggaagtggaggttgcagtgagccgagattgcaccattgtaccacagcatgggtgacagagcaagactccattaaaaaaaaaaaaaaaaattacaaacttacccttaagggtattgcattatgtattttttgaagaaagtcagattctcgaatactgtcatttacaatcacaccaaacaggacagaaggccattcaagtacctgtagaattcctttttggttgggcacagaggggaaggctaggaatgtgtcccagcatcttagatgatttgcatgattttaaaaagtgaaataagattcataaatttgagcaaatttttaaagtggcagtaatccttacattttgattcagttgattttcagatccccttcacatatacgtattttacagttgcaattatttaacattaaatgttatacaattgtatatattgtctaactacatatcattgttacaattttgtcaaaatgttatacaattgtatatattgtcaaactattgttacaattttgtcaaaatgttatacaattgtatgtattgtctaactacatattctttgctacaattttgtcaaaatgtataggattgtatatattgtctaactacatatcgttacaattttgtccttgtttgttgaggcaggatcactctgtggcccaggctggagtatagtggtttgatcatagctcactgcagccttgaatgcctgggctggagtaaccctcccacctcagtctctggagtagctgagactacaggcatatgtcaccacgcctggctcattttttattttttattttgtagaaataggattttgctatattgctcaggctagtcttgaactcctgcccacatgcgatcctcttgccttgtcctcccaaagtggtggatttataggtgtgagccactgtgcctggcctcattgttccaattttaatgatgacataggactcttatcgtgatgtatcatgacaattagactactccccagatgttagatatgtcagttgccctctaacaaaaagtgctttaataaataattttgtatatatgtctactttaataggagtggaattatttggtcaaaagaaatgaatctctatggctgttcccacatatgaccattttgctccttttaaagtatacaaacaatcttacaatgttaacagcaaagaaggcattgtttagaaaaatgatatcattgggccaggtgtggtggctcacccctgtaatcccagtactttgggaggctgaggtgagaggattgcttgagcgcaggagtttgagatgagcctgggcaacatagtgagactccatctccacaaaaaatttaaaaattggcttggcgtggtggcatgtggctgtagtcccagctattcaggagtctgaggcaggaaggtctcttgagcccaggagtttgaggttacaatgagctatgatcatgccactgcactctagcctgggttacagagtgagattctgtctcttattaaaaaaacaaaacaaaacaaaacaaaacaaaaaaaacccccgatgtcattgggaattacttttaaaaaatagatttcctagtttacttttcactaccatttacaagaatgcacatttctacattttaacttagtttcttcttctggaaaatgttttgtcaatatttctgttgaagattggtatttttaaaatatcacagactcttgaagaagtgtcatatttctattgttttattctcactttatactgtgtagagacataatttcttatttttctttatgatttctatttaaaaatcaagttaacttttctagtctattttgatttttccgtaattttagattttataatttaactttcccaccttatttaaccttaatttatatatttatacctatatcattttacaaagaatttaagaaggtatttagcattttatataaaatgaaaaaacacacataatgatggtctgaaatataagcatattaaacataatgcttggtttgagcttagatttgatgctgggctgtctggttgctggtgcaaaaattaaaaaaacacagattgtcaattatcagtgattttaatgtttttgcagaactttttttttctacccaataagacagcacatgactagatgaacaagagagatgcctgcctctccccacccctttcagtgagcactgaaatatctcaggtggatccagaattcagaaaacagtccgaaaaaggagttaaatcatctcatcattagaagggagagaaatattctcagtttcccagaagaaaaaaaagttaaaaagcctttctagctctaatttgtaacaaaacaaaacaaaaataaaaatgaaaacactccacatgaaacatttagggcatttagctgtgagctgtttttacacacagctatttcttagagcaaactttcataacctttgaaagcccagtgtcaatgtgcaactcagttaaagtgcttctaaacttgtttaagggtgttgtttttgatggcttaactggtctatcagtagcatttaatatttctagaggtctaaaaatacattttaaaattctgtagaaatggatgttttcctcagaaaaacataatagtcactcaataaaacccccaaaaggcaaaagtcatgtgctaaattgagaattcaagacattccttctaaatatctttatttttggtcctgacttttatgggaaatttctgtcagactttcaagtaacagttaagttctatggtatgtaaattgtttcagagcataaactataaaaaagttcctaattcacttcacaaagctattaaccctaataccaaaatttaagactgcaaaggccttctaattgttctttgcttttctgtctccacttctaatctaagcactggcactagagtgcttttaaggtgcctatcttatcacattaccagaaaggggtacttctcaaccccttgaaagactggcaggagaaaatgacagccctatctgttggaagtggcagaagacagcatcaggactggcagagaagctggaaataacctagaaaaatcctggaagaaagagatctgcagaaggaatagaaccccaaatctgactataaactctgctgtaatccctagctaactgcatatgaaggatagtgggacatggacaccctacaggggccattgaaaaagcagacaaaccagcaaaaagtcaatccttgaaagataaagttgtgctaagattaatagcttctttatatttacgtatgtatgtatgtatgtatgtatgtatgcatgcatgtattttgagtacattccctaacctcagaagtaggtatcagagtaaagagttttggattggccaggcacggtggctcactcctgtaatcccagcactttgggaggccgaggcaggtggatcacttgaggtcaggagttcgagaccagcgtggccaacatggtgaaaccctgtctctaataaaaatacaaaaattagccaggtgtggtggcacgcgcctgtagccccagctactctggcggctgaggcaggagaatcgcttgaacctgggaggtggaggctgcagtgagctgagatcgcaccactgcattccagcctgggcaacaaaagtgaaactccatctcaaataaagcaaaacagttttggattgatagagcagctggagaaataggggaacctcagggtgggtatatccaaaaatctcagtataagctctccagttccttggctgactgctaagctccatgggcacaaaaggagaccaggctagaaaagcagcaactggaagttataaaaactgagcaaagagatcagctgctgcatactgctggaagatagaatttgaagtttgagtacaggaatgtttactgccagctagaatacaaaaaaccagtaatacccagaagaataaagcagaaactagagttggtaataaactatccacaatgttaagtttgtggccaaaaattacgagacatgtaatgaaacatgaaagtgtgacccatattaaaaaaaaaaaaagtcaggctgtaataatttactctaactagagccaggtgttggatttattaactacttcaaaccagcaattacaaatgtgctcagataaattaaggaaaacatggtctcaattagccaacataaggaatcttgagaaactgaaactataaaaaagacaaatgaaaattctagagctaaaaaatgttatagaatttagaagataagcacaagactagaaatagaagaatcagtgaacttaaggatacatcagtagaaatgatctaatctgaagatccattaaaaaaagaaagaaaattgaacatgtcctcaaagacttatgggataataccaagcttttcaacatatgtttagttggggtcctacaaagaaaggagagaatgaagtattaaaaaagtatttaacaaaacaatggttaataatactccaattttggtgaaaacattaacctctagattcaagaaattcagtgatccccaaggggggaaaaaccagttaagatgcatcataaccaaactactgacaagcagaaagaactctatgaagggatcattttactgtatatgtattatacttaagttaaaatatttcaaagcaaacaagggaagggaataaatataggattagaaaacaatgaagtagaaaaacagagtgaagcatgtgaaaaaaaaagctaggattttgaaaaggccaataaaactgaaatctctagctagactaatcagagggtataaaaataaaaattttgaataacaagagtgaaagagtaggtcgggcgcggcagctcatacctgtaatctgagccctttgagaggacaaggtgggcggatcttgtgaggccaggagttcgagaccagcctggccaacatggcaaaagcccatctctactaaaaatacaaaaaattagctgggtatggtggtgcgcacctgtaatcccagctacttgggaggccgaggcaggagaattgcctgaatctgggaggcagaagtttcagtaaaccgagattgcaccactgcactccagcctgagagacagagtgagactccatctccggggaaaaaaaaaaaaaaaaaaaaaaagaaaaggatcaaagagtaaaagagtagacattattagagatcctaagacactgaagaatggcggagattataaacaactttatgggaataaagttgaaaatttaattgaaataaatgacttctaagataaattacaaaaactcaggaaataaaacctgaatagcccgatatctattaacgaaattgaatttgtaataaaaaatcttcaaataaagtataatctatgccttcattggtgaattctgacattaagaaaaatatcatttctatataaactcttccagaaaatagaagaatgaacactttccgaatcattttatgtagccagcattactctgatacccaaaactagacaaaggcattataagaaaagcaaaccatagacaaatatcccccccggaacatagatgtcaaaaatccttaacaaaatattaatgaatcaaatccagttttacataccgagaataaaccatgaccagtggtatttaacctgggaattcaaggttattttaactttgaaaaagcaaggtaatctaccataaacagaataaaacaccttatgatcatttcaaaagatacagaaaaactatttgaaaacatgttataatacgttcatgattaaaaaaacattcagtaaaccaggaatagaagagaactcctttaacctgataaaaagatttacaataaagaaccttacagctactatactttgtattgaaaaacagaatgctttctctctcagactgagggacaagaatgtccactctcaccatttctatttagcagcatactgcgaaggtcctagccattgcaattaggccataaataaaatgaatacagattggaaagaaaagagaaactatttacagatgacatgatagcatatttagaaaatcctagggatcctccaaaaacagctattaggattagtgagattagctaagtctcaggattcaatatcaatatacaaaaaaaatcgattttatttccatataccagcaaagattccatttacattattcaaaaatatgaaataataaatttaacaaaatatgtataaagtctgtagaggaaatactgtaatatattgtggagaaaattaaagatgacctaagagatatttcatgttcatggtatagaaaactctatattaagatgtcaaatatctttaagttgacctataggtttaatggaaactctatatcccaatttttggaggaattgacaagctcattctaaaatctatataaaacacaaaggacctagattaagcaaaagaacttagaataaacaaagctagaagatttatgctacctgttttaaggactacaatatagatcaaacaagattgttttgggtcttctatgtctttggaatttccaaattaaaaagacatacattgagtatgtaaagcaactagaattctcatacactgctggtggatatacaaaatggtacaacagtgtggatatttcttacacagataaacacatacttaccatgtgatttagcaattgtcctcttaggtatttactcaagagaaatgaaaatatgttcacactgagacttatactcaaatgttcacagcagctttattcataatagcaaaagctgaaaacaacccagatgtccatttattggtaaagggagaaattatggtatatccacacaacataatatgctacccagaaataaaatgaaccaactactgatatgtgcaacataggtgaatctcaacattatactaagtgaaagaagtcaaacagcactgttccatttacatgaaattcttacatagttaaaactaacatattgagatggcaagctcaggataggttaggttgggaggaattaactgcaaataattttcagggtaatagaagtgttctgttatctttattggggtggtgattatatagtagcatctacttgtcaaaacctatgtaactgtaaagttggtgcattttatgttaatcatatctcaaagctgatttcaaaaacttcaatgatttcctatagtcattaggataaatccagtggcaaaaaggttctatatcatccaacccctacttacctctctggctttatcttatactgtctttgcctcactctatgcccaccatgttgggcttctcatccattagaatacatacgctgtctccagctcttgacttttacaactactctttcctcaaatgctctttctttcctcaactcttattcatccttcattttttcctcaagaaaaccatcccgcaaatcttggactaggataagacccttggtatatactctaacagcactacttttttgctatacttttcatattagcaattatttaccttatcattgataatcatgtataaagtatgtttccaccctcaagatcagaaattctatgaaacccgggttatctattttgttcaccactcttccccctgagtccctgatatacaggaaatcctcaaatatttgctgataaagcaagcaaattttaaaaatctaaaaattctaaataaaagagcatatagaatttagcaatatattaataatacatgtttaagtagggcttattgtaaaaaataaggtttagtatttggaagtgtattactttatgtaattcatcaaatatatcaaataagataaattatctccaactactgctactagctgtagttgactgctagcagcagtcaacattattctagaaacattagacaatgcaatatgaccaggaaaaaaaaagacagttatattcagattctgaccatctatatgaaaaacccaagatgatttgctgaaaaattttagtaagcaagttcagtaaggtggtcagttacaaagtatatgaatacatattataaaaacagtgtatctaaaacaatgcagtattggtgtaacaatagatagatcaatggaatagattagggagtataggaggagataaaatttttgcaatataagaaatttctatgaaactgacctttggagaagcatagattatttaaataattgtattaggacactgtgggcaaatactttccaatataaaagtaagaactgaaataatatctaaatctgtaattcatctaaaatttaacaaatgagaccataagagttcagatgaaaatacacaaatgccactgtgtatatgtctatatataattaattaatctgggagtcaaggaagactttctaagctggatcttaactgcaaaataataaaggaaaaggctaacatgtttgactacaaaatttatattttatattgtaaacaaaataaaaattaaaatattaaatgaacactcaagatgtgataaaaatgacgattttaaatttataaagatttttcaaaaaatcaataagatacacttaagcagaaaaatgggtgaaaactatgaacatataatccatgaagcaaatacaaatagtaaatgaatatgtcaggatttcaaattaacagtaagtaaaaattgaagtatttcagtcagattaaaaatatgtaaacatgacaaactatacctagtgcagagagaaatggatacttaaatacagatgataggcgtgtaaattgcctagcttttctggaggtcagtgaggcagtcttataaaatgccttaacgtgcacatttcttctagaaatcaagaaaacattagacaattgtacagtactcagtgatttgtaatgtggttttgtttataataccgaaaaccagaaataatcagcaataagtgactgaggataatttgatacattcataaaatacaatacccatacagactttaaaatgacattccatatatatatatgggcatatatttgacattgaaagatgtatacaagtagaaaaaagtatatatatggaatgatactcaaatatatatatttaatgacagggtcttactcttgtcaccgaggctggagtgcagtggtgtgatcatagctcactgcagcctcaacctcctggatccaagtgatcctcctgcctcagcctcccaagttgctggcactacaggtgtggtatcaccacacccggttaactaaaaaaaatttttttagagacagggtcccactatgttgcccaggctcacacatatatattcaaagacatagaaacaactttctaagaacatttattgacatgttaacaacacttctctcttgcaggaattatacatttccctcatttttgtatttttctgtgttgaaggttctttgacagaacaagagcactttctaaaaatagttcaaacattggatgcaaagggatgagctgctagattatttccttatgtgactattgtggttttaaaaagtataatggggactgggaacactttaagtttccaaaggttagataggcatttctcaaagtgcattcccacaccatcctcttcaggatctggtatgcttgctaagagtttctctgtccccaccctagctctactgagctaagatcactggagagggaacctaggactggaattttaaacaaactcccaaagagattctcacgcataccgatatttgagaacccttgtgatacttcctataaagctagagggcagacagttgaaggttgttcttgcagagggaaaaggctgcccagtttgcaagcagcagtttaggtgaagcttccttttcctgtgctttagcaagcttgcttcatcacctatttattatgtttgcacatatagttaaatctatgtcttgactctatcctttctaccaatttgttttttcttacttggggccatattaccagggtatatcattttattatatatgatatctgataggattaccagatattatatataataggtatatataatataggtatatatatattatataggtgtatatataataggtatatattatataaaataggtgtattataggatattatataagaggtgtatttttcccatgacactttttttggctcctccatttagctttaagtagcaaagtgtcattcaaatcagctttctcttaaccagaatattgtggcctacttggaatcagaaacttttcacagccaattatctaaaccagttctgaagtagactataactgggaaaatggcaatgtaggttctgtctcttccagaaccatctgcttctgcttatggtgtcttttcagcatctacagggtcagtctgtcatgcaaaagagcattaattaagtagttctctctccagcagatgcttgaaggaaatccacaactcaatttccacagtaattggctcccaacacaccttttcttacagagagatagtgattgttttcttttctgtggtttccattttccatggcaccatactcctatacccgcaagcaagccttaccggcatggtgatctggatgggttgccgttctccactcttcgttggggtcacaccttctgtgtcatatgtacccgtataaacaactctgtccccatcaggctctttagacttcagttccagtgggacaagcacaccaccaatggaaatattcctgcaatagacttgagttgaccacatacggttgaaattagcctaggcaccctgtcctgttttggaccaaaactctaggcaggaaacttacagggtttcactaaggggggcattgtggtgcgggagaaagttctgcaagttcagggggcagtgcttctcctatttatgaattaaaaacctacctatccccagccccagacattaggattcagttggcctgggatgtagtttggacactggtacatttattttaaggttatctaggtgatcttaatatgcaggtaggattgagaatactgaactcgtcctctaatattttttttctcctgttccaacaatcaggggtcctatgatttggagtacctcaagccttctacaccatcagaaattctcactggcatcattcttttgtgttaggataatggttctcaaagccaagtccctggaccagcagtatcagcatcacctgcagaccttagaaaacaaattcttaggccctaccatagactaaactagaacttctagggagagctccagcatctgtgttttaacaagcactccaggtgattctgatatatactcaggtttgagaaccacaggattagagttgagaaatccttgatagcattaaaatgtaaccagaaaaaatagttttattcctaacaaattaacttttagattagtttgaacactagaggaatactctaaaaaaaaaaaaaagaaatatattaacagataactccggggataaggttggcaggaaagaagggaaaggaaaagcctggattatatccaggtgcacagaacactgggaaatgtgagaataaagggtgcatggagctgtgggggatgctgacaaatagatgaatcaggagtaccagaaaccatccagatttggttctaggtccttgcaccttggaaggctaatacaagtttactgaattaaaacgtaagctataagtagtatctgatgaattcaaagtccttccacccccgcaataatgcccaccatatgtagtagatacaataaatgctggtttattttctctctgcagactagactttggtaggtagaaacgagaggtaaaggggctccacccgcagttcttccgggtaggctactgcatgctcccacacactttcccgccagaggtctactgggtgcattatccagcggtcgctggcaagaaatcgtccctgccatggataaaccaaaaaacatcctcttgctgtggggatgttgctaatcccaggcaccatcaccagcctgaacagaaaactcaggagaaaaagaacagcatacggcatggaaaaccgtgaaactaggccagagaaggggcggccgctctcttactagtgtctgctgctccaccccagggtcccagccactgaatggcgaagggagtggggagcatccctcagggagccccagtaatcacccctcccctgcctttccacctcattcctcctttctccctccttcagccttgcgggcagaccctgtgggccgcctggaccgcgcgcaggagggctgggattgcggtggctgaaccctgcggacctctcccatctgctccaccccgaccgcctgcggttccgcgcccaaggctggacagaaggcaggagaaatttataagaaacagacaagcaaaaaccctggcttcttgtcactgattttaaagaacccactgaggtcactgcgatgggtggagggaagcgagaatggaggaatacaagccaaagggaaggaaggggacgaaggcggacagggagtgacctcttcctccaacccccgggcccgctgggagcggcgcgaggccagaggcccttgagaggctcgggctgtcctgggggcctcagtcctctgcctgtaccccatgggggaccctgctgccaccaggcgccccgcactcactcgacctgcagcgtgctgggtttaatcttcacctcaaccttgtaggaggagccggtgagcagcttgatggtgcggttctggccgaagcgctgcccgtccaccttgtaaaagaccgggccgtcattaggctggatgcgcagcgcgatggagaggcgcacgaggcccggcaggtcccccatgtctgggcgagggtctggcgcggcggctccggggggcggaggacagcgccggctgcggccgagtggctggagcgcgaggggcggagaggaagcgcggggagggtgagggaggtggtggagctgaggctgccgctaggaacccgcgccgtcgccgccgtccgcccgggcttttgaggagcagctccttaggctgtggcccccctccccactcggcgaggaagcgggcccaagagacggctccaaggccgcgcgcttccccatcccccgctccagtgctgcgccctccacgcacccgaaggctcgctctggcccgcaggccgccgcgcagatccgcgcagctgggggcgagggagttaatcctgtttacgcaccacaatccccttcagctggggaagcggacatttaggctcctcctagaacagccccgggcaggaggaggagaggtttgggaggcactgggaaggcgctggagttaagcgaccactatgccaaggagcgagacccccggaatctggataccgcctcggccagctacgtgaggtggacactgctgctcgcggatccggcgccagcca

SEQ ID NO: 4 - DNA, 495 bp, Homo sapiens
atgggggacctgccgggcctcgtgcgcctctccatcgcgctgcgcatccagcctaatgacggcccggtcttttacaaggtggacgggcagcgcttcggccagaaccgcaccatcaagctgctcaccggctcctcctacaaggttgaggtgaagattaaacccagcacgctgcaggtcgagaatatttccattggtggtgtgcttgtcccactggaactgaagtctaaagagcctgatggggacagagttgtttatacgggtacatatgacacagaaggtgtgaccccaacgaagagtggagaacggcaacccatccagatcaccatgccgttcacagacattgggaccttcgagacagtgtggcaagtcaagttctacaattaccacaagcgggatcactgccagtggggaagccccttctctgtcattgagtatgaatgcaagcccaacgagacacgcagtctgatgtgggtgaacaaggagtccttcctctga

SEQ ID NO: 5 - protein, 164 aa, Homo sapiens
MGDLPGLVRLSIALRIQPNDGPVFYKVDGQRFGQNRTIKLLTGSSYKVEVKIKPSTLQVENISIGGVLVPLELKSKEPDGDRVVYTGTYDTEGVTPTKSGERQPIQITMPFTDIGTFETVWQVKFYNYHKRDHCQWGSPFSVIEYECKPNETRSLMWVNKESFL

SEQ ID NO: 6 - DNA, 1295 bp, Rattus sp.
  modified_base (1077): a, c, g, t, unknown, or other
atgggggatctaccggggatcgtgcgcctttccatcgcgctgcgcatccaacccaatgatggccctgttttcttcaaagtggacgggcagcgctttgggcagaaccgtacaatcaagctgctcaccggttcctcctacaaggttgaggtgaagattaagcccagcacgctgcaggtcgagaatatttccattggtggtgtgcttgtcccactggaactgaagtctaaagagcctgatggggacagagttgtctacaccggcatatacgatacagaaggggtagccccaaccaaaagtggagagcgacaacccatccagatcaccatgccgttcactgacattgggaccttcgagacggtgtggcaagtcaagttctacaattaccacaagcgagaccactgccagtggggaagcccgttctccgtcattgagtatgaatgcaagcccaatgggacgcgcagcctcatgtgggtgaataaggagtccttcctctgaaaatggctcctcctgacagttgctagacaatctccggaaatcgatgcttagacaaaccagcacaagccttaaccaaggcacaccataccatggctgtttcccaacgacctcctatccccaattactgtgaaagtgtgattccctcctgaggcagtacctgtgacatcgcgtgggatgtcacgtccctgaaagttctccttccactattgtatactggtgttcccgcatctctcttgtctgccagtgtgaatggagctcagtgatagtatcaggtttgtcctataccagaaagttccacagaaggaccatagtgttttgtttccgggacaagaaatttctcccttttgtgttcattgtcaaattaagataacagtgtgctggagtcccccatgtgtgatttgtaacatgtagcatgcaatgtttgtgagcaccccaagaagaggtaataacataaacagcggttaacgggacccaaatgttgtgcttcccatcagcagctgtcagcttcttcagaggaagaaagatgatgaagaaataaaagagatgagtatataaataagagtttataatgtatcaatggtnttccaggnagcggggtattttgctgtgatntagtagaaacaggggaaagacactgttgtagaatcggagctgtggctcgcccctttgacaaactcccatagactttaaaccaacatagagatggcgcccctgcacaccctttcacttgaactttgtacaactgtgtatggaatgggtaatcaacttttgatatttcttaataaagacattgaaaaaaa

SEQ ID NO: 7 - protein, 164 aa, Rattus sp.
MGDLPGIVRLSIALRIQPNDGPVFFKVDGQRFGQNRTIKLLTGSSYKVEVKIKPTTLQVENISIGGVLVPLELKCKEPDGERVVYTGIYDTEGVAPTKSGERQPIQITMPFTDIGTFETVWQVKFYNYHKRDHCQWGSPFSVIEYECKPNETRSLMWVNKESFL

SEQ ID NO: 8 - protein, 164 aa, Macaca sp.
MGDLPGLVRLSIALRIQPNDGPVFYKVDGQRFGQNRTIKLLTGSSYKVEVKIKPSTLQVENISIGGVLVPLELKSKEPDGDRVVYTGTYDTEGVTPTKSGERQPIQITMPFADIGTFETVWQVKFYNYHKRDHCQWGSPFSVIEYECKPNETRSLMWVNKESFL

SEQ ID NO: 9 - protein, 163 aa, Homo sapiens
GDLPGLVRLSTALRTQPNDGPVFYKVDGQRFGQNRTIKLLTGSTYKVEVKIKPSTLQVENISIGGVLVPLELKSKEPDGDRVVYTGTYDTEGVTPTKSGERQPIQITMPFTDIGTFETVWQVKFYNYHKRDHCQWGSPFSVIEYECKPNETRSLMWVNKESFL

SEQ ID NO: 10 - protein, 184 aa, Mus musculus
MGDLPGLVRLSIALRIQPNDGPVFFKVDGQRFGQNRTIKLLTGSTYKVEVKIKPTTLQVENISIGGVLVPLELKGKEPDGERVVYTGIYDTEGVAPTKSGERQPIQITMPVSPSCGCGDMASEQRNCRGRNHYFFTNKERCAGASFYGNRVQFPSWTCEKRERVIFTDAFFFHERLTFKKIKIK

SEQ ID NO: 11 - protein, 60 aa, Mus musculus
MGDLPGLVRLSIALRIQPNDGPVFFKVDGQRFGQNRTIKLLTGSSYKVEVKIKPTTLQVE

SEQ ID NO: 12 - protein, 117 aa, Artificial Sequence
  Description of Artificial Sequence Synthetic consensus sequence
MGDLPGLVRLSIALRLQPNDGPVFYKVDGQRFGQNRTIKLLTGSSYKVEVKIKPSTLQVENISIGGVLVPLELKSKEPDGDRVVYTGTYDTEGVTPTKSGERQPIQITMPGYCSERF

SEQ ID NO: 13 - protein, 164 aa, Rattus sp.
MGDLPGIVRLSIALRIQPNDGPVFFKVDGQRFGQNRTIKLLTGSSYKVEVKIKPSTLQVENISIGGVLVPLELKSKEPDGDRVVYTGIYDTEGVAPTKSGERQPIQITMPFTDIGTFETVWQVKFYNYHKRDHCQWGSPFSVIEYECKPNGTRSLMWVNKESFL

SEQ ID NO: 14 - protein, 164 aa, Homo sapiens; identical to SEQ ID NO: 5

SEQ ID NO: 15 - protein, 130 aa, Homo sapiens
MGDLPGLVRLSTALRTQPNDGPVFYKVDGQRFGQNRTIKLLTGSSYKVEVKIKPSTLQVENISIGGVLVPLELKSKEPDGDRVVYTGTYDTEGVTPTKSGERQPIQITMETPECLEQRPQEISLTYECEE

SEQ ID NO: 16 - protein, 164 aa, Homo sapiens
MGDLPGLVRLSIALRIQPNDGPVFYKVDGQRFGQNRTIKLLTGSSYKVEVKIKPTTLQVENISIGGVLVPLELKCKEPDGERVVYTGTYDTEGVTPTKSGERQPIQITMPFTDIGTFETVWQVKFYNYHKRDHCQWGSPFSVIEYECKPNETRSLMWVNKESFL